(19) United States
(12) Patent Application Publication
(10) Pub. No.: US 2025/0325548 A1
(43) Pub. Date: Oct. 23, 2025

(54) TUMOR SUPPRESSOR P73 TRANSCRIPTIONALLY REGULATES C-FLIP TO IMPEDE ITS PRIMING OF EXTRINSIC APOPTOSIS WHILE AN EXAMPLE SWITCHER COMPOUND CB-7587351 DEGRADES C-FLIP PROTEIN

(71) Applicant: Brown University, Providence, RI (US)

(72) Inventors: Wafik S. EL-DEIRY, Barrington, RI (US); Shengliang ZHANG, Barrington, RI (US); Lanlan ZHOU, Barrington, RI (US)

(21) Appl. No.: 19/184,962

(22) Filed: Apr. 21, 2025

Related U.S. Application Data
(60) Provisional application No. 63/637,860, filed on Apr. 23, 2024.

(51) Int. Cl. A61K 31/517

(52) U.S. Cl. CPC A61K 31/517

(57) ABSTRACT

The present invention relates to methods for treating cancer in a subject by administering a therapeutically effective amount of a p73 protein activator, specifically a switcher compound. The method involves obtaining a subject with cancer, suspected of having cancer, or susceptible to cancer, and administering the switcher compound to reduce c-FLIP-L/S gene expression. This reduction sensitizes cancer cells to apoptosis induced by p73, thereby treating or preventing cancer in the subject. The administration of the switcher compound is configured to enhance the therapeutic efficacy by targeting the gene expression pathway, offering a novel approach to cancer treatment through the modulation of p73 protein activity.

### CROSS REFERENCE TO RELATED APPLICATIONS

This application claims the benefit of priority to United States Provisional Patent Application No.: 63/637,860, filed 23 Apr. 2024, the entire disclosure of which is incorporated by reference as if fully set forth herein in its entirety.

### STATEMENT REGARDING FEDERALLY SPONSORED RESEARCH OR DEVELOPMENT

This invention was made with government support under Grant Number R01 CA 176289 awarded by the National Institutes of Health. The government has certain rights in the invention.

### FIELD OF THE INVENTION

The present disclosure relates to methods for treating cancer, and more particularly, to a method involving the administration of a p73 protein modulator to sensitize cancer cells to apoptosis.

### BRIEF DESCRIPTION OF THE SEQUENCE LISTING

This submission will be accompanied by a “Sequence Listing XML” file containing SEQ ID NOs: 1-Y, which will be created in 2025, X KB (kilobytes) size, and will be submitted with the filename: “405505-776001US.xml” or other appropriate name to be determined. None of the sequences therein will contain less than 10 amino acids in length or less than 10 nucleotides (else will be flagged) and none are thus mandatorily marked as intentionally skipped sequences under WIPO Sequence software version 2.3.0. The Sequence Listing XML will be generated using WIPO Sequence software version 2.3.0 (or latest version), in accordance with 37 CFR §§ 1.831 through 1.835, and is herewith submitted as an XML file, via the USPTO patent electronic filing system, 37 CFR § 1.835 (a) (1). The Sequence Listing XML file will be incorporated by reference herein in its entirety.

### BACKGROUND OF THE INVENTION

Traditional cancer treatments have, in the past, primarily focused on methods such as surgery, chemotherapy, and radiation therapy. These approaches aim to remove or destroy cancer cells but often come with significant side effects and varying degrees of efficacy. Chemotherapy, for instance, targets rapidly dividing cells, which can lead to damage in healthy tissues, hair loss and can result in adverse effects such as immunosuppression and organ toxicity. Radiation therapy, while effective in targeting localized tumors, can also harm the surrounding healthy tissues and is not suitable for treating metastatic cancer.

Meanwhile tragically, cancer remains one of the leading causes of mortality worldwide, with millions of new cases diagnosed each year. Despite the significant advancements in medical research and treatment options, cancer continues to pose a formidable challenge due to its complex nature and ability to develop resistance to conventional therapies. Traditional treatments such as chemotherapy, radiation, and surgery, while effective in certain cases, often come with these severe side effects and may not be suitable for all patients. As a result, there is a pressing need for more targeted and less invasive treatment options that can effectively combat cancer cells while minimizing harm to healthy tissues.

The quest to cure cancer has been fraught with numerous challenges and setbacks throughout history. Early attempts to treat cancer date back to ancient civilizations, where rudimentary surgical techniques were employed to remove tumors. However, these methods were often ineffective and carried significant risks of infection and mortality. As medical knowledge advanced, the 19th and early 20th centuries saw the development of radiation therapy, which offered a new avenue for cancer treatment. Despite initial optimism, radiation therapy was found to have limitations, including damage to surrounding healthy tissues and the potential for secondary cancers.

The mid-20th century marked the advent of chemotherapy, a groundbreaking approach that utilized chemical agents to target rapidly dividing cancer cells. While chemotherapy represented a significant leap forward, it was not without its drawbacks. The non-specific nature of chemotherapeutic agents meant that they also affected healthy cells, leading to a host of debilitating side effects such as nausea, hair loss, and immunosuppression. Moreover, cancer cells often developed resistance to these drugs, necessitating the development of new and more potent chemotherapeutic agents.

In the latter half of the 20th century, the discovery of the genetic basis of cancer ushered in a new era of targeted therapies. Researchers identified specific genetic mutations and pathways that drove cancer progression, leading to the development of drugs that could selectively target these abnormalities. Despite the promise of targeted therapies, their success has been limited by the heterogeneity of tumors and the ability of cancer cells to adapt and develop resistance mechanisms.

In recent years, targeted therapies have emerged as a promising alternative, focusing on specific molecular targets associated with cancer progression. These therapies aim to interfere with cancer cell growth and survival pathways, offering the potential for more precise and less toxic treatment options. However, the development of resistance to targeted therapies remains a significant challenge, as cancer cells can adapt and find alternative pathways to sustain their growth and survival.

None of these approaches have provided a comprehensive solution that combines the features described in this disclosure; what is urgently needed are new pathways, treatments and methods for cancers including drug-resistant cancers and treatments for immune compromised individuals.

### BRIEF SUMMARY OF THE INVENTION

The following brief summary presents a simplified summary of the innovation in order to provide a basic understanding of some aspects of the invention. This summary is not an extensive overview of the invention. It is intended to neither identify all key discussion details or critical elements of the invention nor delineate the scope of the invention. Its sole purpose is to present some concepts of the invention in a simplified form as a prelude to the more detailed description that is presented later.

Immunotherapy, which harnesses the body's immune system to fight cancer, has emerged as a promising treatment modality. While immunotherapy has shown remarkable success in certain cancers, such as melanoma and lung cancer, it has not been universally effective. The complexity of the immune system and the ability of cancer cells to evade immune detection continue to pose significant challenges.

One of the promising areas of research disclosed herein involves the manipulation of specific proteins and genes that play a crucial role in the regulation of cell growth and apoptosis. The p73 protein, a member of the p53 family, has garnered attention for its potential in inducing apoptosis in cancer cells. Unlike its counterpart p53, which is often mutated in cancer, p73 remains functional in many tumors, making it an attractive target for therapeutic intervention. Researchers are exploring various strategies to activate p73 and enhance its apoptotic effects, with the aim of developing novel treatments that can overcome the limitations of existing therapies and improve patient outcomes.

Another approach that has gained enablement herein is the modulation of apoptotic pathways to induce cancer cell death. Apoptosis, or programmed cell death, is a natural process that eliminates damaged or unwanted cells. Cancer cells often evade apoptosis, allowing them to survive and proliferate uncontrollably. Strategies to reactivate apoptotic pathways in cancer cells have been explored, including the use of small molecules to modulate proteins involved in apoptosis. Despite these efforts, achieving selective activation of apoptotic pathways in cancer cells without affecting normal cells remains a complex task.

In an introductory example of the technology disclosed herein, a method for treating cancer involves a sophisticated mechanism centered around the activation of the p73 protein, which plays a crucial role in inducing apoptosis in cancer cells. The process begins with the administration of a switcher compound, a p73 protein activator, which is designed to target and modulate the activity of p73 within the cancer cells. This activation is pivotal as it initiates a cascade of molecular events that ultimately lead to the suppression of tumor growth.

Upon administration, the switcher compound binds to the p73 protein, either through ortho-steric or allosteric sites, depending on the nature of the compound. This binding enhances the transcriptional activity of p73, allowing it to effectively regulate the expression of genes involved in cell cycle arrest and apoptosis. One of the key targets of p73 activation is the c-FLIP-L/S gene, which is known to inhibit apoptosis. By reducing the expression of this gene, the switcher compound sensitizes cancer cells to undergo programmed cell death, thereby reducing tumor viability.

In cases where the p53 tumor suppressor protein is inactive, which is common in many cancers, the activation of p73 serves as a compensatory mechanism. The switcher compound effectively restores the apoptotic pathway that is otherwise compromised due to the loss of p53 function. This restoration is achieved through the induction of cell cycle arrest, apoptosis, or differentiation, preventing the uncontrolled proliferation of cancer cells.

The switcher compounds, which can include any suitable small molecules, for example, small molecules such as CB-7587351, RETRA, and Prodigiosin, are meticulously designed to ensure specificity and efficacy in activating p73 (as well as FDA approval). These compounds may also restore wild-type function to mutant p53 proteins, further enhancing their therapeutic potential. The activation of p73 not only regulates the c-FLIP gene but also influences a broader network of genes involved in tumor suppression, thereby exerting a comprehensive anti-cancer effect.

Furthermore, the methods involve identifying human populations that may benefit from this treatment. By analyzing biological samples to determine p73 protein expression or activity levels, researchers can identify subpopulations with low p73 levels. These individuals are likely to respond favorably to the administration of switcher compounds, as the activation of p73 can significantly enhance their cancer cells' susceptibility to apoptosis.

Overall, the activation of p73 by switcher compounds represents a promising therapeutic strategy for cancer treatment. By targeting the molecular pathways that regulate cell death and proliferation, this method offers a targeted approach to combating cancer, particularly in cases where traditional tumor suppressor pathways are compromised. The ongoing research and development of these compounds continue to shed light on their potential to revolutionize cancer therapy, offering hope for more effective and personalized treatment options.

A method for treating cancer may involve obtaining a subject with cancer or at risk of cancer and administering a therapeutically effective amount of a p73 protein activator, known as a switcher compound. The administration of the switcher compound can reduce c-FLIP-L/S gene expression, which may sensitize cancer cells to p73-induced apoptosis, thereby treating or preventing cancer in the subject.

The method may include administering the p73 activating switcher compound in an amount effective to reduce c-FLIP-L/S gene expression, which can sensitize cancer cells to p73-induced apoptosis. In some examples, the method may be applicable where a related p53 tumor suppressor protein is inactive in the cancer cells.

The administration of the switcher compound can compensate for the loss of protein p53′s function by inducing cell cycle arrest, apoptosis, or differentiation, thereby preventing uncontrolled cancer cell growth and tumor development. The switcher compound may activate p73, which transcriptionally regulates the c-FLIP gene and/or its expression.

The switcher compound can act as an orthosteric or allosteric activator of p73. In some examples, the switcher compound may restore wild-type function to a mutant p53 in cancer cells. The switcher compound may also act as a modulator of p73, either in addition to or instead of being an activator. The switcher compound may include small molecules such as CB-7587351,RETRA, Prodigiosin, NSC59984, or combinations thereof, and/or their salts, hydrates, or solvates. The switcher compound may also include small molecules like PRIMA-1, PRIMA-1MET, MIRA-1, STIMA-1, 3-Benzoylacrylic acid, Nutlin-3, RITA, Stictic acid, CP-31398, RETRA, NSC59984, CB-7587351, and/or their salts, hydrates, or solvates.

A composition may comprise a p73 protein activator, which is a switcher compound selected from specific small molecules and/or their salts, hydrates, or solvates. The composition can be effective in a method involving obtaining a subject with cancer and administering a p73 protein activator to reduce c-FLIP-L/S gene expression, sensitizing cancer cells to p73-induced apoptosis. The switcher compound in the composition may also act as a modulator of p73, either in addition to or instead of being an activator. The switcher compound in the composition can restore wild-type function to a mutant p53 in cancer cells. The switcher compound in the composition may activate p73 as an orthosteric or allosteric activator. The switcher compound in the composition can activate p73, which transcriptionally regulates the c-FLIP gene and/or its expression.

A method of identifying a human population may involve obtaining biological samples or data, analyzing them to determine p73 protein expression or activity levels, and identifying a subpopulation with low p73 levels. This subpopulation may benefit from the activation or modulation of p73 by administering a switcher compound to reduce c-FLIP-L/S gene expression, sensitizing cancer cells to p73-induced apoptosis. The switcher compound in this method may include small molecules like CB-7587351, RETRA, Prodigiosin, NSC59984, or combinations thereof, and/or their salts, hydrates, or solvates. The switcher compound in this method may also act as a modulator of p73, either in addition to or instead of being an activator.

The switcher compound in this method can activate p73 as an orthosteric or allosteric activator. The switcher compound in this method may activate p73, which transcriptionally regulates the c-FLIP gene and/or its expression.

Keeping in mind, while contemplating possible combination therapies and the above discussion, as an additional brief summary or to provide discussion points for a brief summary, some example features of the technology disclosed herein can be briefly summarized by the following list of features, any of which can be inter-combined or discussed optionally with any other feature, Figure, Drawing, detail, embodiment, aspect, or example disclosed herein:

Feature 1: A method for treating cancer in a subject in need thereof, the method comprising the steps of: (1) obtaining a subject with cancer, or a subject suspected of having cancer, or a subject who is susceptible to cancer; (2) administering a therapeutically effective amount of a p73 protein activator to the subject, wherein the p73 protein activator is a switcher compound; (3) wherein the administering of the switcher compound is configured to provide a reduction of a c-FLIP-L/S gene expression which is operative to sensitize cancer cells to a p73-induced apoptosis in the cancer cells; thereby treating and/or preventing the cancer in the subject.

Feature 2: The method of feature 1, wherein the p73 activating switcher compound is administered in an amount effective to provide a reduction of c-FLIP-L/S gene expression operative to sensitize the cancer cells to p73-induced apoptosis.

Feature 3: The method of feature 1, wherein a related p53 tumor suppressor protein is inactive in the cancer cells.

Feature 4: The method of feature 1, where the administration of the switcher compound is configured to compensate for a loss of a protein p53′s function by inducing cell cycle arrest, apoptosis or programmed cell death, and/or differentiation, thereby preventing an uncontrolled cancer cell growth and tumor development.

Feature 5: The method of feature 1, wherein the switcher compound activates p73 which transcriptionally regulates the c-FLIP gene and/or an expression of the c-FLIP gene.

Feature 6: The method of feature 1, wherein the switcher compound activates p73 as an orthosteric activator and/or an allosteric activator.

Feature 7: The method of feature 1, wherein the switcher compound is effective to restore wild-type function to a mutant p53 in one or more cancer cells in the subject.

Feature 8: The method of feature 1, further comprising the switcher compound is a modulator of the p73 either in addition to being an activator and/or instead of being an activator.

Feature 9: The method of feature 1, wherein the switcher compound comprises small molecule CB-7587351, small molecule RETRA, small molecule Prodigiosin, small molecule NSC59984, or a combination thereof; and/or a salt, hydrate and/or a solvate thereof.

Feature 10: The method of feature 1, wherein the switcher compound comprises one or more of: small molecule PRIMA-1 or PRIMA-1MET; small molecule MIRA-1; small molecule STIMA-1; small molecule 3-Benzoylacrylic acid; small molecule Prodigiosin; small molecule Nutlin-3; small molecule RITA; small molecule Stictic acid; small molecule CP-31398; small molecule RETRA; small molecule NSC59984; small molecule CB-7587351; and/or a salt, hydrate and/or a solvate thereof.

Feature 11: A composition comprising a p73 protein activator, wherein the p73 protein activator is a switcher compound selected from the group consisting of: 2-[(E)-2-(3,4-dihydroxyphenyl)ethenyl]-1-benzofuran-6-ol (CB-7587351); 2,5-bis(5-hydroxymethyl-2-thienyl)furan (RETRA); 4-methoxy-5-[(Z)-2-pyridin-3-ylvinyl]-1H-pyrrole-2-carbaldehyde (Prodigiosin); and/or 2-[(E)-2-(4-nitrophenyl)ethenyl]-1-benzofuran-5-ol (NSC59984); and/or a salt, hydrate and/or a solvate thereof; wherein the composition is an effective switcher compound in a method comprising the steps of: (1) obtaining a subject with cancer, or a subject suspected of having cancer, or a subject who is susceptible to cancer; (2) administering a therapeutically effective amount of a p73 protein activator to the subject, wherein the p73 protein activator is a switcher compound; wherein the administering of the switcher compound is configured to provide a reduction of a c-FLIP-L/S gene expression which is operative to sensitize cancer cells to a p73-induced apoptosis in the cancer cells; thereby treating and/or preventing the cancer in the subject.

Feature 12: The composition of feature 11, wherein the switcher compound is a modulator of the p73 either in addition to being an activator and/or instead of being an activator.

Feature 13: The composition of feature 11, wherein the switcher compound is effective to restore wild-type function to a mutant p53 in one or more cancer cells.

Feature 14: The composition of feature 11, wherein the switcher compound activates p73 as an orthosteric activator and/or an allosteric activator.

Feature 15: The composition of feature 11, wherein the switcher compound activates p73 which transcriptionally regulates the c-FLIP gene and/or an expression of the c-FLIP gene.

Feature 16: A method of identifying a population of humans wherein an activation and/or a modulation of a p73 protein by an administering of a small-molecule switcher compound or a less than 1000 molecular weight switcher compound will provide a reduction of a c-FLIP-L/S gene expression which is operative to sensitize any cancer cells in the human population to a p73-induced apoptosis in the cancer cells, the method comprising the steps of: (1) obtaining a biological sample from each human in the population and/or obtaining data that has been previously derived from a biological sample from each human in the population; (2) analyzing the biological samples and/or data to determine a level of p73 protein expression and/or activity in the cancer cells; (3) identifying a subpopulation of humans having a low level of p73 protein expression and/or activity in the cancer cells compared to a control; wherein the identified subpopulation of humans is the population wherein an activation and/or a modulation of the p73protein by the administering of the switcher compound will provide the reduction of the c-FLIP-L/S gene expression which is operative to sensitize the cancer cells to the p73-induced apoptosis. Feature 17: The method of feature 16, wherein the switcher compound comprises small molecule CB-7587351, small molecule RETRA, small molecule Prodigiosin, small molecule NSC59984, or a combination thereof; and/or a salt, hydrate and/or a solvate thereof.

Feature 18: The method of feature 16, wherein the switcher compound is a modulator of the p73 either in addition to being an activator and/or instead of being an activator.

Feature 19: The method of feature 16, wherein the switcher compound activates p73as an orthosteric activator and/or an allosteric activator.

Feature 20: The method of feature 16, wherein the switcher compound activates p73which transcriptionally regulates the c-FLIP gene and/or an expression of the c-FLIP gene.

Any of the features, methods and/or details herein can be provided in a kit. While the summary examples disclosed above provide some introduction to embodiments of the invention, other implementations are also contemplated, described, and recited herein. These and other features and advantages will be apparent from a reading of the following detailed description, the example claims, and a review of the associated drawings. It is to be understood that both the foregoing general description and the following detailed description are explanatory only and are not restrictive of aspects as claimed.

### BRIEF DESCRIPTION OF THE DRAWINGS

For the purpose of illustration, certain discernable embodiments of the present invention are shown in the drawings described below. It should be understood, however, that the invention is not limited to the precise arrangements, data, dimensions, and illustrations shown. In the examples of the drawings:

The examples of FIGS. 1A-11 illustrate c-FLIP is a transcriptional target of p73. FIG. 1A shows the overexpression of p73-β increases c-FLIP expression at the protein levels. SW480 cells were infected with recombinant p73-Adenovirus for 48 hr. FIG. 1B shows the overexpression of p73-α increase c-FLIP expression. SW480 cells were transfected with pcDNA-p73-α using lipofectamine. FIG. 1C shows knockdown of p73 reduces c-FLIP protein expression. U2OS cells with p53 wild-type (WT), p53 knockout (KO) or mutant p53 (R175H) were transfected with siRNA targeting p73. The c-FLIP protein expression (A, B and C) was examined by Western Blot. FIG. 1D shows the fragments flanking the c-FLIP promoter used for CHIP-PCR and Luciferase reporter assay. FIG. 1E illustrates endogenous p73 binding to c-FLIP promoter examined by ChIP-PCR assay. FIG. 1F shows a CHIP-PCR assay for the enrichment of c-FLIP promoter bound with Ad-p73 in SW480 cancer cells. The c-FLIP promoters were quantified by real-time PCR using the ChIP-eluted DNA. Data were normalized to the input respectively. Data represent mean±SD. *, P<0.05 (E and F). FIG. 1G illustrates the c-FLIP promoter-luciferase reporter assay in the cells infected with the recombinant adenovirus p73-β for 20 hours. FIG. 1H shows the c-FLIP promoter-luciferase reporter assay in cells transfected with pcDNA-p73-αfor 20 hr. FIG. 1I shows plots of Luciferase activities. Data represent mean±SD. *, P<0.05 (G and H). The data (A-C, and G) represents at least two experiments, and the data (E,F and H) represents one experiment. Data are expressed as mean±SD. *, P<0.05

The examples of FIGS. 2A-2F illustrate depletion of c-FLIP sensitizes cancer cells to p73-induced cell death. FIG. 2A shows sub-G1 flow cytometric assay in SW480 cells that were infected with Ad-p73 for 48 hr. FIG. 2B shows cell cycle analysis in SW480 cells infected with Ad-p73 for 48 hr (A) by Flowjo. FIG. 2C shows sub-G1 assay in SW480 cells infected with Adp73 for 48 and 72hours. FIG. 2D shows sub-G1 assay in U2OS-p53KO cells with knockdown of c-FLIP and overexpression of p73-β. c-FLIP was transiently knocked down by siRNA and p73-8 was overexpressed with adenovirus infection for 30 hr. FIG. 2E shows cleaved-PARP protein in cells with knockdown of c-FLIP and overexpression of p73 protein after Ad-p73 infection for 30 hr. FIG. 2F shows Western Blot assay for protein levels in SW480 cells with knockdown of c-FLIP isoforms followed by p73a transfection. G. Real time-Glo Annexin V assay. The cells were transfected with siRNA as indicated, followed with AdLac or Adp73 infection for 44 hours. The data (A,B and C) represents a combination of two independent experiments. The data (D and E) represents at least two independent experiments. The data (F and G) represent one experiment. Data are expressed as mean±SD. *, P<0.05.

The examples of FIGS. 3A-3E illustrate p73 induces cell death via extrinsic apoptosis in c-FLIP-deficient cancer cells. FIG. 3A shows sub-G1 analysis in U2OS-p53KO cancer cells with knockdown of caspase-8 and caspase-9. The cells were transfected with the siRNAs to knockdown caspase-8, caspase-9 or c-FLIP-s+L expression, followed with Ad-p73 or AdLacZ infection for 72 hr. FIG. 3B shows cleaved PARP in U2OS-P53KO cells with knockdown of caspase-8 and 9. Caspase-8, caspase-9 and c-FLIP-s+L were knocked down by siRNA in the cells followed with Ad-p73 or AdLacZ infection for 30 hr. FIG. 3C shows cleaved PARP in U2OS-P53KO cells with knockdown of DR5 and FADD. U2OS-P53KO cells were transfected with siRNA to knock down DR5 and FADD expression, followed with Ad-p73 or AdLacZ infection for 30 hr. FIG. 3D shows cleaved PARP in tumor cells with knockdown of PUMA and BID. U2OS-P53 KO cells were transfected with siRNA to knock down PUMA and BID expression, followed by Ad-p73 or AdLacZ infection for 30 hr. The data (A-D) represents two experiments. Data are expressed as mean±SD. *, P<0.05. FIG. 3E shows a table/heatmap targeting from >2 to <0.5.

The examples of FIGS. 4A-4H illustrate identification of small molecule “switch compound” CB-7587351 as a c-FLIP degrader. FIG. 4A shows an example molecular structure of “switch compound” CB-7587351. FIG. 4B shows c-FLIP protein levels in tumor cells treated with “switch compound” CB-7587351. FIG. 4C shows mRNA levels of c-FLIP in cells treated with CB-7587351. FIG. 4D shows protein levels of c-FLIP in SW480 cells with overexpression of recombinant p73-8, followed by CB-7587351 treatment for 8 hr. FIG. 4E shows protein levels of c-FLIP in cells treated with CB-7587351 and MG132. FIG. 4F and FIG. 4G show c-FLIP protein levels in SW480 cell with knockdown of ITCH or caspase 8. The ITCH or Caspase 8 was knocked down with siRNA for 48 hr, followed with CB-7587351 treatment for 8 hr. FIG. 4H show c-FLIP protein levels in the ChIP-knockdown tumor cells. ChIP was knocked down by siRNA in SW480 cells, followed by CB-7587351 treatment for 8 hr. The data (B) represents at least two experiments related to 8-and 16-hour time points. The data (C, D and G) represents one experiment. The data (E and F) represents two experiments about ITCH. Data are expressed as mean±SD. *, P<0.05.

The examples of FIGS. 5A-5F show CB-7587351 activates p73-dependent pro-apoptotic transcriptional target genes. FIG. 5A shows PG13-bioluminescent assay in cancer cells treated with CB7587351 at different time points. FIG. 5B shows the protein levels of p53 targets in different cancer cells treated with CB-7587351 for 16 hr. FIG. 5C shows mRNA levels of p53 target genes in SW480 cells treated with CB-7587351 for 3 hr. mRNAs were examined by real time PCR. Data was normalized to GAPDH expression and plotted relative to cells treated with DMSO as control. Data are expressed as mean±SD. *, P<0.05 vs. control. FIG. 5D shows PG13-luciferase reporter assay in cells with overexpression of p53 or p73. Cells were infected with Ad-p53 or Ad-p73, followed by CB-7587351 treatment for 16 hr. Relative bioluminescence was normalized to those of DMSO treatment in the cells without the infection. Data are expressed as mean±SD. *P<0.05. FIG. 5E shows the expression of Adp53 and Adp73 in the cells (D). FIG. 5F shows protein levels of p53 targets in p73 knockdown DLD-1 cells treated with CB-7587351 for 8 hr. Compare the PG13-Luciferase reporter assay. Cancer cells were transfected with siRNA targeting p73, followed with CB7587351 (UM) treatment. The relative Bioluminescence levels were normalized to those of siCtrl+DMSO. The data (A,C,D, E and F) represent one experiment. The data (B) represents at least two independent experiments. The data in the Luciferase assay represents four independent experiments. Data are expressed as mean±SD. *, P<0.05.

The examples of FIGS. 6A-6I show pro-apoptotic effects of CB-7587351 on cancer cells. FIG. 6A shows cell viability assay. Cells were treated with CB-7587351 for 48 hr. FIG. 6B shows cell cycle profiles in the cells treated with CB07587351 for 48 hr. FIG. 6C shows colony formation assay as indicated. FIG. 6D shows sub-G1 assay in p73-knockdown DLD-1 cells treated with CB-7587351 for 72 hr. FIG. 6E shows protein analysis of cleaved PARP and cleaved caspase 3 in p73-knockdown DLD-1 cells treated with CB-7487351 for 48 h. FIG. 6F shows sub-G1 assay in cancer cells infected with Ad-p73 or AdLacZ and treated with CB-7587351 for 48 hr. FIG. 6G shows annexin-luciferase assay in cells treated with Adp73 and CB7587351 for 24 hours. The data (A, B, C and G) represent one experiment. The data (D, E and F) represent two experiments. Data are expressed as mean±SD. *, P<0.05. FIG. 6I shows supporting microscope images (100×).

The examples of FIGS. 7A-7F show CB-7587351 induces cell death via the extrinsic apoptotic pathway. FIG. 7A shows cleaved caspase-3 in the cells treated with CB-7587351.FIG. 7B shows caspase-3/7 activity in DLD-1 cancer cells treated with CB-7587351 for 36 hours. FIG. 7C shows cell cycle profiles in p53-mutant cancer cells treated with CB-7587351 and pan-caspase inhibitor z-VAD-FMK. FIG. 7D shows cell cycle profiles in p53 wild-type and p53-null cancer cells treated with CB-7587351 and pan-caspase inhibitor z-VAD-FMK. FIG. 7E shows cleaved PARP in cancer cells with knockdown of caspase-8 or caspase-9, followed by CB-7587351 treatment. FIG. 7F shows bioluminescent Annexin-V assay in HT29 cells. The cells transfected with siRNA to knock down caspase 8 or caspase 9, followed with CB-7587351 (UM) treatment for 41 hours. The data (A, C, D and F) represents one experiment. The data (B and E) represents two independent experiments. Data are expressed as mean±SD. *, P<0.05.

The examples of FIGS. 8A-8E show how combinational treatment of TRAIL and CB-7587351 in cancer cells reveals synergy. FIG. 8A shows cell viability assay. FIG. 8B shows the synergy assay (A). FIG. 8C shows a protein assay of cleaved PARP in tumor cells treated with CB-7587351 and TRAIL. FIG. 8D shows cleaved PARP in HT29 cells with knockdown of caspase-8,followed by the combinational treatment. FIG. 8E shows and example schematic of the mechanism of p73 priming of extrinsic apoptosis in cancer cells. The data (A,B and D) represents one experiment, and the data (C) represents at least two independent experiments.

The examples of FIGS. 9A-9B show p73 increases c-FLIP expression. FIG. 9A shows Saos2 cells were transfected with pcDNAP73a by lipofectamine. FIG. 9B shows p73 expression was knocked down by siRNA in p53-mutant cancer cells and the recombinant p73b was overexpressed by adenovirus infection in SW480 cells.

The examples of FIGS. 10A-10C show knockdown of c-FLIPL/S isoforms sensitizes tumor cells to recombinant p73-b induced cell death. FIG. 10A shows c-FLIP-L or c-FLIP-S were knocked down in p53 wild-type U2OS osteosarcoma cells. FIG. 10B shows c-FLIPL/S expression was knocked down in p53 mutant-HT29 cells. FIG. 10C shows c-FLIP-L or c-FLIP-S were knocked down in mutant p53 (R175H) expressing U2OS.

The examples of FIGS. 11A-11D show p73a and p73b induce extrinsic apoptosis in c-FLIP knockdown cancer cells. FIG. 11A shows sub-G1 analysis in SW480 cells with Ad-p73b infection for 48 hr and 72 hr. FIG. 11B shows sub-G1 analysis in HCT116 p53-null cancer cells with knockdown of caspase-8 and caspase-9. Tumor cells were transfected with siRNA to knockdown caspase-8, caspase-9 or c-FLIP expression, followed with Ad-p73b infection for 30 hr. FIG. 11C shows c-FLIP-L or c-FLIP-S were knocked down in SW480 cells, followed by p73a overexpression. FIG. 11D shows knockdown of caspase-8, caspase-9 and c-FLIP in SW480 cells, followed with pcDNA-p73a transfection.

The examples of FIGS. 12A-12C show p73-b induces extrinsic apoptosis in cells with knockdown of c-FLIP. FIG. 12A shows knockdown of caspase-8, caspase-9 and c-FLIP in p53 KO cells. FIG. 12B shows knockdown of caspase-8, caspase-9 and c-FLIP in p53-mutant cancer cells. FIG. 12C shows knockdown of caspase-8, caspase-9 and c-FLIP in p53 wild-type cancer cells. p73b was overexpressed in tumor cells (A, B, and C) by adenovirus infection for 16 hr.

The examples of FIGS. 13A-13C show DR5 is required for p73 to induce apoptosis. FIG. 13A shows DR5 and FADD were knocked down in P53 KO cancer cells. FIG. 13B shows DR5 and FADD were knocked down in U2OS. FIG. 13C shows bid and PUMA were knocked down in U2OS cells. p73b was overexpressed in the cells (A-C) by adenovirus infection.

FIG. 14 is an example flowchart illustrating a method in step for treating cancer in a subject, according to an embodiment.

FIG. 15 is an example flowchart illustrating a method for identifying a population of humans wherein an activation and/or a modulation of a p73 protein by administering a small-molecule switcher compound may provide a reduction of a c-FLIP-L/S gene expression, which is operative to sensitize any cancer cells in the human population to a p73-induced apoptosis in the cancer cells, according to an embodiment.

It should be understood that while illustrations can sometimes be used in the example figures above to describe different embodiments and different aspects of the technology, any aspect from any figure can be optionally inter-combined with an aspect, feature or detail from any other figure or text. Any example disclosed herein can be inter-combined with any other. All trademarks, images, likenesses, words, and depictions that could be construed in the drawings and the disclosure are plainly in fair use and are provided solely for the purposes of illustration of the invention in view of an urgent need to prevent injuries and to treat subjects as further discussed in more detail below.

### DETAILED DESCRIPTION OF THE INVENTION

The subject innovation is now described, in some examples with reference to the drawings, wherein examples can used to refer to the aspects of the breadth of concepts of the invention. In the following description, for purposes of explanation, specific details are set forth in order to provide a thorough understanding of the present invention. It may be evident, however, that the present invention may be practiced without these specific details. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention can be determined by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, a “range” may be provided. A statement may include “in the range from about A to about B”. All points from A to B are subsumed by the range, and all those points can define preferred ranges. Within said range, any range subsumed therein means any range that is within the stated range. Endpoints within the range can define a new range. For example, the following are all subsumed within the range of about 10 to about 50. 10 to 20; 15 to 35; 23 to 40; or 50 to 31; or any other range or set of ranges within the stated range. As such, within the range any set of endpoints subsumed therein can be used as an exemplary range.

As used herein, the term “comprising” means that other elements can also be present in addition to the defined elements presented. The use of “comprising” indicates inclusion rather than limitation. Any method described herein can be claimed and/or described as a composition and vice versa.

The term “consisting of” as it is known in the practice refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. In specific examples, “consisting essentially of” can be explained herein for each example or can be defined broadly, for example, by stating that an administration to a subject (in a method herein) does not include any other active pharmaceutical ingredient or therapeutic agent in addition to the one specified. In another example, the term “consisting essentially of” can be utilized to indicate a nanocarrier and a therapeutic agent with no other ingredients that are listed in a claim and yet including any other ingredients that are not specifically listed.

The term “statistically significant” or “significantly” refers to statistical significance and generally means a two-standard deviation (2SD) or greater difference. The term “feature” and the term “detail” can be interchanged with a “claim”. Any list of features, details, examples, embodiments, and/or aspects herein can be placed into a “claim”.

As used herein, the term “subject” refers to a mammal, bird, or the like, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), racing mammals, laboratory animals (e.g., mice, rats, rabbits, etc.), but are not so limited. Subjects include human subjects. The human subject may be a pediatric, adult, or a geriatric subject. The human subject may be of either sex. In another example, the term “subject” can refer to a connective tissue culture, and the methods disclosed herein, while claimed towards subjects, contemplate use in the laboratory in synthetic tissue(s). As used herein, a female cell can refer to a cell with 2X chromosomes; a male cell can refer to a cell with 1X and 1Y chromosome.

As used herein, the terms “effective amount” and “therapeutically effective amount” include an amount sufficient to modulate a treatment or prevent or ameliorate a manifestation of disease or medical condition, such as a connective tissue condition or a risk of a connective tissue injury. Such a condition (or risk) may not be readily discernable and may take years, statistical analysis, and/or machine learning to determine a prevention, treatment, or amelioration. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the condition and on the characteristics of the individual, such as general health, age, sex, body weight and tolerance to drugs. It will also depend on the degree, severity and type of condition. The skilled artisan will be able to determine appropriate dosages depending on these and other factors. The compositions can also be administered in combination with one or more additional therapeutic compounds.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, manage, modulate, slow down or stop the progression or severity of a symptom or condition. The term “treating” includes reducing or alleviating at least one adverse effect (undesirable characteristic) or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a condition or decay, delay or slowing of a progression and/or risk of injury, and an increased lifespan/enjoyment as compared to that expected in the absence of treatment.

As used herein, the term “long-term” administration means that the therapeutic agent or drug is administered for a period of at least 12 weeks. The therapeutic agent or drug may refer to a formulation, composition, or agent. The formulation can be changed to a fresh formulation during administration. This includes that the therapeutic agent or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by any technique known in the art that is subsequently applied to a subject, topical application, absorption, injection, ingestion, transfusion, implantation or transplantation. In an example embodiment, compositions are applied as a tablet or drug in capsule. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subdermal, subcuticular, intraarticular, subcapsular, subarachnoid, intraspinal and intrasternal injection and infusion. It is known in the art that therapeutic agents can be rapidly deployed through the skin and directly into joint/ligaments by use of DMSO (dimethyl sulfoxide) as a carrier solvent applied (with the therapeutic agent) to the skin near to or surrounding a joint. While DMSO is rarely used anymore for these purposes because of its nature as a universal solvent and its tendency to carry any residual chemicals present on the skin into the bloodstream (along with the intended agent), the technology contemplates such uses. In one contemplated embodiment, the compositions contemplated herein are administered to a subject by direct injection into a tissue, lymph node, or site of treatment. In another example, administration is provided in the form of a natural product, vitamin, supplement, food, aerosol, inhalation, vapor, or drink. Formulations disclosed herein can be ready made or require mixing just before administration.

Any of the methods disclosed herein can be carried out in part or completely by including a dietary change, a food, natural product, precursor, or prodrug of a therapeutic agent. As used herein, a precursor or a prodrug is intended to encompass compounds or therapeutic agents which, under physiologic conditions, are converted into the therapeutically active agents of the present invention (e.g., a compound for any of the present claims or features). A common method for making a prodrug is to include one or more selected moieties which are hydrolyzed under physiologic conditions to reveal the desired molecule. In other embodiments, the prodrug is converted by an enzymatic activity of the host subject. For example, esters or carbonates (e.g., esters or carbonates of alcohols or of carboxylic acids) are preferred prodrugs of the present invention. In certain embodiments, some or all of the small-molecule chemical structures selected from this disclosure can be replaced with the corresponding suitable prodrug, for example, wherein a hydroxyl in the parent compound is presented as an ester or a carbonate or carboxylic acid present in the parent compound is presented as an ester. A common method of making a precursor/prodrug that can be used herein is to use a carrier/nanocarrier (e.g., mesoporous silica particles). The precursor/prodrug can be released from a carrier to form the active therapeutic agent. A precursor or prodrug can be metabolized to the active parent compound (therapeutic agent) in vivo (e.g., the ester is hydrolyzed to the corresponding hydroxyl, or carboxylic acid). No argument can be made that the term “prodrug” is not enabled herein based on an assertion that actual prodrugs were not made and tested.

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms: “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the term: “small molecule” refers to a molecule that has a molecular weight <1000. As used herein, the term: “large molecule” refers to a molecule that has a molecular weight >1000, and the term includes biologics such as the examples of oligonucleotides, peptides, antibodies, linkers, oligosaccharides, polymers, DNA chains, and RNA chains. The term: “therapeutic agent” may refer to small molecule, element, large molecule, biologic, formulation, composition, agent, or a combination thereof.

In some embodiments, in drug discovery, a “hit” can refer to a compound that exhibits the desired activity in a high-throughput screening (HTS) assay, indicating potential as a starting point for further development. ChemBridge is a provider of high-quality screening compounds and libraries used in HTS to identify such hits. In one example, a “ChemBridge library hit” specifically denotes a compound from ChemBridge's collection that has shown promising activity in a screening assay. PMC+2Drug Target Review+2PMC+2chembridge.com+1chembridge.com+1

The significance of identifying a hit from the ChemBridge library lies in the quality and diversity of their compounds. ChemBridge offers over 1.3 million small molecule screening compounds, including lead-like and drug-like compounds, macrocycles, and fragments. These compounds are designed to cover unique chemical space and are synthesized based on proprietary schemes, ensuring novelty and diversity. Each compound undergoes rigorous quality control, including identity confirmation and purity assessment, making them reliable candidates for drug discovery projects. chembridge.com+2chembridge.com+2chembridge.com+2

Securing a hit from such a reputable library means that the compound has a well-characterized profile and is part of a diverse chemical space, which can be advantageous for developing novel therapeutics. Furthermore, ChemBridge's experience and track record in providing compounds for successful HTS campaigns add to the credibility and potential of hits identified from their libraries. chembridge.com+1chembridge.com+1

In summary, a “ChemBridge library hit” signifies a compound from ChemBridge's extensive and well-curated collection that has demonstrated promising activity in a screening assay, serving as a valuable starting point for drug development efforts.

The technology disclosed herein is not limited by the specific small molecules mentioned.

### Pharmaceutical Compositions

The compositions and methods of the present invention may be utilized to prevent a need for other treatment, to provide benefit when other treatment(s) fail, or to treat an individual in need thereof. In some embodiments, the individual is suspected of needing treatment. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. A compound can represent a combination therapy herein. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. Compositions can be in gas forms. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible compositions employed in pharmaceutical formulations.

A pharmaceutical composition (preparation) can be administered to a subject by any of a number of routes of administration including, for example, orally, for example, drenches as in aqueous or non-aqueous solutions or suspensions, tablets, capsules including sprinkle capsules and gelatin capsules, boluses, powders, granules, pastes for application to the tongue; absorption through the oral mucosa (e.g., sublingually); subcutaneously; transdermal administration (for example as a patch applied to the skin); and topically (for example, as a cream, ointment or spray applied to the skin). The compound may also be formulated for inhalation. Inhalation can include inhalation of a liquid (droplets or aerosol). Inhalation can include a micronized powder adhered to carrier particles or can be without carrier particles. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, all of which are incorporated herein by reference in their entireties, as well as in patents cited therein.

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention or are used as would be understood by an ordinary person.

### Tumor Suppressor P73 Transcriptionally Regulates C-Flip to Impede its Priming of Extrinsic Apoptosis While an Example Switcher Compound, CB-7587351, Degrades C-Flip Protein

The tumor suppressor p73 is a member of the p53 family and transcriptionally activates multiple p53-targets involved in cell cycle regulation and apoptosis. In addition to pro-apoptotic signaling, outcomes of p73 activation include cell survival signals. Thus, p73 activity and targets may provide insight in cell fate outcomes between cell survival and apoptosis following cellular stress. Our discovery of p73 transcriptional upregulation of c-FLIP provides a promising strategy for depleting c-FLIP to improve antitumor efficacy of p73-targeting cancer therapy for p53-mutant tumors. Targeting c-FLIP-L/S protein degradation with the “switcher compound” approach we describe leads cancer cells towards a p73-induced extrinsic apoptotic cell fate.

In some embodiments herein, we exploit that cellular FLICE inhibitory protein (c-FLIP), a master anti-apoptotic factor, is a transcriptional target of p73. The activation of p73 (alpha and beta isoforms) transcriptionally upregulates c-FLIP-L/S expression in cancer cells. The cell fate decision following p73 activation is determined by the adjustment of the balance of outcomes of p73 activation between p73-induced pro-apoptotic signaling and c-FLIP-L/S expression in cancer cells. As an example, we identified a small-molecule CB-7587351 as a switcher compound that alters p73 activation outcomes through c-FLIP-L/S protein degradation. According to some aspects, small molecule CB-7587351 works as a “switcher compound” to adjust the outcome of p73 signaling through degradation of c-FLIP and activation of the p73 pathway in cancer cells for cancer therapy. In some embodiments, the technology disclosed herein develops a new strategy for harnessing c-FLIP-L/S depletion to increase the antitumor efficacy of targeting p73 in TP53-deficient cancer cells for cancer therapy.

In some embodiments, the technology herein provides a method for treating cancer in a subject in need thereof, the method comprising the steps of: (1) obtaining a subject with cancer or a subject suspected of having cancer or a subject who is susceptible to cancer; (2) administering a therapeutically effective amount of a p73 protein activator to the subject, wherein the p73 protein activator is a switcher compound; (3) wherein the administering of the switcher compound is configured to provide a reduction of a c-FLIP-L/S gene expression which is operative to sensitize cancer cells to a p73-induced apoptosis in the cancer cells; thereby treating and/or preventing cancer in the subject.

Cancer is a major problem in the United States; cancer being the second leading cause of death; thus, the technology herein provides a new solution for treating the problem of cancer. An example of the novelty of the technology is that using a p73 protein activator, specifically a switcher compound, to reduce c-FLIP-L/S gene expression, is enhancing p73-induced apoptosis.

According to some aspects, treating cancer by activating the p73 protein involves leveraging its tumor suppressor function to target and kill cancer cells, especially when the related p53 tumor suppressor protein is inactive. p73 can compensate for p53′s loss by inducing cell cycle arrest, apoptosis (programmed cell death), and differentiation, thereby preventing uncontrolled cell growth and tumor development.

Some examples include p73 as a tumor suppressor: p73, a protein closely related to p53, plays a crucial role in preventing cancer development. It helps regulate cell growth, division, and death, acting as a “guardian” to prevent the uncontrolled proliferation of cancer cells.

Complementary role to p53: while p53 is mutated in many cancers, p73 is often overexpressed or preserved, allowing it to take on some of p53′s tumor-suppressing functions. This makes p73 a valuable target for cancer therapies, especially in cases where p53 is dysfunctional.

Example mechanism of action: p73 can induce cell cycle arrest (stopping cell division), apoptosis (programmed cell death), and differentiation (specialization of cells), all of which help to prevent cancer development.

The technology herein provides ways to activate p73 in cancer cells to trigger its tumor-suppressive effects. This involves using the small-molecules drugs or other interventions to increase p73 expression or activity.

Dual role: While p73 primarily acts as a tumor suppressor, it can also have a dual role in certain contexts. It may promote tumor growth by influencing the immune system and promoting angiogenesis (new blood vessel formation), highlighting the complexity of its actions.

Targeting the p73 pathway: herein are disclosed various ways to manipulate the p73 pathway, including upstream regulators and interactors, to induce p73 accumulation and enhance its tumor-suppressive function. This involves identifying and developing small molecules that can directly activate p73 or interfere with its inhibitors.

This disclosure provides a method for treating cancer in a subject by administering a therapeutically effective amount of a p73 protein activator, which may be a switcher compound. The administration of the switcher compound can lead to a reduction in c-FLIP-L/S gene expression, which may sensitize cancer cells to p73-induced apoptosis. The method may compensate for the loss of protein p53′s function, potentially preventing uncontrolled cancer cell growth and tumor development. The switcher compound can activate p73 as an orthosteric or allosteric activator, enhancing p73 activity. The method may include compositions comprising small molecules such as CB-7587351, RETRA, Prodigiosin, and NSC59984, which can be effective in treating cancer. The disclosure may also involve identifying a population of humans suitable for treatment based on p73 protein expression levels.

In the context of treating cancer, a method may involve the administration of a therapeutically effective amount of a p73 protein activator, which may be a switcher compound, to a subject. This administration may be configured to activate p73 as an orthosteric activator and/or an allosteric activator, potentially enhancing p73 activity. The activation of p73 may lead to a reduction in c-FLIP-L/S gene expression, which may sensitize cancer cells to p73-induced apoptosis. This sensitization may be significant in treating and/or preventing cancer in the subject. The switcher compound may be administered in a manner that provides a reduction of c-FLIP-L/S gene expression, thereby sensitizing the cancer cells to apoptosis. The method may also involve the use of specific small molecules, such as CB-7587351, RETRA, Prodigiosin, or NSC59984, which may serve as effective switcher compounds. These small molecules may be selected for their ability to activate p73 and modulate its activity, thereby contributing to the overall therapeutic strategy. The approach may be designed to treat cancer by leveraging the potential of p73 protein activators to induce apoptosis in cancer cells, offering a targeted method for cancer treatment.

In the context of treating cancer, the function of protein p53 may be compensated for when its activity is lost. This compensation may prevent uncontrolled cancer cell growth and tumor development. The restoration of wild-type function to a mutant p53 may also be achieved, potentially restoring normal function in cancer cells. The actions associated with protein p53 and mutant p53 may be correlated with the actions of compensating for a loss of protein p53′s function and restoring wild-type function to a mutant p53. These actions may be relevant in the context of the method for treating cancer. The switcher compound, which may act as a p73 protein activator, may be administered to the subject to provide a reduction of c-FLIP-L/S gene expression, thereby sensitizing cancer cells to p73-induced apoptosis. This process may be significant in treating and/or preventing cancer in the subject. The switcher compound may also activate p73 as an orthosteric activator and/or an allosteric activator, enhancing p73 activity. The method may involve administering a therapeutically effective amount of the p73 protein activator to the subject, which may be configured to provide a reduction of c-FLIP-L/S gene expression. This reduction may sensitize cancer cells to p73-induced apoptosis, thereby treating and/or preventing cancer in the subject. The actions of compensating for a loss of protein p53′s function and restoring wild-type function to a mutant p53 may be integrated into the overall method for treating cancer, providing a comprehensive approach to addressing the challenges associated with cancer treatment.

In the context of the system, the modulation of the p73 protein may be considered as a potential action to alter p73 activity. This modulation may involve the use of a switcher compound, which can be administered to the subject. The switcher compound may serve as a p73 protein activator, potentially acting as an orthosteric or allosteric activator. The administration of the switcher compound may be configured to provide a reduction of c-FLIP-L/S gene expression, which may sensitize cancer cells to p73-induced apoptosis. This process may be aimed at treating and/or preventing cancer in the subject. The modulation of p73 may also be considered in the context of compensating for a loss of protein p53′s function, which may prevent uncontrolled cancer cell growth and tumor development. Additionally, the modulation may restore wild-type function to a mutant p53, thereby restoring normal function in cancer cells. The switcher compound may comprise small molecules such as CB-7587351, NSC59984, RETRA, or Prodigiosin, which may provide specific compounds for treatment. The modulation of p73 may be a step in the method for treating cancer, as it may enhance p73 activity and sensitize cancer cells to apoptosis. The modulation may also be considered in identifying a population of humans suitable for treatment, where the activation and/or modulation of p73 by the switcher compound may provide a reduction of c-FLIP-L/S gene expression, sensitizing cancer cells to p73-induced apoptosis.

According to an embodiment, a population of humans may be identified to determine suitable candidates for treatment. This identification may involve obtaining a biological sample from each human in the population and/or obtaining data previously derived from a biological sample. The analysis of these biological samples and/or data may be conducted to determine a level of p73 protein expression and/or activity in the cancer cells. A subpopulation of humans having a low level of p73 protein expression and/or activity in the cancer cells compared to a control may be identified. The identified subpopulation of humans may be the population wherein an activation and/or a modulation of the p73 protein by the administering of the switcher compound may provide the reduction of the c-FLIP-L/S gene expression, which may be operative to sensitize the cancer cells to the p73-induced apoptosis. The switcher compound may comprise small molecules such as CB-7587351, RETRA, Prodigiosin, or NSC59984, or a combination thereof, and/or a salt, hydrate, and/or a solvate thereof. The switcher compound may also be a modulator of the p73 either in addition to being an activator and/or instead of being an activator. The activation of p73 may occur as an orthosteric activator and/or an allosteric activator, which may transcriptionally regulate the c-FLIP gene and/or an expression of the c-FLIP gene.

In the context of the system, small molecules such as CB-7587351, NSC59984, RETRA, and Prodigiosin may be utilized as switcher compounds. These small molecules can be integral in the treatment process by providing specific compounds for therapeutic intervention. The administration of these small molecules may be configured to activate the p73 protein, which can be achieved through orthosteric or allosteric activation. This activation may lead to the transcriptional regulation of the c-FLIP gene, potentially resulting in a reduction of c-FLIP-L/S gene expression. Such a reduction may sensitize cancer cells to p73-induced apoptosis, thereby contributing to the treatment and/or prevention of cancer in the subject. The switcher compounds may also be effective in restoring wild-type function to a mutant p53 in cancer cells, which can be significant for restoring normal cellular function. The method may involve obtaining a subject with cancer or a subject suspected of having cancer, followed by the administration of a therapeutically effective amount of the p73 protein activator. The process may be designed to compensate for the loss of protein p53′s function by inducing cell cycle arrest, apoptosis, or differentiation, thereby potentially preventing uncontrolled cancer cell growth and tumor development. The switcher compounds may be administered in a manner that modulates the p73 protein, altering its activity to achieve the desired therapeutic outcome. The selection of specific small molecules for treatment may be based on their ability to provide a reduction in c-FLIP-L/S gene expression, which is operative in sensitizing cancer cells to apoptosis. The overall approach may be tailored to the specific needs of the subject, with the potential to enhance the efficacy of cancer treatment through the strategic use of these small molecules.

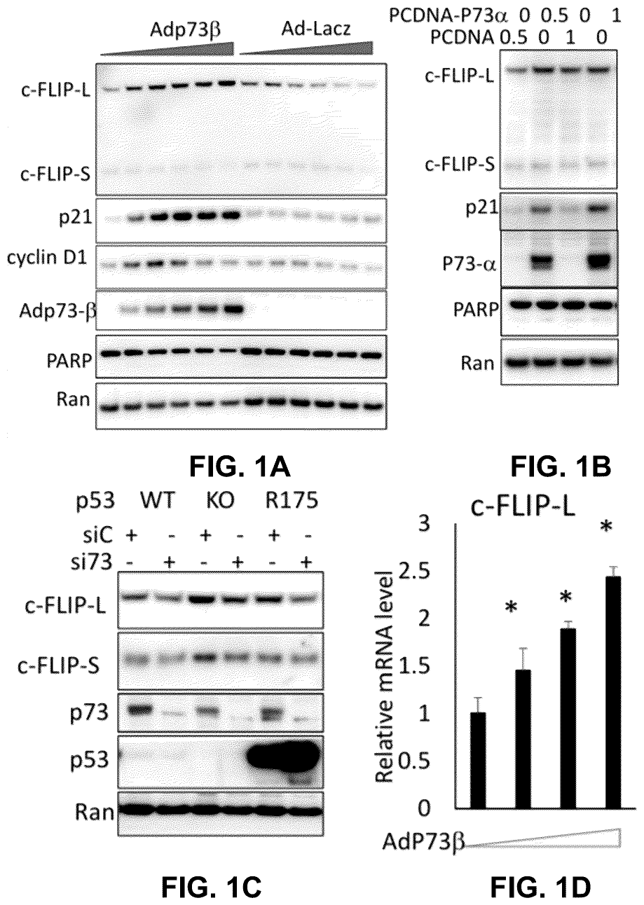

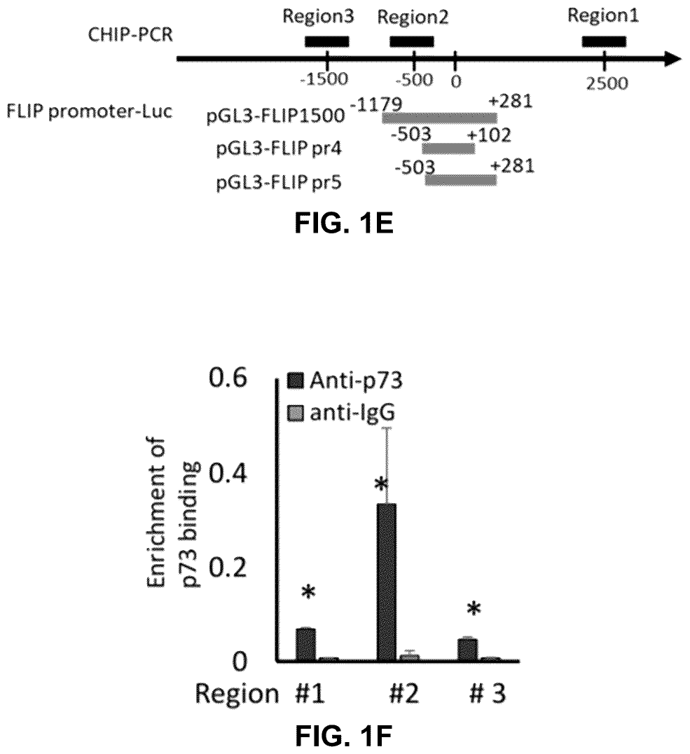

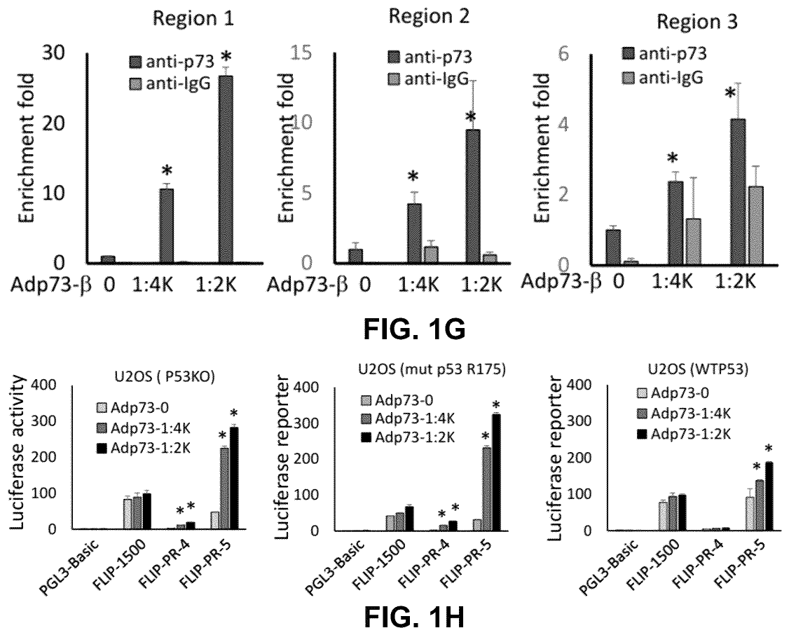

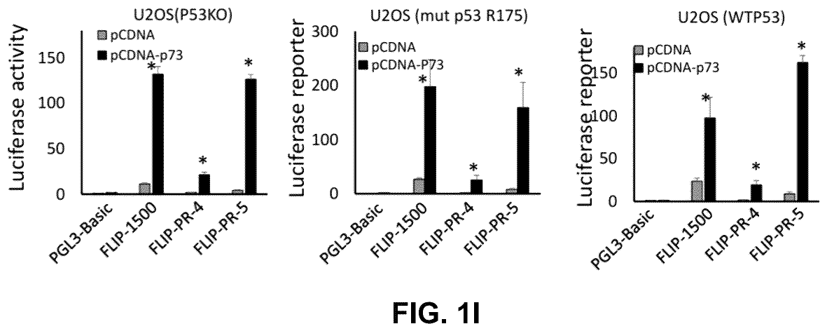

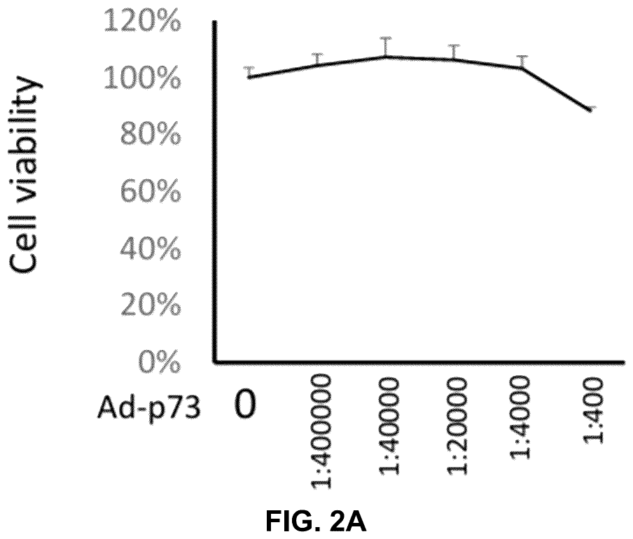

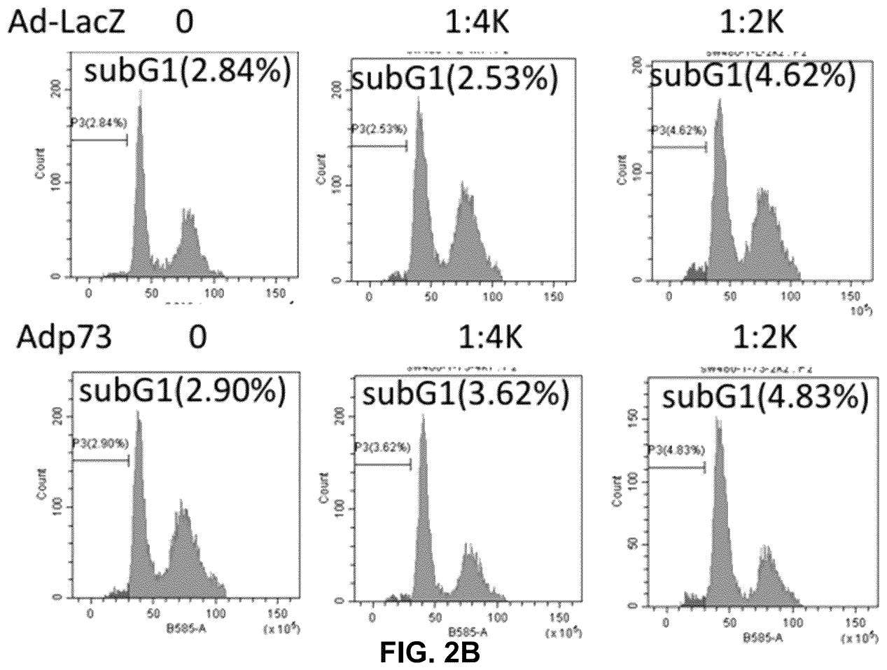

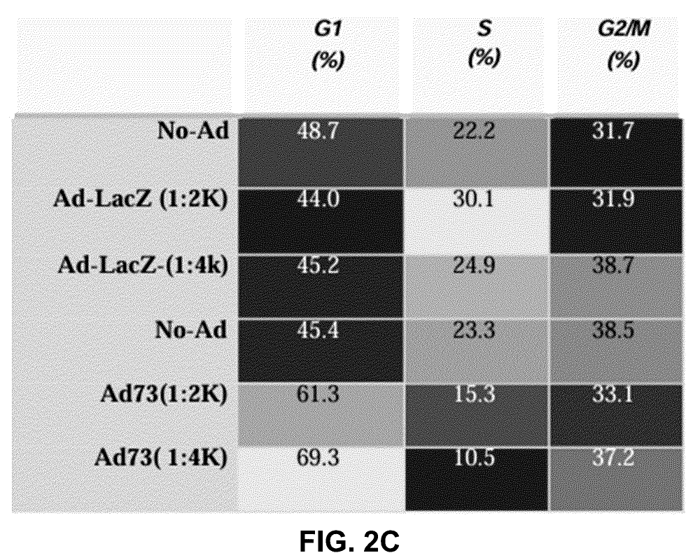

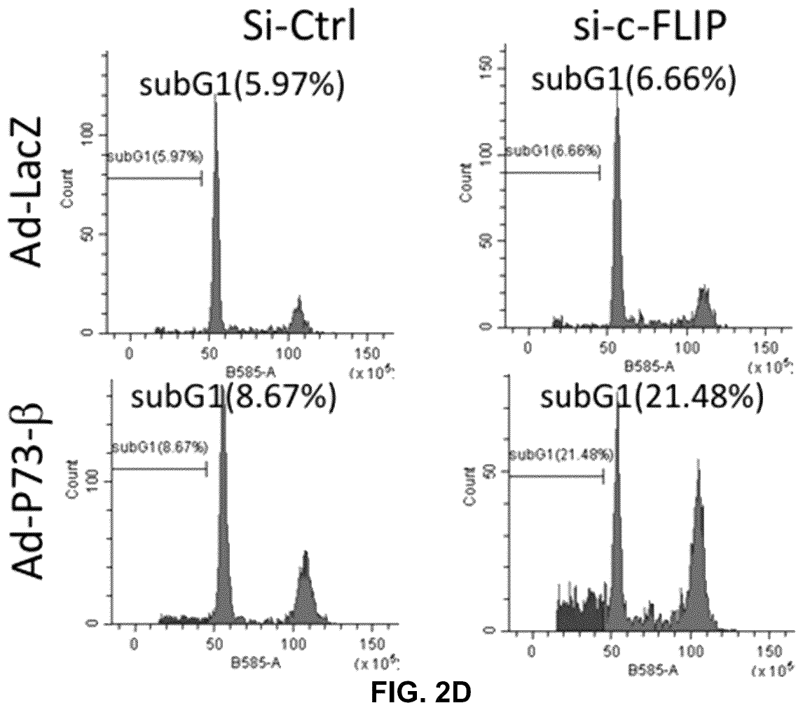

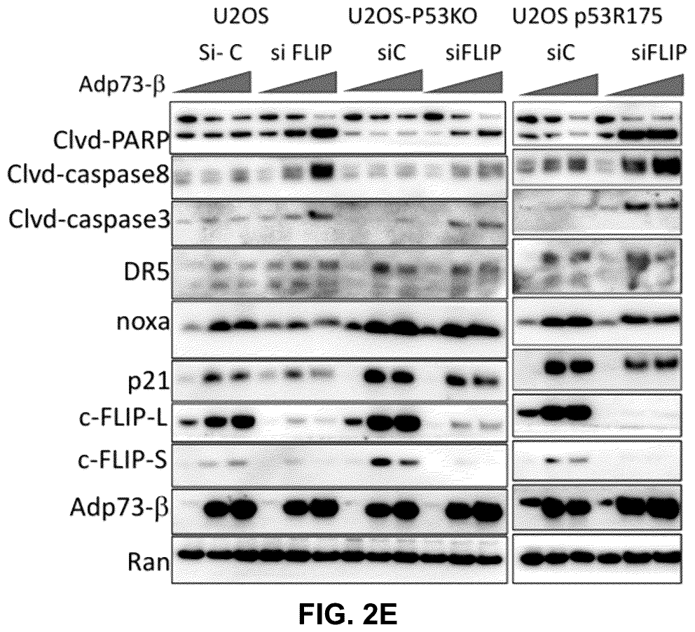

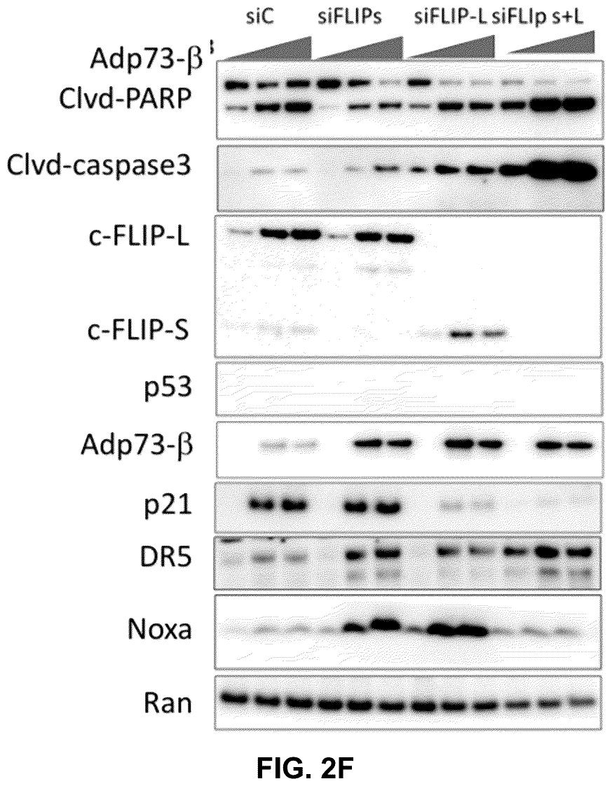

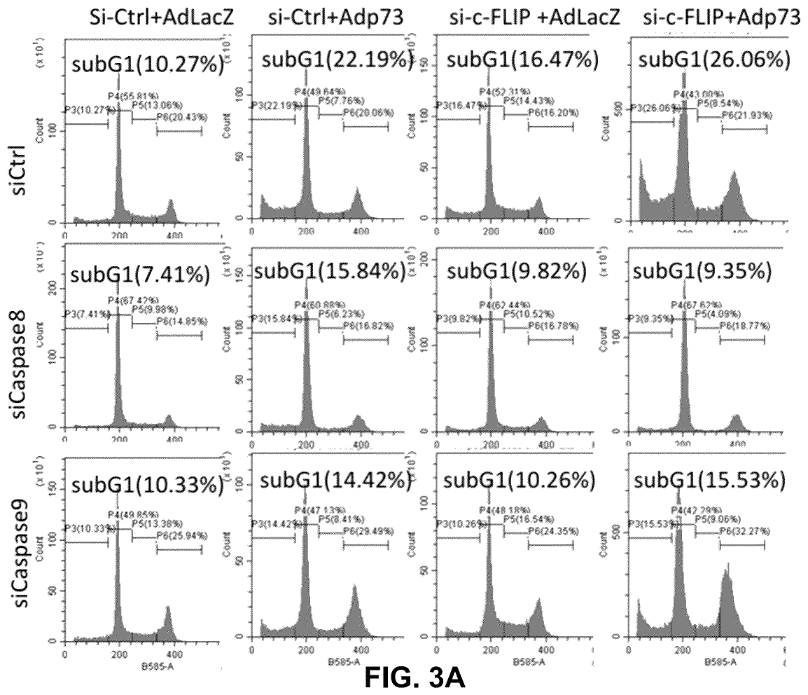

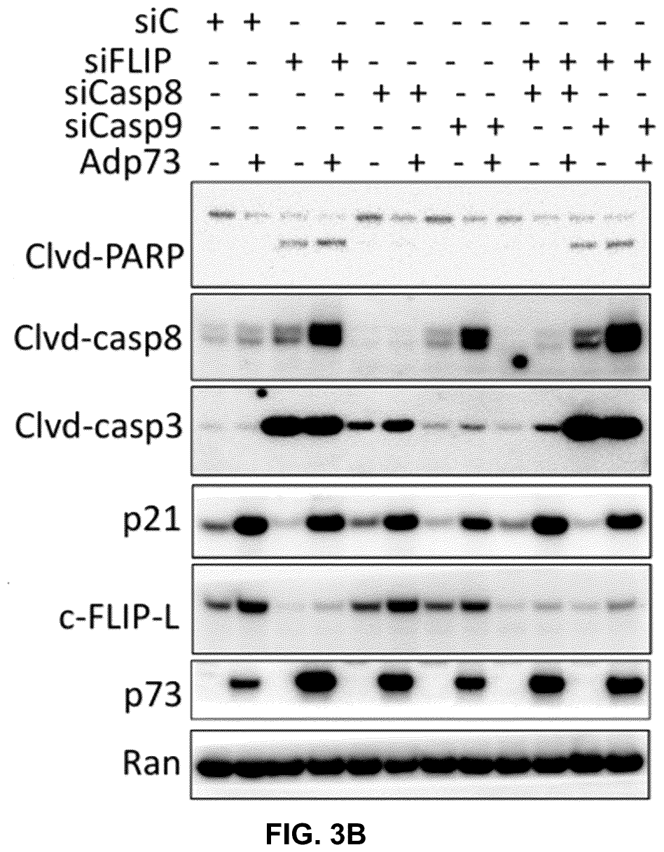

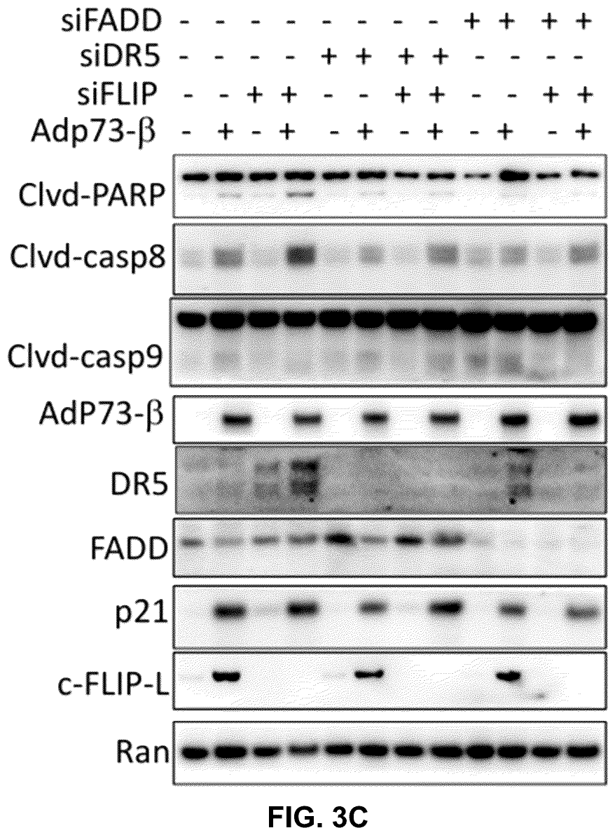

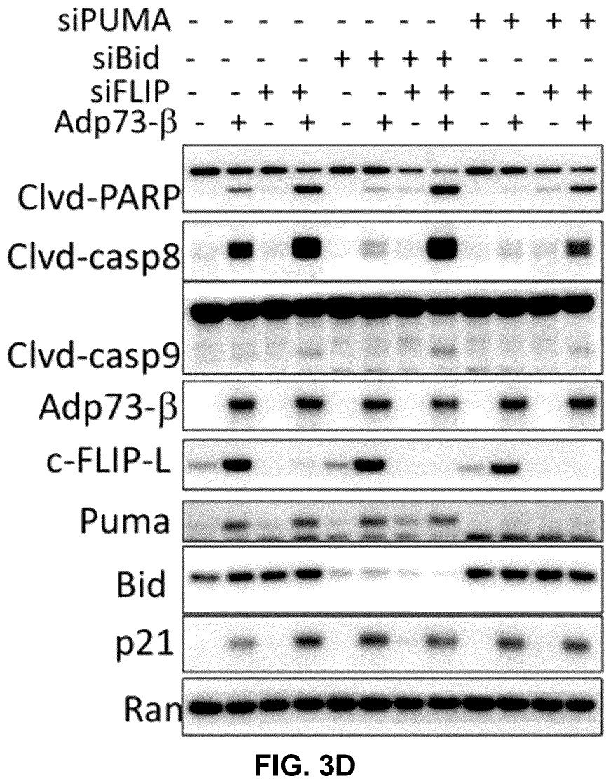

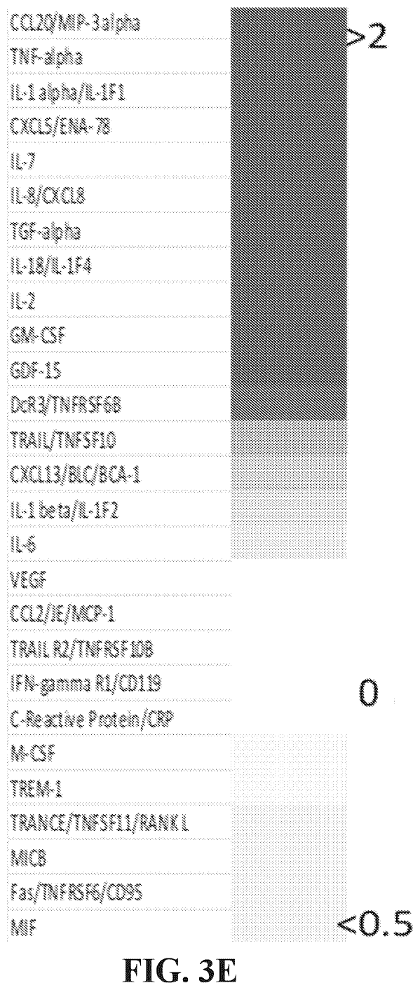

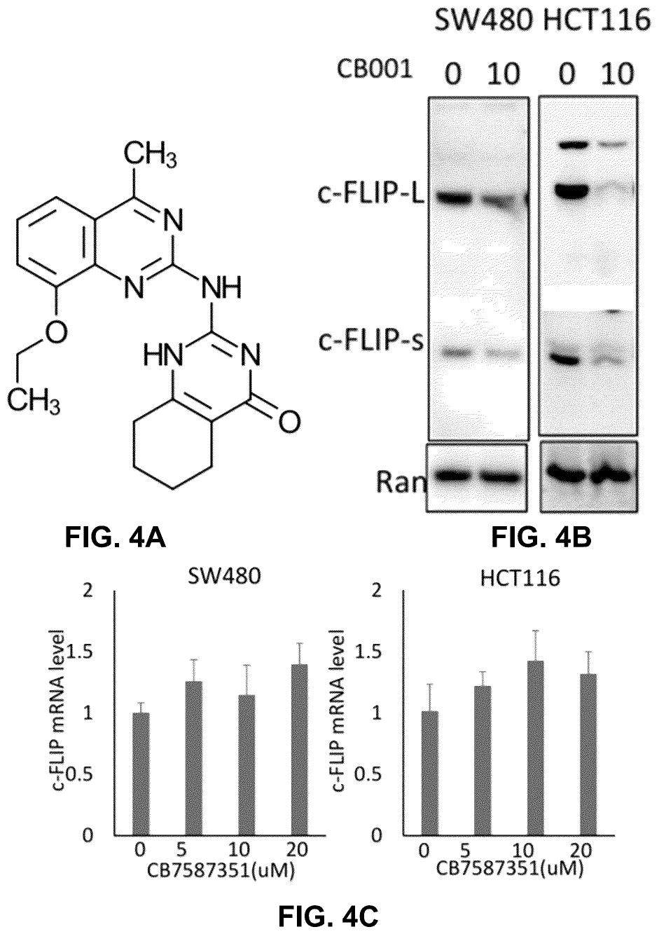

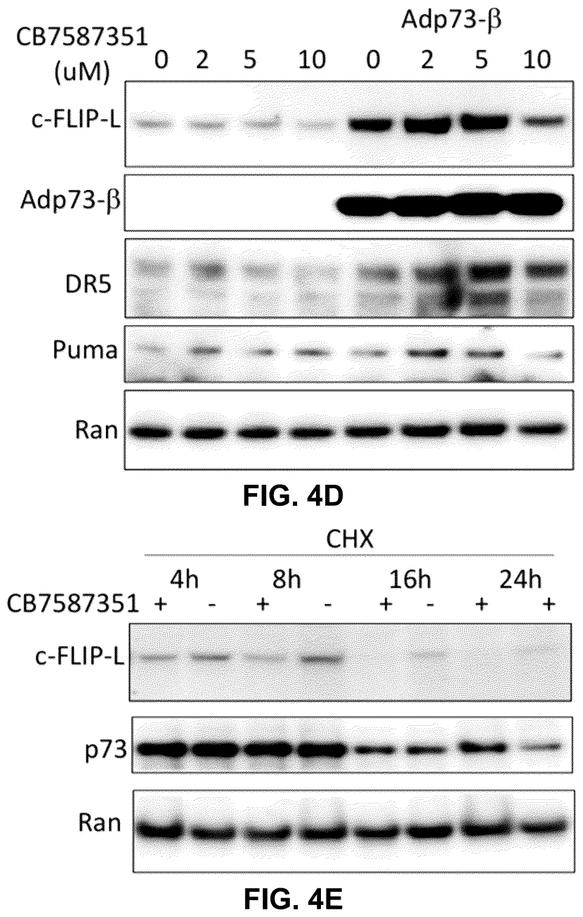

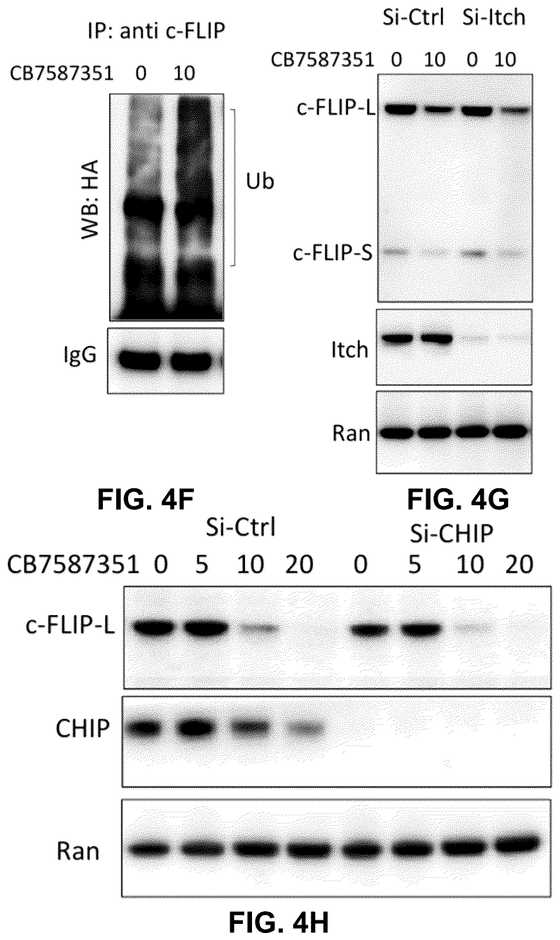

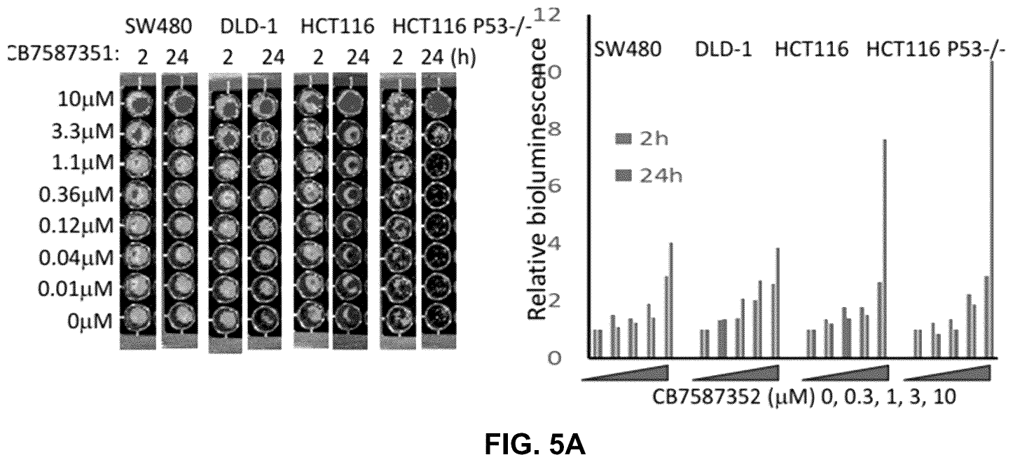

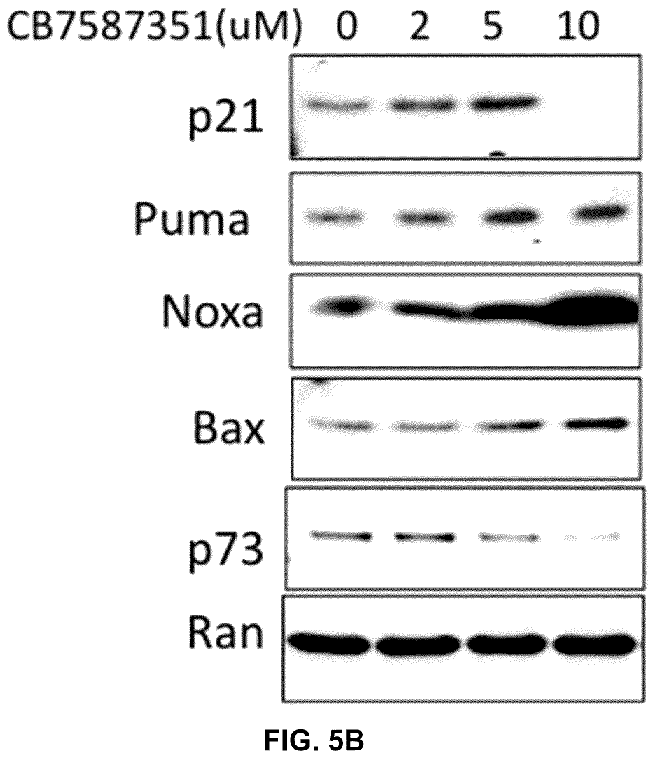

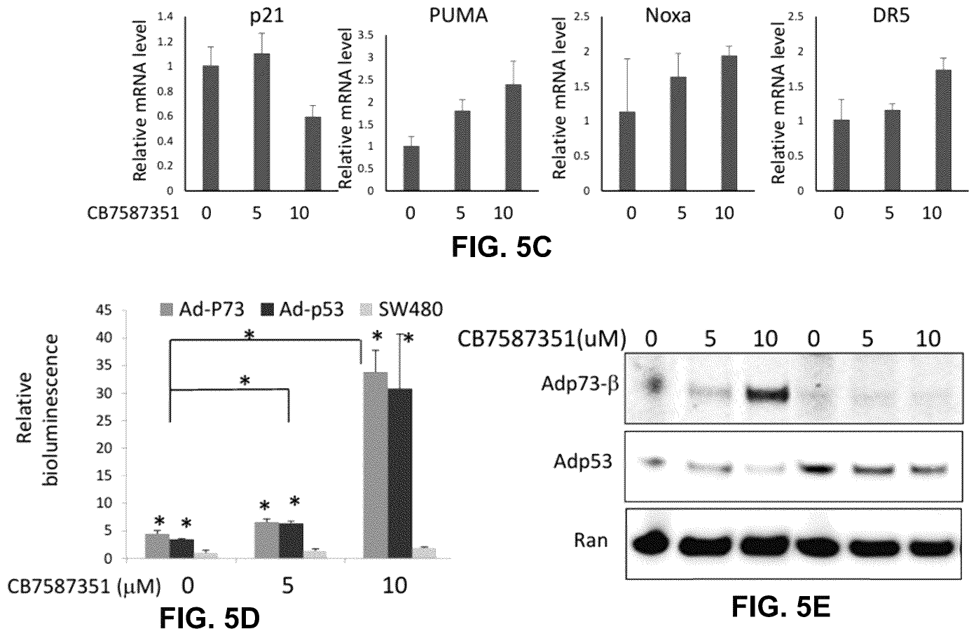

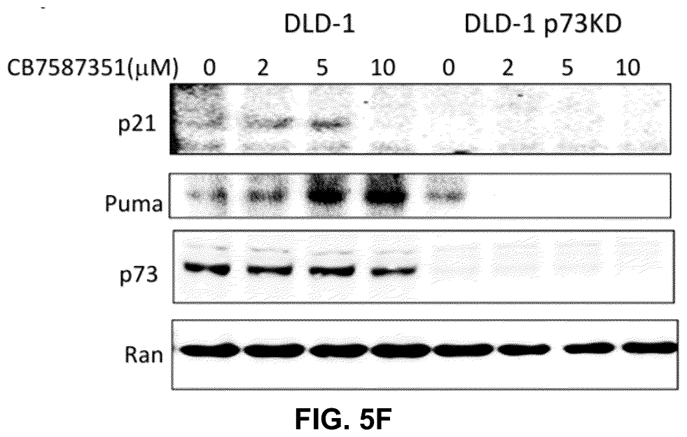

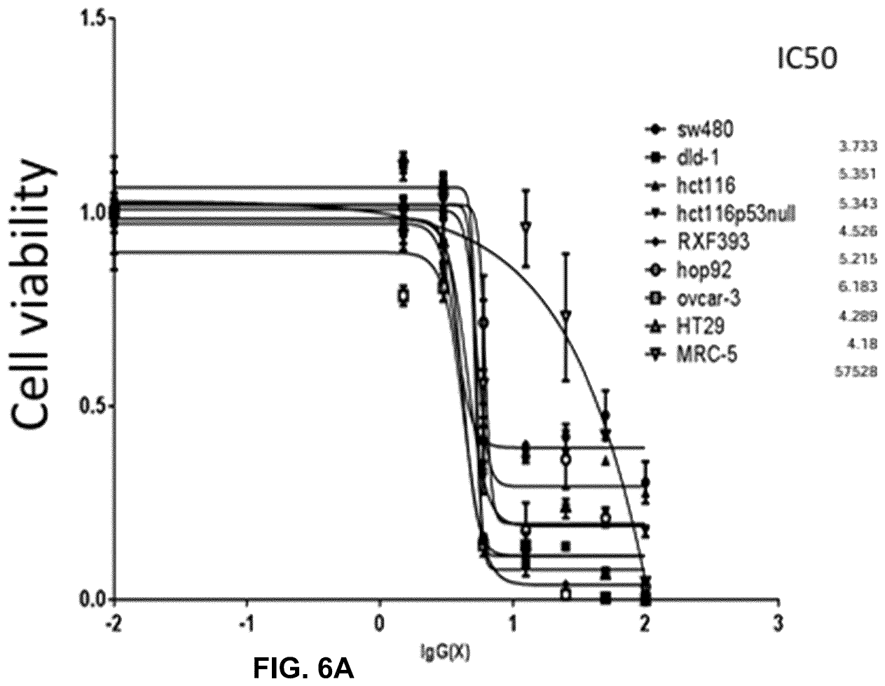

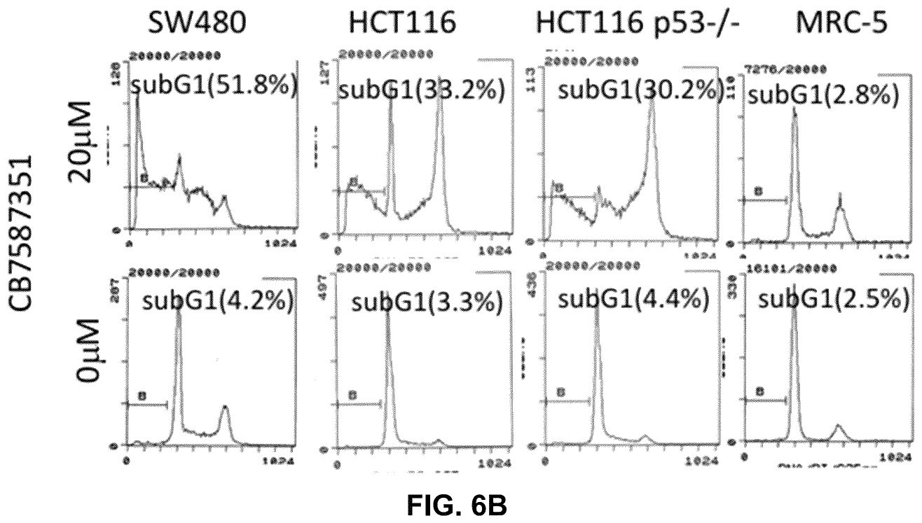

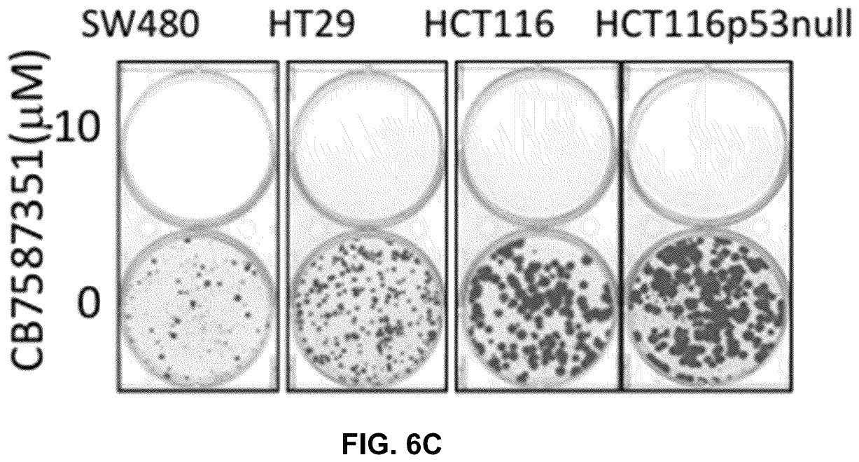

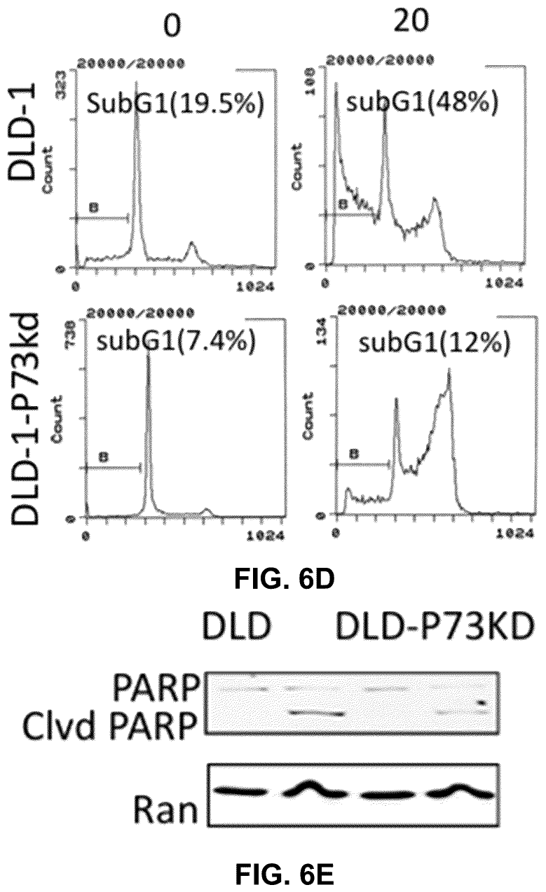

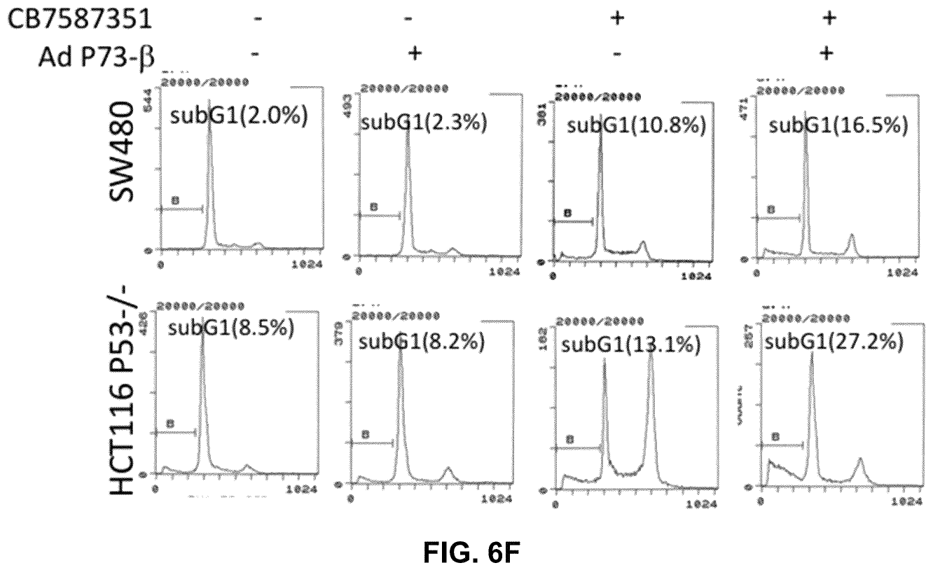

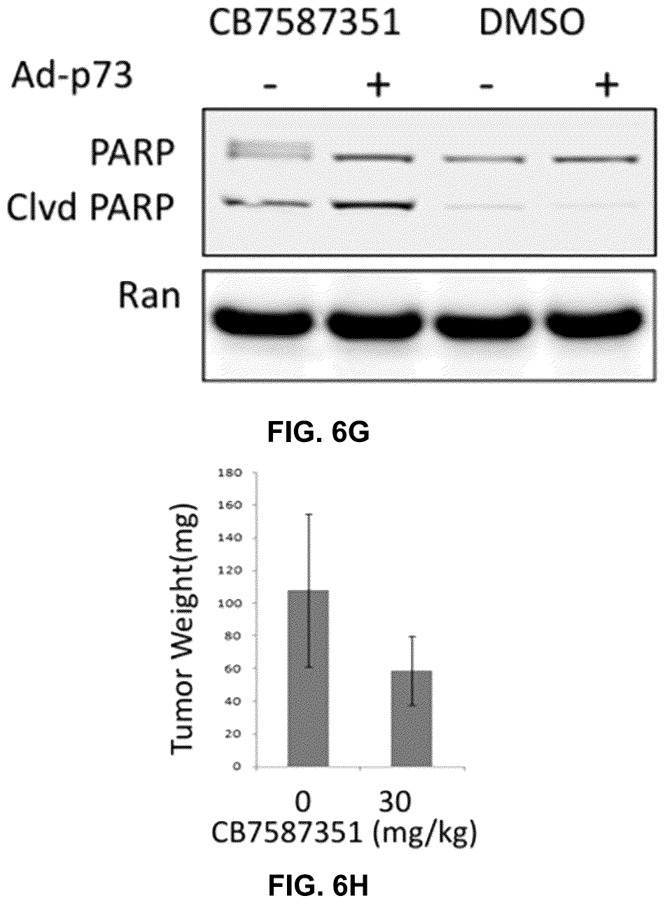

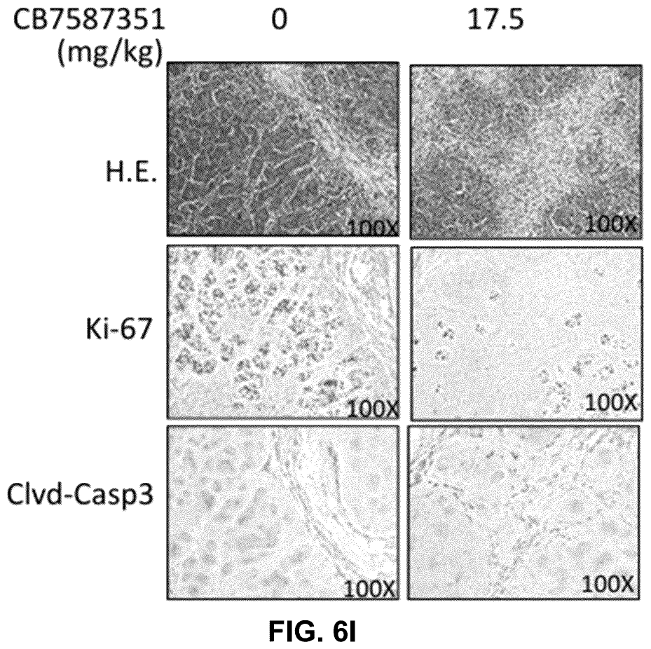

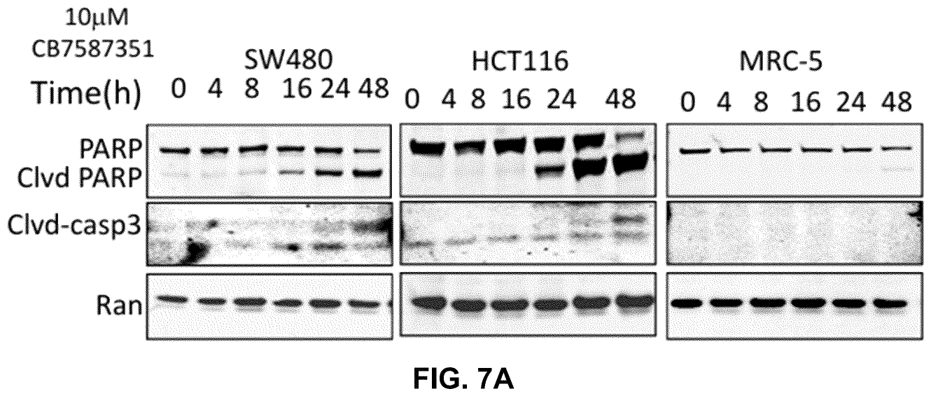

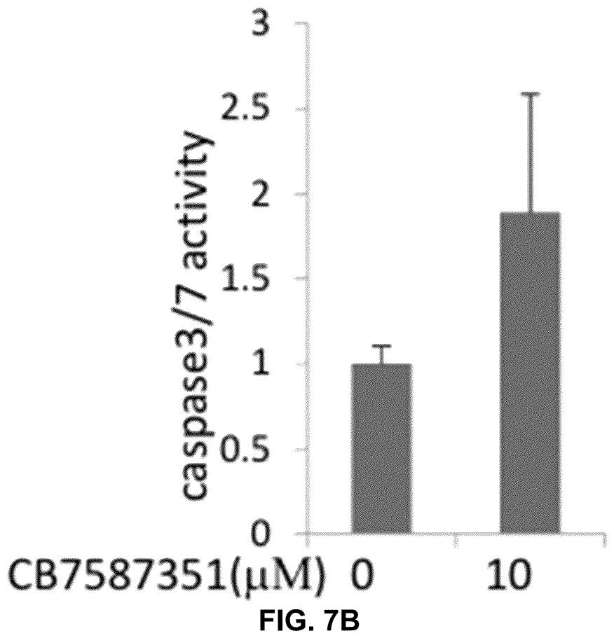

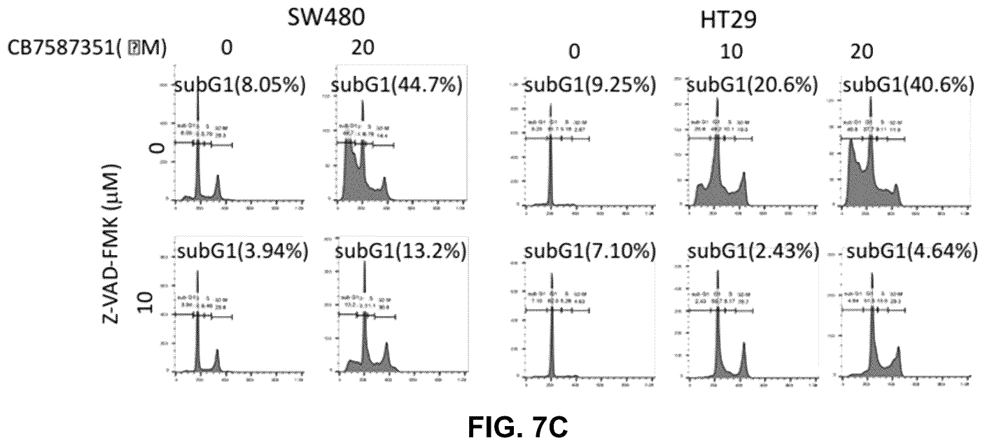

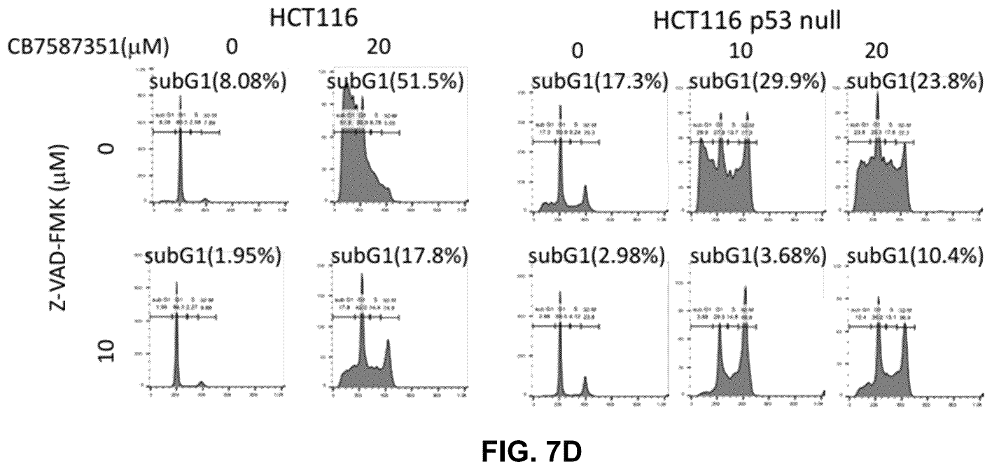

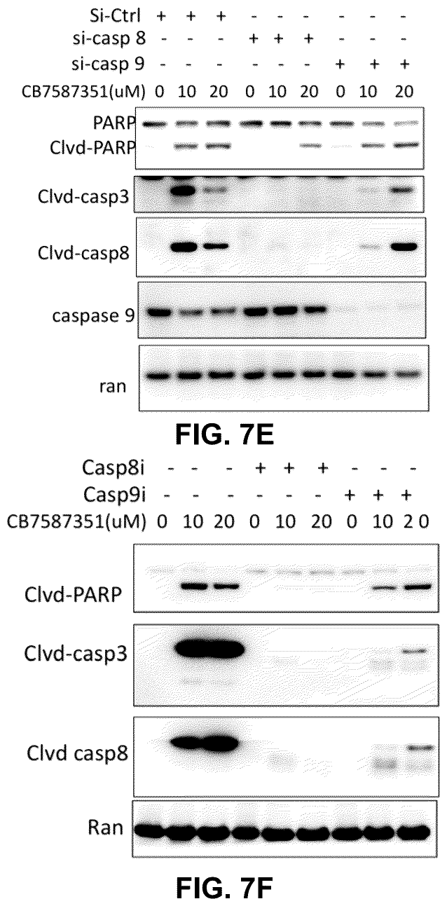

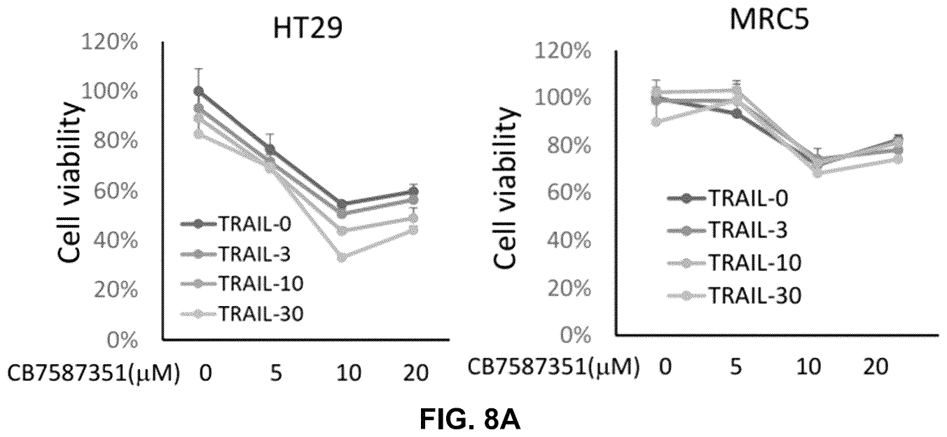

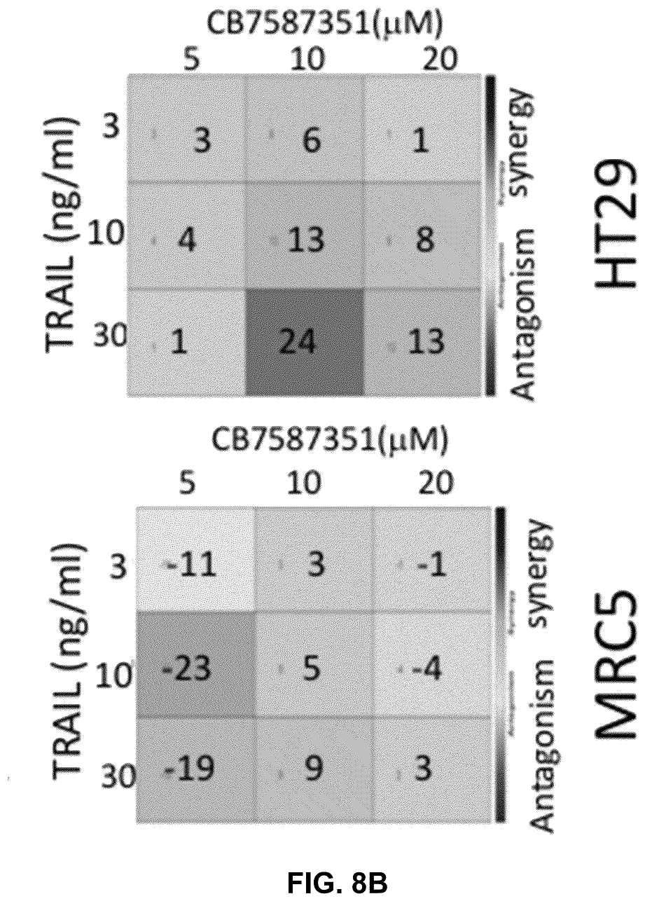

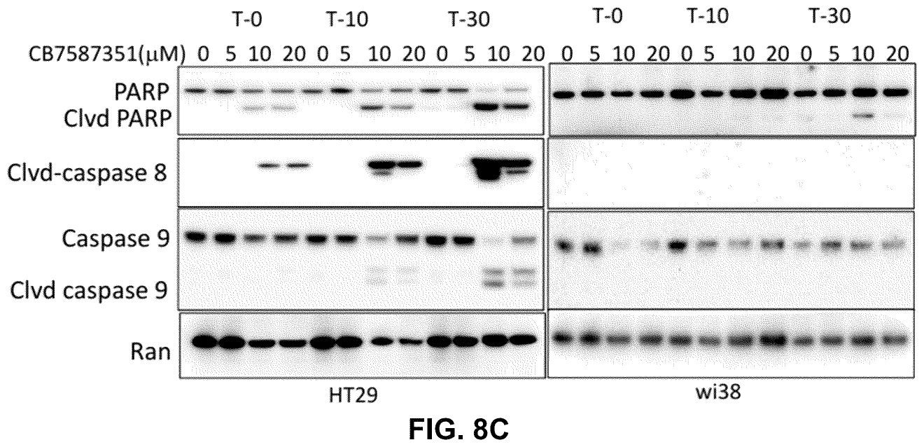

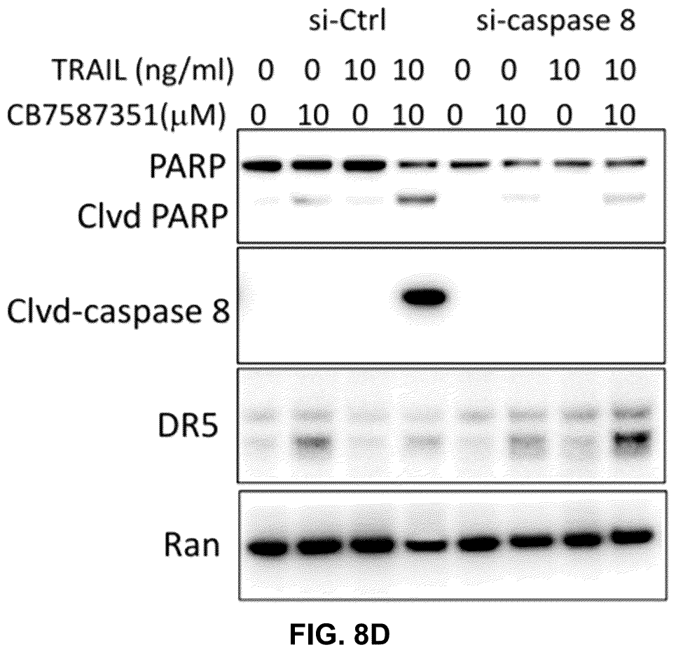

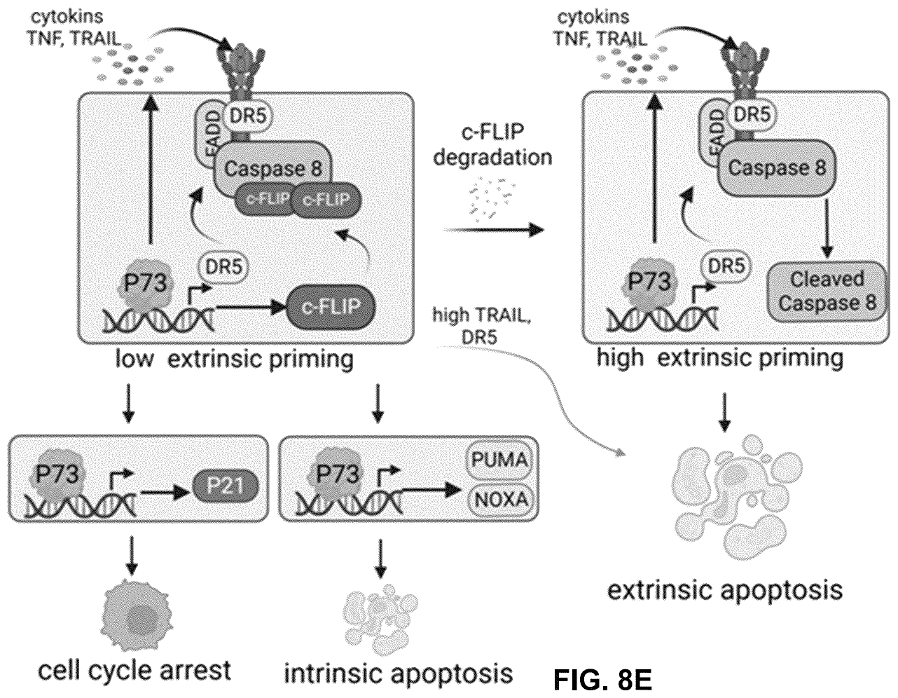

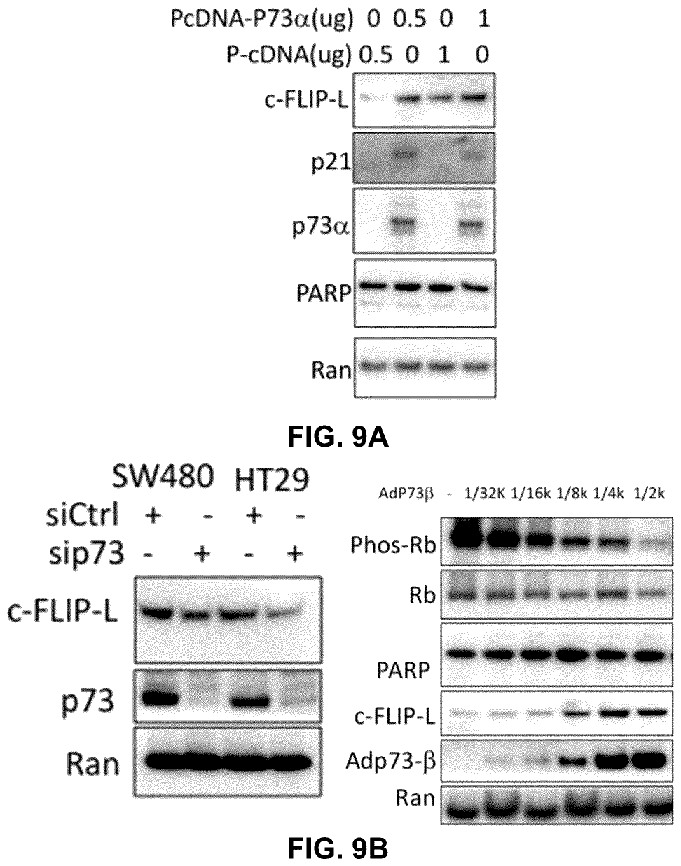

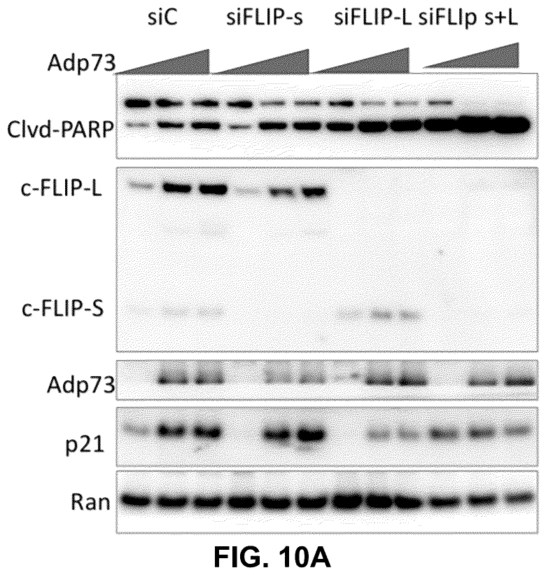

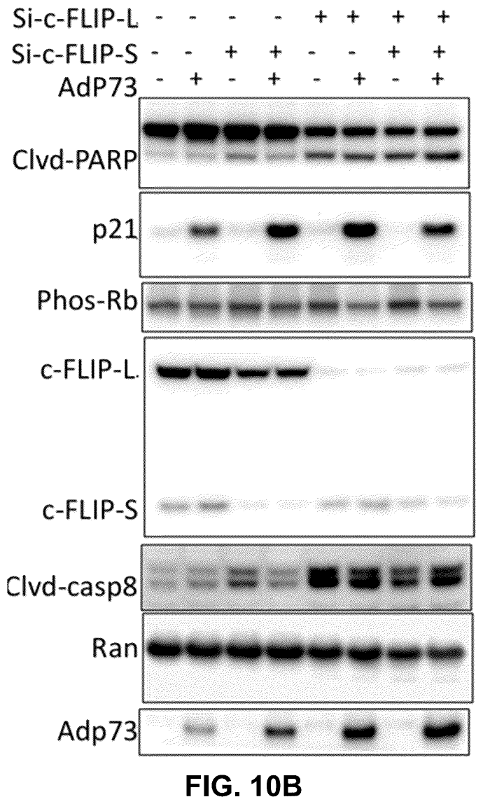

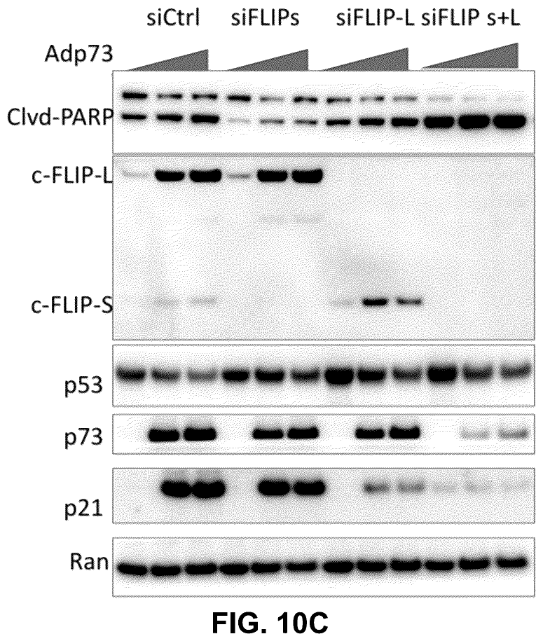

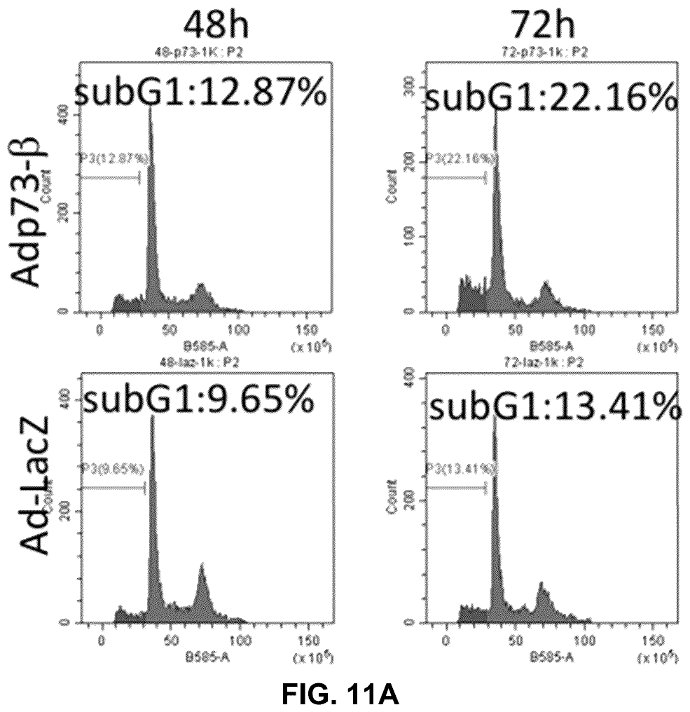

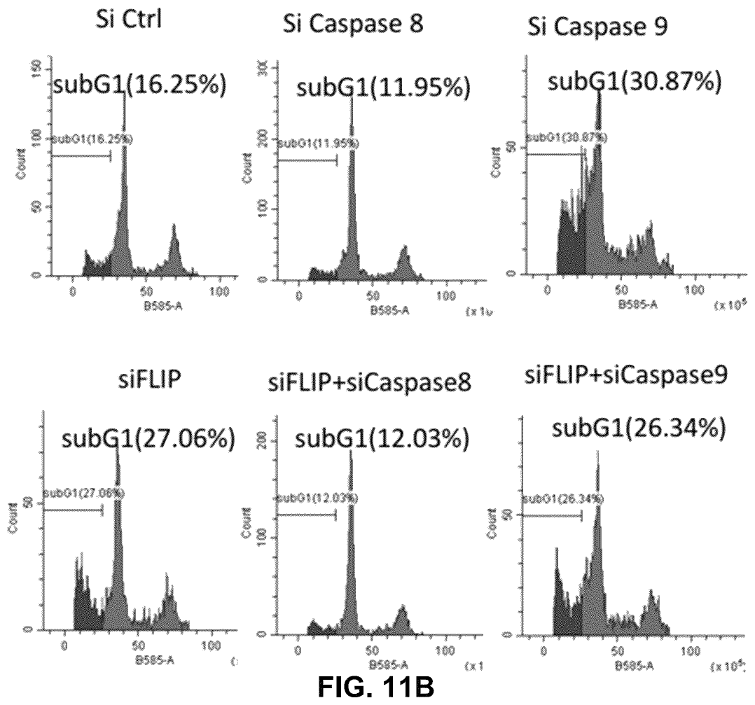

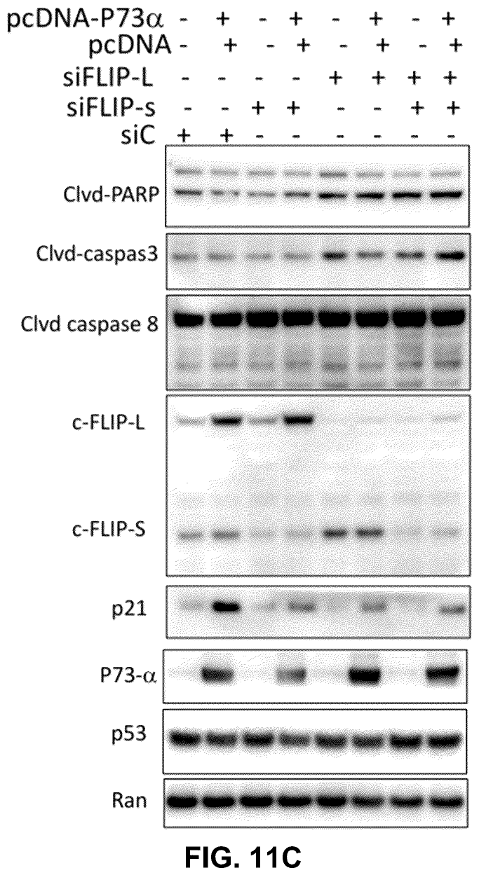

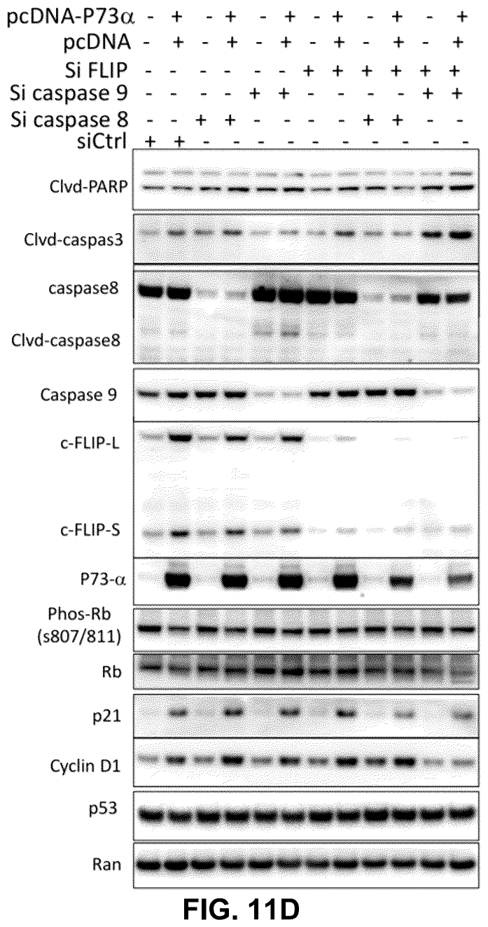

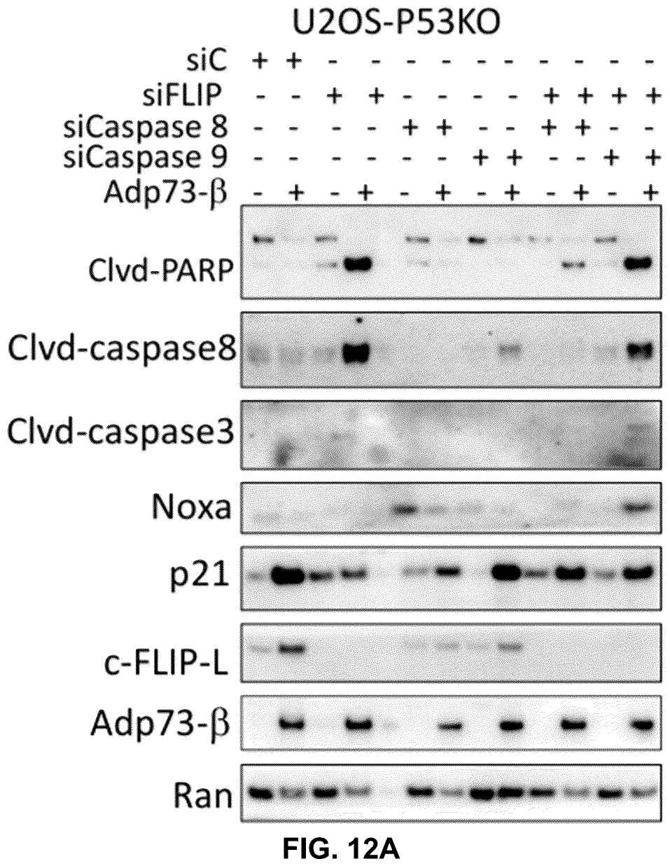

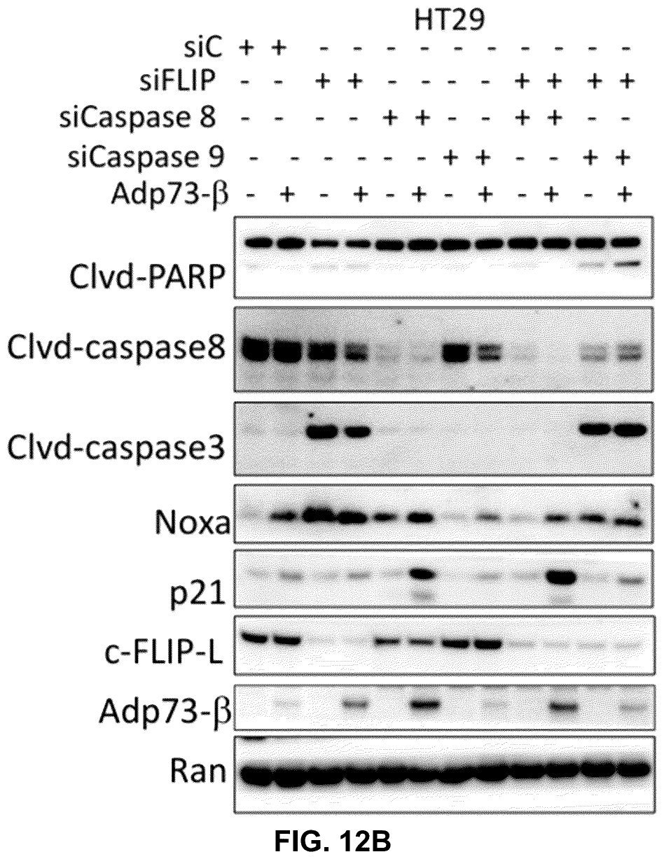

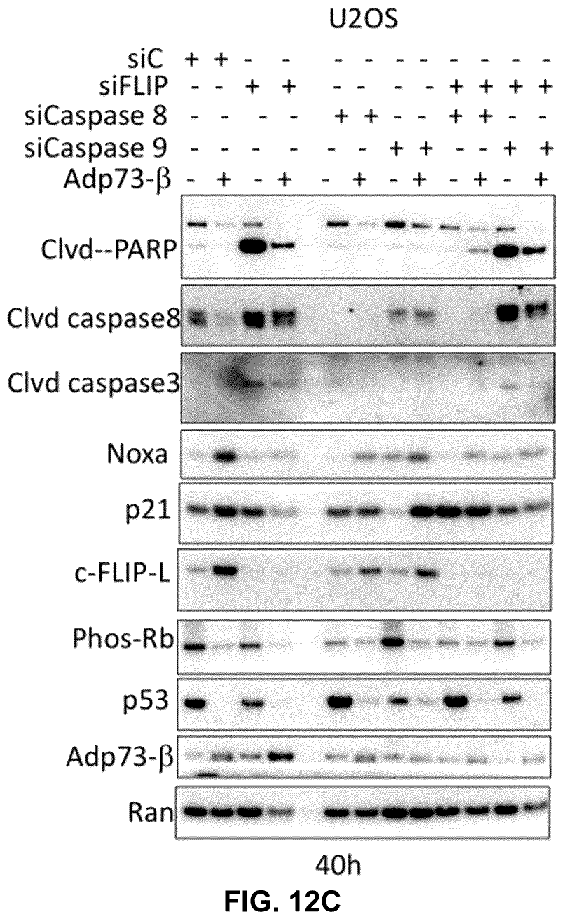

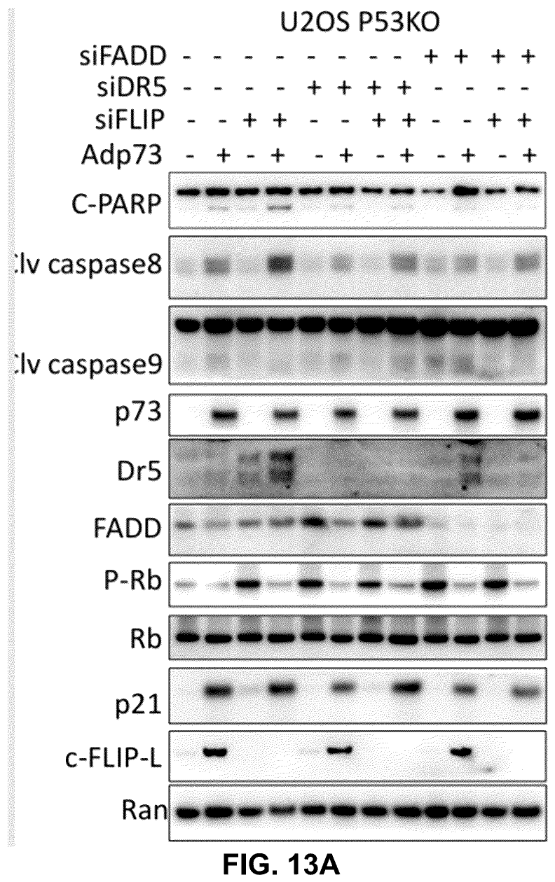

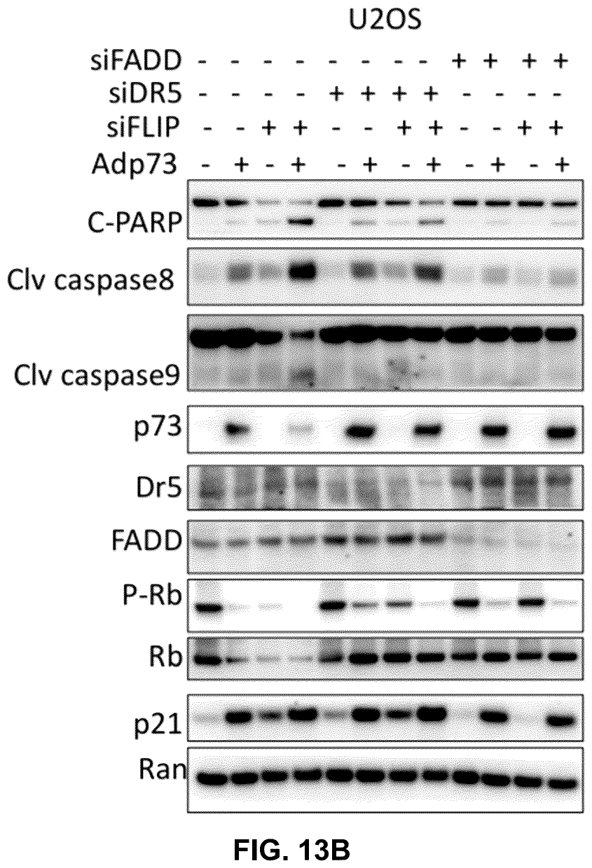

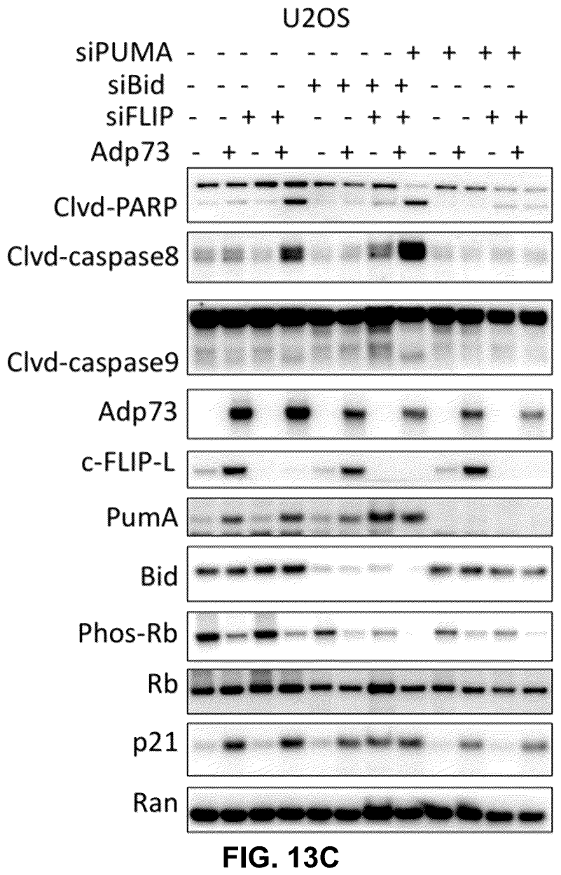

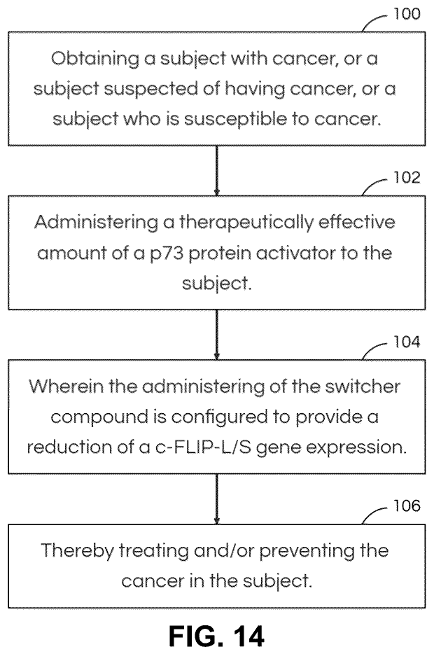

FIG. 14 is a flowchart illustrating a method in step 100 for treating cancer in a subject, according to an embodiment. At step 100, a subject with cancer, or a subject suspected of having cancer, or a subject who is susceptible to cancer may be obtained. The process may involve identifying individuals who are either diagnosed with cancer, suspected of having cancer, or at risk of developing cancer. This step may serve as the initial phase in the treatment method, setting the stage for subsequent therapeutic interventions. The identification of the subject may be based on medical history, diagnostic tests, or genetic predisposition, which may help in determining the appropriate course of action for treatment. The focus of this step may be on ensuring that the subject is accurately identified to facilitate the effective administration of the treatment protocol. The method may then proceed to the administration of a therapeutically effective amount of a p73 protein activator to the subject. The p73 protein activator may be a switcher compound, which may be configured to provide a reduction of a c-FLIP-L/S gene expression. This reduction may be operative to sensitize cancer cells to a p73-induced apoptosis in the cancer cells. The administration of the switcher compound may be aimed at enhancing the activity of the p73 protein, which may play a role in inducing apoptosis in cancer cells. The reduction of c-FLIP-L/S gene expression may be a step in sensitizing the cancer cells to undergo programmed cell death, thereby potentially inhibiting the progression of cancer. The method may ultimately aim to treat and/or prevent cancer in the subject by leveraging the therapeutic potential of the p73 protein activator. The treatment strategy may be designed to target specific molecular pathways involved in cancer cell survival, thereby offering a targeted approach to cancer therapy. The overall goal of the method may be to provide an effective treatment option for subjects with cancer, potentially improving clinical outcomes and enhancing the quality of life for patients.

In the context of step 102 (FIG. 14), the process may involve the administration of a therapeutically effective amount of a p73 protein activator to a subject. This administration may be directed towards a subject who is either diagnosed with cancer, suspected of having cancer, or is susceptible to cancer. The p73 protein activator, which may be a switcher compound, is potentially configured to enhance the activity of the p73 protein. This enhancement may be achieved through the activation of p73 as either an orthosteric or allosteric activator. The primary objective of this administration may be to provide a reduction in the expression of the c-FLIP-L/S gene. This reduction is potentially operative in sensitizing cancer cells to apoptosis induced by the p73 protein. The switcher compound, by activating the p73 protein, may transcriptionally regulate the c-FLIP gene, thereby influencing its expression. The modulation of the p73 protein may also be considered, either in addition to or instead of its activation, to alter its activity. The administration of the switcher compound may be configured to compensate for the loss of function of the p53 protein, which is related to the p73 protein, by inducing cell cycle arrest, apoptosis, or differentiation. This compensation may prevent uncontrolled cancer cell growth and tumor development. The switcher compound may also be effective in restoring the wild-type function to a mutant p53 in cancer cells. The method may include the use of specific small molecules, such as CB-7587351, RETRA, Prodigiosin, or NSC59984, which may serve as the switcher compounds. These small molecules may be provided in the form of a salt, hydrate, or solvate. The overall aim of this step is to treat and/or prevent cancer in the subject by leveraging the therapeutic potential of the p73 protein activator.

In the context of step 104 (FIG. 14), the process may involve the administration of a switcher compound, which is potentially configured to provide a reduction in the expression of the c-FLIP-L/S gene. This reduction may be operative in sensitizing cancer cells to apoptosis induced by the p73 protein. The switcher compound, as a p73 protein activator, may function as an orthosteric or allosteric activator, enhancing the activity of the p73 protein. This activation may lead to the transcriptional regulation of the c-FLIP gene, thereby modulating its expression. The reduction in c-FLIP-L/S gene expression may play a role in sensitizing cancer cells to apoptosis, which is induced by the activated p73 protein. This sensitization may be a step in the therapeutic strategy to treat cancer, as it may enhance the susceptibility of cancer cells to programmed cell death. The switcher compound, by activating the p73 protein, may compensate for the loss of function of the p53 protein, which is often inactive in cancer cells. This compensation may prevent uncontrolled cancer cell growth and tumor development by inducing cell cycle arrest, apoptosis, or differentiation. The method may also involve the use of specific small molecules, such as CB-7587351, RETRA, Prodigiosin, or NSC59984, which may serve as effective switcher compounds in this therapeutic approach. These small molecules may be administered in a therapeutically effective amount to achieve the desired reduction in c-FLIP-L/S gene expression, thereby enhancing the sensitivity of cancer cells to p73-induced apoptosis. The overall process may be aimed at treating and/or preventing cancer in the subject by leveraging the activation and modulation of the p73 protein through the administration of the switcher compound.

In the context of step 106 (FIG. 14), the process may involve the treatment and/or prevention of cancer in a subject. This step may be understood as the culmination of a series of actions that potentially lead to the therapeutic outcome. Initially, a subject with cancer, or one suspected of having cancer, may be identified. Subsequently, a therapeutically effective amount of a p73 protein activator, which may be a switcher compound, could be administered to the subject. This administration may be configured to reduce the expression of the c-FLIP-L/S gene, which may sensitize cancer cells to apoptosis induced by p73. The reduction in c-FLIP-L/S gene expression may play a role in sensitizing the cancer cells, thereby facilitating the apoptotic process. The switcher compound may act as an orthosteric or allosteric activator of the p73 protein, enhancing its activity. This activation may compensate for the loss of function of the p53 protein, which is often inactive in cancer cells, thereby preventing uncontrolled cancer cell growth and tumor development. The switcher compound may also restore wild-type function to a mutant p53, further contributing to the restoration of normal cellular functions. The overall process may involve the modulation of p73 activity, which could alter its function to achieve the desired therapeutic effect. The method may also include the identification of specific small molecules, such as CB-7587351, RETRA, Prodigiosin, or NSC59984, which may serve as effective switcher compounds. These compounds may be selected based on their ability to activate p73 and reduce c-FLIP-L/S gene expression, thereby enhancing the sensitivity of cancer cells to apoptosis. The treatment method may be tailored to the specific needs of the subject, taking into account the molecular characteristics of the cancer cells and the expression levels of relevant proteins. Through this comprehensive approach, the method may offer a potential strategy for treating and/or preventing cancer in subjects in need thereof.

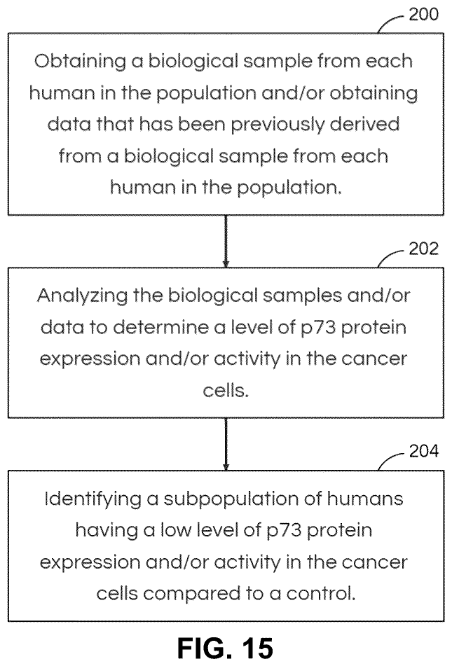

FIG. 15 is a flowchart illustrating a method for identifying a population of humans wherein an activation and/or a modulation of a p73 protein by administering a small-molecule switcher compound may provide a reduction of a c-FLIP-L/S gene expression, which is operative to sensitize any cancer cells in the human population to a p73-induced apoptosis in the cancer cells, according to an embodiment. At step 200 (FIG. 15), a biological sample may be obtained from each human in the population, or data that has been previously derived from a biological sample from each human in the population may be obtained. This step may involve the collection of relevant biological material or data that can be used for further analysis. The biological samples or data may then be analyzed to determine a level of p73 protein expression and/or activity in the cancer cells. This analysis may help in understanding the current state of p73 protein activity within the cancer cells, which is crucial for identifying potential candidates for treatment. Following this, a subpopulation of humans having a low level of p73 protein expression and/or activity in the cancer cells compared to a control may be identified. This identification process may involve comparing the analyzed data against a control to determine which individuals may benefit from the treatment. The identified subpopulation of humans may then be considered as the population wherein an activation and/or a modulation of the p73 protein by the administering of the switcher compound may provide the reduction of the c-FLIP-L/S gene expression. This reduction may be operative to sensitize the cancer cells to the p73-induced apoptosis, thereby potentially enhancing the effectiveness of the treatment. The method may thus provide a systematic approach to identifying suitable candidates for a targeted cancer treatment strategy.

In the context of analyzing biological samples and data to determine the level of p73 protein expression and activity in cancer cells, the process may involve several intricate actions and interactions. Initially, the biological samples, which may include tissue or fluid samples from the subject, can be subjected to various analytical techniques to assess the expression levels of the p73 protein. This analysis may be significant in understanding the current state of the cancer cells and their potential responsiveness to treatment. The p73 protein, known for its role in inducing apoptosis, may be evaluated for its expression levels, which can provide insights into the cancer cells' susceptibility to therapeutic interventions.

The analysis may involve the use of specific assays or molecular techniques to quantify the p73 protein expression. These techniques can include immunohistochemistry, Western blotting, or quantitative PCR, each offering a different approach to measure protein levels. The data obtained from these analyses may then be compared to control samples to determine any deviations in p73 expression, which could indicate a potential therapeutic target.

Furthermore, the activity of the p73 protein may also be assessed, which involves understanding how effectively the protein can induce apoptosis in the cancer cells. This assessment may require functional assays that evaluate the downstream effects of p73 activation, such as the induction of apoptosis-related genes or the reduction of anti-apoptotic factors like c-FLIP-L/S gene expression. The reduction of c-FLIP-L/S gene expression is particularly significant as it may sensitize the cancer cells to p73-induced apoptosis, thereby enhancing the therapeutic efficacy.

The overall goal of this step is to gather comprehensive data on the p73 protein's expression and activity, which can inform subsequent treatment decisions. By identifying the levels of p73 protein expression and activity, clinicians may be able to tailor therapeutic strategies that leverage the activation of p73 to induce apoptosis in cancer cells, potentially leading to improved treatment outcomes. This step, therefore, plays a role in the personalized treatment of cancer, where the molecular characteristics of the cancer cells guide the therapeutic approach.

At step 204 (FIG. 15), a subpopulation of humans may be identified based on their low level of p73 protein expression and/or activity in cancer cells compared to a control. This identification may involve analyzing biological samples or data to determine the p73 protein expression levels. The identified subpopulation may represent those individuals for whom the activation and/or modulation of the p73 protein by administering a switcher compound could potentially provide a reduction in c-FLIP-L/S gene expression. This reduction may sensitize the cancer cells to p73-induced apoptosis. The switcher compound may act as a p73 protein activator, potentially functioning as an orthosteric or allosteric activator, to enhance p73 activity. The administration of the switcher compound may be configured to provide a reduction of c-FLIP-L/S gene expression, which may sensitize cancer cells to apoptosis. The process may involve the use of small molecules such as CB-7587351, RETRA, Prodigiosin, or NSC59984, which may serve as effective switcher compounds. The method may aim to treat and/or prevent cancer by targeting the identified subpopulation, thereby potentially offering a tailored therapeutic approach.

In a discussion, study or a reading of the details, features, embodiments, aspects, FIGs., and/or examples of the technology disclosed herein, any of the features, embodiments, aspects, and/or examples herein can be optionally inter-combined (or inter-discussed) with the example details listed below, and any portion of any detail below can be inter-combined with any portion of any feature or example disclosed herein:

Detail 1: A method for treating cancer in a subject, the method comprising: administering to the subject a therapeutically effective amount of a p73 protein activator, wherein the p73 protein activator is a switcher compound configured to provide a reduction of a c-FLIP-L/S gene expression, thereby sensitizing cancer cells in the subject to a p73-induced apoptosis and treating the cancer in the subject.

Detail 2: The method of detail 1, wherein the subject has cancer, is suspected of having cancer, or is susceptible to cancer.

Detail 3: The method of detail 1, wherein the p73 protein activator is administered in a dosage form selected from the group consisting of a pill, a tablet, a capsule, a liquid, an intravenous injection, an intramuscular injection, a subcutaneous injection, an inhalant, and a suppository.

Detail 4: The method of detail 1, wherein the p73 protein activator is administered orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

Detail 5: The method of detail 1, wherein the cancer is selected from the group consisting of lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

Detail 6: The method of detail 1, wherein the switcher compound is selected from the group consisting of a small molecule, a peptide, a polypeptide, a protein, an antibody, a ribozyme, a dsRNA, an siRNA, an miRNA, an aptamer, and combinations thereof.

Detail 7: The method of detail 1, wherein the switcher compound is administered in an amount ranging from about 0.01 mg/kg to about 100 mg/kg of body weight of the subject.

Detail 8: The method of detail 1, wherein the switcher compound is administered in an amount ranging from about 0.1 mg/kg to about 50 mg/kg of body weight of the subject.

Detail 9: The method of detail 1, wherein the switcher compound is administered in an amount ranging from about 1 mg/kg to about 20 mg/kg of body weight of the subject.

Detail 10: The method of detail 1, wherein the switcher compound is administered in a single dose or in multiple doses.

Detail 11: The method of detail 1, wherein the switcher compound is administered daily, weekly, biweekly, monthly, bimonthly, quarterly, or annually.

Detail 12: The method of detail 1, wherein the switcher compound is administered for a period of time ranging from about 1 day to about 1 year.

Detail 13: The method of detail 1, wherein the switcher compound is administered for a period of time ranging from about 1 week to about 6 months.

Detail 14: The method of detail 1, wherein the switcher compound is administered for a period of time ranging from about 2 weeks to about 3 months.

Detail 15: The method of detail 1, further comprising administering to the subject at least one additional therapeutic agent.

Detail 16: The method of detail 15, wherein the at least one additional therapeutic agent is selected from the group consisting of a chemotherapeutic agent, a radiotherapeutic agent, an immunotherapeutic agent, and combinations thereof.

Detail 17: The method of detail 15, wherein the at least one additional therapeutic agent and the p73 protein activator are administered simultaneously, sequentially, or separately.

Detail 18: The method of detail 1, wherein the reduction of the c-FLIP-L/S gene expression ranges from about 10% to about 100% compared to a control level.

Detail 19: The method of detail 1, wherein the reduction of the c-FLIP-L/S gene expression ranges from about 20% to about 80% compared to a control level.

Detail 20: The method of detail 1, wherein the reduction of the c-FLIP-L/S gene expression ranges from about 30% to about 70% compared to a control level.

Detail 21: A method for treating cancer in a subject, the method comprising: administering to the subject a therapeutically effective amount of a p73 protein activator, wherein the p73 protein activator is a switcher compound configured to provide a reduction of a c-FLIP-L/S gene expression, thereby sensitizing cancer cells in the subject to a p73-induced apoptosis and treating the cancer in the subject, and wherein the switcher compound is selected from the group consisting of a small molecule modulator of p73 protein, a small molecule activator of p73 protein, and combinations thereof.

Detail 22: The method of detail 21, wherein the subject has cancer, is suspected of having cancer, or is susceptible to cancer, and wherein the cancer is selected from the group consisting of lung cancer, bone cancer, pancreatic cancer, skin cancer, cancer of the head or neck, cutaneous or intraocular malignant melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

Detail 23: The method of detail 21, wherein the p73 protein activator is administered in a dosage form selected from the group consisting of a pill, a tablet, a capsule, a liquid, an intravenous injection, an intramuscular injection, a subcutaneous injection, an inhalant, and a suppository, and wherein the p73 protein activator is administered orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, or intrathecally.

Detail 24: The method of detail 21, wherein the switcher compound is a small molecule activator of the p73 protein comprising: (2R,4S)-2-butyl-6-chloro-4-(2-chlorophenyl)-1,2,3,4-tetrahydroquinoline; (2S,4R)-4-(4-chlorophenyl)-2-ethyl-6-fluoro-1,2,3,4-tetrahydroquinoline; (2R,4S)-2-benzyl-6-bromo-4-(3-bromophenyl)-1,2,3,4-tetrahydroquinoline; (2S,4R)-2-butyl-4-(2,4-dichlorophenyl)-6-iodo-1,2,3,4-tetrahydroquinoline; (2R,4S)-4-(3-chloro-4-fluorophenyl)-2-hexyl-6-methyl-1,2,3,4-tetrahydroquinoline; small molecule CB-7587351; small molecule RETRA; small molecule Prodigiosin; small molecule NSC59984; or a combination thereof; and/or a salt, hydrate and/or a solvate thereof.

Detail 25: The method of detail 21, wherein the switcher compound is administered in an amount ranging from about 0.01 mg/kg to about 100 mg/kg of body weight of the subject, preferably from about 0.1 mg/kg to about 50 mg/kg of body weight of the subject, and more preferably from about 1 mg/kg to about 20 mg/kg of body weight of the subject.

Detail 26: The method of detail 21, wherein the switcher compound is administered in a single dose or in multiple doses, and wherein the switcher compound is administered daily, weekly, biweekly, monthly, bimonthly, quarterly, or annually.

Detail 27: The method of detail 21, wherein the switcher compound is administered for a period of time ranging from about 1 day to about 1 year, preferably from about 1 week to about 6 months, and more preferably from about 2 weeks to about 3 months.

Detail 28: The method of detail 21, further comprising administering to the subject at least one additional therapeutic agent selected from the group consisting of a chemotherapeutic agent, a radiotherapeutic agent, an immunotherapeutic agent, and combinations thereof, wherein the at least one additional therapeutic agent and the p73 protein activator are administered simultaneously, sequentially, or separately.

Detail 29: The method of detail 21, wherein the reduction of the c-FLIP-L/S gene expression ranges from about 10% to about 100% compared to a control level, preferably from about 20% to about 80% compared to a control level, and more preferably from about 30% to about 70% compared to a control level.

Detail 30: The method of detail 24, wherein the small molecule activator of p73 protein is selected from the group consisting of 2-amino-5-bromopyridine, 2-amino-5-chloropyridine, 2-amino-5-fluoropyridine, 2-amino-5-iodopyridine, 2-amino-5-methylpyridine, and combinations thereof.

Detail 31: The method of detail 21, wherein the p73 protein activator enhances the transcriptional activity of p73 protein by at least 1.5-fold, preferably by at least 2-fold, and more preferably by at least 3-fold compared to a control level.

Detail 32: The method of detail 21, wherein the p73 protein activator increases the stability of p73 protein by reducing its ubiquitination and proteasomal degradation, thereby increasing the half-life of p73 protein by at least 30%, preferably by at least 50%, and more preferably by at least 100% compared to a control level.

Detail 33: The method of detail 21, wherein the p73 protein activator disrupts the interaction between p73 protein and its negative regulators, such as MDM2, MDMX, and Itch, thereby preventing the inhibition of p73 transcriptional activity and increasing its pro-apoptotic function in cancer cells.

Detail 34: The method of detail 21, wherein the p73 protein activator enhances the acetylation of p73 protein by histone acetyltransferases, such as p300 and CBP, thereby increasing the DNA-binding affinity and transcriptional activity of p73 protein.

Detail 35: The method of detail 21, wherein the p73 protein activator inhibits the deacetylation of p73 protein by histone deacetylases, such as HDAC1 and SIRT1, thereby maintaining the acetylation level and transcriptional activity of p73 protein.

Detail 36: The method of detail 21, wherein the p73 protein activator promotes the phosphorylation of p73 protein by protein kinases, such as c-Abl, p38 MAPK, and JNK, thereby enhancing the stability and pro-apoptotic function of p73 protein.

Detail 37: The method of detail 21, wherein the p73 protein activator inhibits the dephosphorylation of p73 protein by protein phosphatases, such as PP2A and Wip1, thereby maintaining the phosphorylation level and pro-apoptotic activity of p73 protein.

Detail 38: The method of detail 21, wherein the p73 protein activator stimulates the expression of p73 target genes involved in apoptosis, such as Bax, Puma, Noxa, and p53AIP1, thereby promoting the apoptotic death of cancer cells.

Detail 39: The method of detail 21, wherein the p73 protein activator inhibits the expression of anti-apoptotic genes, such as Bcl-2, Bcl-xL, and survivin, thereby sensitizing cancer cells to apoptosis induced by chemotherapeutic agents and radiotherapy.

Detail 40: The method of detail 21, wherein the p73 protein activator synergizes with other anti-cancer agents, such as cisplatin, doxorubicin, and etoposide, to enhance the therapeutic efficacy and overcome drug resistance in cancer treatment.

Detail 41: The method of detail 21, wherein the small molecule modulator of p73 protein comprises: 2-amino-4,6-dimethylpyridine; 2-amino-4-methylpyridine; 2-amino-6-methylpyridine; 2-aminopyridine; 4-aminopyridine; 2-amino-3-bromopyridine; 2-amino-3-chloropyridine; 2-amino-3-fluoropyridine; 2-amino-3-iodopyridine; 2-amino-3-methylpyridine; 2-amino-4-bromopyridine; 2-amino-4-chloropyridine; 2-amino-4-fluoropyridine; 2-amino-4-iodopyridine; 2-amino-5-bromopyridine; 2-amino-5-chloropyridine; 2-amino-5-fluoropyridine; 2-amino-5-iodopyridine; 2-amino-5-methylpyridine; 2-amino-6-bromopyridine; 2-amino-6-chloropyridine; 2-amino-6-fluoropyridine; 2-amino-6-iodopyridine; and/or a combination thereof.

Detail 42: A method for treating cancer in a subject in need thereof, the method comprising the steps of: (1) obtaining a subject with cancer, or a subject suspected of having cancer, or a subject who is susceptible to cancer, wherein the cancer is selected from the group consisting of lung cancer, breast cancer, colorectal cancer, prostate cancer, pancreatic cancer, liver cancer, bladder cancer, cervical cancer, kidney cancer, melanoma, leukemia, lymphoma, and brain cancer, and wherein the cancer is characterized by a p53 mutation, a p53 deletion, or an epigenetic silencing of the p53 gene; (2) administering a therapeutically effective amount of a p73 protein activator to the subject, wherein the p73 protein activator is a switcher compound, and wherein the switcher compound is administered orally, intravenously, intramuscularly, subcutaneously, intradermally, intranasally, or rectally, and wherein the switcher compound is administered in a dosage form selected from the group consisting of a tablet, a capsule, a solution, a suspension, an emulsion, a powder, a granule, a suppository, an injection, an infusion, an inhalation, a spray, a lotion, an ointment, a cream, a gel, a patch, and a sustained-release formulation; wherein the administering of the switcher compound is configured to provide a reduction of a c-FLIP-L/S gene expression which is operative to sensitize cancer cells to a p73-induced apoptosis in the cancer cells, and wherein the reduction of the c-FLIP-L/S gene expression is at least 20% compared to a control, and wherein the p73-induced apoptosis results in a reduction of tumor size, a reduction of tumor growth rate, a reduction of metastasis, an increase in overall survival, an increase in progression-free survival, or an amelioration of one or more symptoms of the cancer; thereby treating and/or preventing the cancer in the subject, wherein treating the cancer comprises reducing tumor size, reducing tumor growth rate, reducing metastasis, increasing overall survival, increasing progression-free survival, or ameliorating one or more symptoms of the cancer.

Detail 43: The method of detail 42, wherein the p73 activating switcher compound is administered in an amount effective to provide a reduction of c-FLIP-L/S gene expression operative to sensitize the cancer cells to p73-induced apoptosis, and wherein the amount is from about 0.01 mg/kg to about 100 mg/kg body weight of the subject, and wherein the switcher compound is administered once daily, twice daily, thrice daily, once every other day, once weekly, once every two weeks, or once monthly.

Detail 44: The method of detail 42, wherein a related p53 tumor suppressor protein is inactive in the cancer cells due to a mutation, deletion, or epigenetic silencing of the p53 gene, and wherein the mutation is a missense mutation, a nonsense mutation, a frameshift mutation, or a splice site mutation.

Detail 45: The method of detail 42, where the administration of the switcher compound is configured to compensate for a loss of a protein p53′s function by inducing cell cycle arrest, apoptosis or programmed cell death, and/or differentiation, thereby preventing an uncontrolled cancer cell growth and tumor development, and wherein the switcher compound induces cell cycle arrest at the G1/S checkpoint or the G2/M checkpoint by upregulating p21, p27, GADD45, or 14-3-3σ.

Detail 46: The method of detail 42, wherein the switcher compound activates p73 which transcriptionally regulates the c-FLIP gene and/or an expression of the c-FLIP gene by binding to a p73 response element in the c-FLIP gene promoter, and wherein the p73 response element comprises the sequence RRRCWWGYYY (SEQ ID NO: 7), wherein R is a purine, W is A or T, and Y is a pyrimidine.

Detail 47: The method of detail 42, wherein the switcher compound activates p73 as an orthosteric activator by directly binding to the p73 protein at the DNA binding domain and/or as an allosteric activator by binding to a site distinct from the p73 active site and enhancing the activity of the p73 protein by inducing a conformational change.

Detail 48: The method of detail 42, wherein the switcher compound is effective to restore wild-type function to a mutant p53 in one or more cancer cells in the subject by altering the conformation of the mutant p53 protein to a wild-type conformation, and wherein the mutant p53 has a mutation in the DNA binding domain.

Detail 49: The method of detail 42, further comprising the switcher compound is a modulator of the p73 either in addition to being an activator and/or instead of being an activator, wherein the modulator enhances the stability, half-life, or binding affinity of the p73 protein by inhibiting MDM2-mediated degradation of p73 or by promoting the acetylation of p73.

Detail 50: The method of detail 42, wherein the switcher compound comprises small molecule CB-7587351, small molecule RETRA, small molecule Prodigiosin, small molecule NSC59984, or a combination thereof; and/or a pharmaceutically acceptable salt, hydrate and/or a solvate thereof, and wherein the switcher compound has a binding affinity for p73 with a dissociation constant (Kd) of less than 1 μM.

Detail 51: The method of detail 42, wherein the switcher compound comprises one or more of: small molecule PRIMA-1 or PRIMA-1MET; small molecule MIRA-1; small molecule STIMA-1; small molecule 3-Benzoylacrylic acid; small molecule Prodigiosin; small molecule Nutlin-3; small molecule RITA; small molecule Stictic acid; small molecule CP-31398; small molecule RETRA; small molecule NSC59984; small molecule CB-7587351; and/or a pharmaceutically acceptable salt, hydrate and/or a solvate thereof, and wherein the switcher compound has an EC50 for activating p73 of less than 1 μM in a cell-based assay.

Detail 52: A pharmaceutical composition comprising a p73 protein activator, wherein the p73 protein activator is a switcher compound selected from the group consisting of: 2-[(E)-2-(3,4-dihydroxyphenyl)ethenyl]-1-benzofuran-6-ol (CB-7587351); 2,5-bis(5-hydroxymethyl-2-thienyl)furan (RETRA); 4-methoxy-5-[(Z)-2-pyridin-3-ylvinyl]-1H-pyrrole-2-carbaldehyde (Prodigiosin); 2-[(E)-2-(4-nitrophenyl)ethenyl]-1-benzofuran-5-ol (NSC59984); and/or a pharmaceutically acceptable salt, hydrate and/or a solvate thereof; wherein the composition is an effective switcher compound in a method comprising the steps of: (1) obtaining a subject with cancer, or a subject suspected of having cancer, or a subject who is susceptible to cancer, wherein the cancer is selected from the group consisting of lung cancer, breast cancer, colorectal cancer, prostate cancer, pancreatic cancer, liver cancer, bladder cancer, cervical cancer, kidney cancer, melanoma, leukemia, lymphoma, and brain cancer, and wherein the cancer is characterized by a p53 mutation, a p53 deletion, or an epigenetic silencing of the p53 gene; (2) administering a therapeutically effective amount of a p73 protein activator to the subject, wherein the p73 protein activator is a switcher compound; wherein the administering of the switcher compound is configured to provide a reduction of a c-FLIP-L/S gene expression which is operative to sensitize cancer cells to a p73-induced apoptosis in the cancer cells; thereby treating and/or preventing the cancer in the subject, and wherein the composition further comprises one or more pharmaceutically acceptable carriers, diluents, or excipients.

Detail 53: The composition of detail 52, wherein the switcher compound is a modulator of the p73 either in addition to being an activator and/or instead of being an activator, wherein the modulator enhances the stability, half-life, or binding affinity of the p73 protein by inhibiting MDM2-mediated degradation of p73 or by promoting the acetylation of p73.

Detail 54: The composition of detail 52, wherein the switcher compound is effective to restore wild-type function to a mutant p53 in one or more cancer cells by altering the conformation of the mutant p53 protein to a wild-type conformation, and wherein the mutant p53 has a mutation in the DNA binding domain.

Detail 55: The composition of detail 52, wherein the switcher compound activates p73 as an orthosteric activator by directly binding to the p73 protein at the DNA binding domain and/or as an allosteric activator by binding to a site distinct from the p73 active site and enhancing the activity of the p73 protein by inducing a conformational change.

Detail 56: The composition of detail 52, wherein the switcher compound activates p73 which transcriptionally regulates the c-FLIP gene and/or an expression of the c-FLIP gene by binding to a p73 response element in the c-FLIP gene promoter, and wherein the p73 response element comprises the sequence RRRCWWGYYY (SEQ ID NO: 7), wherein R is a purine, W is A or T, and Y is a pyrimidine.

Detail 57: A method of identifying a population of humans wherein an activation and/or a modulation of a p73 protein by an administering of a small-molecule switcher compound or a less than 1000 molecular weight switcher compound will provide a reduction of a c-FLIP-L/S gene expression which is operative to sensitize any cancer cells in the human population to a p73-induced apoptosis in the cancer cells, the method comprising the steps of: (1) obtaining a biological sample from each human in the population and/or obtaining data that has been previously derived from a biological sample from each human in the population, wherein the biological sample is a tumor biopsy, a blood sample, a urine sample, or a saliva sample, and wherein the data comprises gene expression data, protein expression data, or clinical data; (2) analyzing the biological samples and/or data to determine a level of p73 protein expression and/or activity in the cancer cells using an ELISA assay, a Western blot, immunohistochemistry, or a functional assay for p73 activity, and wherein the functional assay measures the transcriptional activity of p73 on a target gene; (3) identifying a subpopulation of humans having a low level of p73 protein expression and/or activity in the cancer cells compared to a control, wherein the low level is less than 50% of the level in the control, and wherein the control is a non-cancerous cell or tissue sample; wherein the identified subpopulation of humans is the population wherein an activation and/or a modulation of the p73 protein by the administering of the switcher compound will provide the reduction of the c-FLIP-L/S gene expression which is operative to sensitize the cancer cells to the p73-induced apoptosis.

Detail 58: The method of detail 57, wherein the switcher compound comprises small molecule CB-7587351, small molecule RETRA, small molecule Prodigiosin, small molecule NSC59984, or a combination thereof; and/or a pharmaceutically acceptable salt, hydrate and/or a solvate thereof, and wherein the switcher compound has a binding affinity for p73 with a dissociation constant (Kd) of less than 1 μM.

Detail 59: The method of detail 57, wherein the switcher compound is a modulator of the p73 either in addition to being an activator and/or instead of being an activator, wherein the modulator enhances the stability, half-life, or binding affinity of the p73 protein by inhibiting MDM2-mediated degradation of p73 or by promoting the acetylation of p73.

Detail 60: The method of detail 57, wherein the switcher compound activates p73 as an orthosteric activator by directly binding to the p73 protein at the DNA binding domain and/or as an allosteric activator by binding to a site distinct from the p73 active site and enhancing the activity of the p73 protein by inducing a conformational change.

Detail 61: The method of detail 57, wherein the switcher compound activates p73 which transcriptionally regulates the c-FLIP gene and/or an expression of the c-FLIP gene by binding to a p73 response element in the c-FLIP gene promoter, and wherein the p73 response element comprises the sequence RRRCWWGYYY (SEQ ID NO: 7), wherein R is a purine, W is A or T, and Y is a pyrimidine.

Detail 62: The method of detail 42, further comprising administering to the subject a second therapeutic agent selected from the group consisting of a chemotherapeutic agent, a radiotherapeutic agent, a targeted therapeutic agent, an immunotherapeutic agent, and combinations thereof, and wherein the chemotherapeutic agent is selected from the group consisting of an alkylating agent, an antimetabolite, an anthracycline, a plant alkaloid, a topoisomerase inhibitor, and an antitumor antibiotic.

Detail 63: The method of detail 62, wherein the second therapeutic agent is administered concurrently with, prior to, or subsequent to the administration of the p73 protein activator, and wherein the second therapeutic agent is administered in a therapeutically effective amount.

Detail 64: The method of detail 42, further comprising monitoring the subject for a response to the treatment, wherein the monitoring comprises imaging the cancer using CT, PET, MRI, or ultrasound, assessing cancer biomarkers in a biological sample, or evaluating symptoms of the cancer using RECIST or WHO criteria.

Detail 65: The method of detail 64, further comprising adjusting the amount, frequency, or duration of the administration of the p73 protein activator based on the response to the treatment, wherein the adjusting comprises increasing the amount, frequency, or duration if the response is not satisfactory, or decreasing the amount, frequency, or duration if the subject experiences adverse effects.

Detail 66: A kit comprising: a) a p73 protein activator, wherein the p73 protein activator is a switcher compound selected from the group consisting of: 2-[(E)-2-(3,4-dihydroxyphenyl)ethenyl]-1-benzofuran-6-ol (CB-7587351); 2,5-bis(5-hydroxymethyl-2-thienyl)furan (RETRA); 4-methoxy-5-[(Z)-2-pyridin-3-ylvinyl]-1H-pyrrole-2-carbaldehyde (Prodigiosin); 2-[(E)-2-(4-nitrophenyl)ethenyl]-1-benzofuran-5-ol (NSC59984); and/or a pharmaceutically acceptable salt, hydrate and/or a solvate thereof; and b) instructions for administering the p73 protein activator to a subject with cancer, or a subject suspected of having cancer, or a subject who is susceptible to cancer, for treating and/or preventing the cancer in the subject, wherein the instructions comprise information about the dosage, route of administration, and duration of treatment.

Detail 67: The kit of detail 66, further comprising a second therapeutic agent selected from the group consisting of a chemotherapeutic agent, a radiotherapeutic agent, a targeted therapeutic agent, an immunotherapeutic agent, and combinations thereof.

Detail 68: The kit of detail 66, further comprising a device for administering the p73 protein activator, wherein the device is selected from the group consisting of a syringe, an inhaler, a nebulizer, a spray bottle, a dropper, and a patch.

Detail 69: The kit of detail 66, further comprising a biomarker detection assay for detecting the level of p73 protein expression and/or activity in a biological sample from the subject.

Detail 70: The kit of detail 66, wherein the p73 protein activator is formulated in a dosage form selected from the group consisting of a tablet, a capsule, a solution, a suspension, an emulsion, a powder, a granule, a suppository, an injection, an infusion, an inhalation, a spray, a lotion, an ointment, a cream, a gel, a patch, and a sustained-release formulation.

Detail 71: The kit of detail 66, wherein the p73 protein activator is co-packaged with the instructions, the second therapeutic agent, the device, and/or the biomarker detection assay.

Significance: The incidence of cancer is rising and the failure, despite many years of scientific efforts, to find a cure has led to extreme urgency for the technology disclosed herein.

### EXAMPLES

Example abstract: The tumor suppressor p73 is a member of the p53 family and transcriptionally activates multiple p53-targets involved in cell cycle regulation and apoptosis. In addition to pro-apoptotic signaling, outcomes of p73 activation include cell survival signals. Thus, p73 activity and targets may provide insight into cell fate outcomes between cell survival and apoptosis following cellular stress. Herein we report that cellular FLICE inhibitory protein (c-FLIP), a master anti-apoptotic factor, is a transcriptional target of p73.

### Example 1. Experimental Approach, to Identify One Example Small Molecule CB-7587351 as a “Switcher Compound”

The activation of p73 (α and β isoforms) transcriptionally upregulates c-FLIP-L/S expression in cancer cells. The cell fate decision following p73 activation is determined by the adjustment of the balance of outcomes of p73 activation between p73-induced pro-apoptotic signaling and c-FLIP-L/S expression in cancer cells. p73 primes extrinsic apoptosis via an autocrine death ligand-DR5 axis, and the priming appears to be titrated at the level of c-FLIP-L/S. The p73-upregulation of c-FLIP-L/S increases the threshold of extrinsic apoptosis. Cells with poor priming levels convert to cell cycle arrest and survival. Depletion of c-FLIP-L/S increases the p73-priming levels towards extrinsic apoptosis and sensitizes cancer cells to p73-primed extrinsic apoptosis. We further identified a small-molecule CB-7587351 (“switcher compound”) that alters p73 activation outcomes through c-FLIP-L/S protein degradation. Therapeutic activation of p73 can restore p53-signaling in mutant p53-expressing cancer cells effectively bypassing the p53 deficiency in cancer cells. Our discovery of p73 transcriptional upregulation of c-FLIP provides a promising strategy for depleting c-FLIP to improve antitumor efficacy of p73-targeting cancer therapy for p53-mutant tumors.

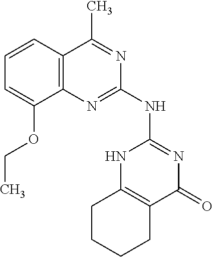 [SMILES: CCOc1cccc2c(C)nc(Nc3nc(=O)c4c([nH]3)CCCC4)nc12]

CB-7587351 non-salt form (no charge shown), additional hydrate and/or solvate not shown 2-(8-Ethoxy-4-methyl-2-quinazolinylamino)-5,6,7,8-tetrahydro-4 (1H)-quinazolinone, or 2-(8-ethoxy-4-methylquinazolin-2-ylamino)-5,6,7,8-tetrahydroquinazolin-4 (1H)-one.

Introduction: As discussed above, the tumor suppressor p73 is a family member of p53, a guardian of genome integrity through repair of DNA and regulation of the cell cycle. p73 and p53 have high structure and sequence homology. Like p53, p73 transcriptionally activates a subset of p53-targeted genes, leading to cell cycle arrest, cellular apoptosis and chemo-sensitivity [1, 2]. p73 loss contributes to tumor development [3-6]. Therefore, p73 is considered as a tumor suppressor. Unlike p53 which is frequently mutated in approximation of 50% tumors [7], p73 is rarely lost or mutated in cancer cells. Therefore, p73 appears to be a promising target to reinforce p53 pathway signaling for cancer therapy in p53-mutant or p53-null cancer cells [8].

Our laboratory previously reported that approximately half of the small molecules in the selected leads from the National Cancer Institute (NCI) diversity set library restored p53 pathway signaling through activation of p73 [9]. Small molecules, such as RETRA, Prodigiosin and NSC59984, activate p73 via releasing it from the inhibitory complex of mutant p53 [10-12]. These studies support a rationale for therapeutic activation of p73 to bypass and restore wild-type function to mutant p53 in cancer cells.

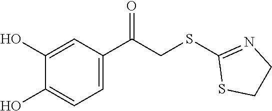 [SMILES: O=C(CSC1=NCCS1)c1ccc(O)c(O)c1]

RETRA, non-salt form (no charge shown) a/k/a mutant p53 reactivator, 2-(4,5-Dihydro-1,3-thiazol-2-ylthio)-1-(3,4-dihydroxyphenyl)-1-ethanone, or 2-(4,5-dihydro-1,3-thiazol-2-ylthio)-1-(3,4-dihydroxyphenyl) ethan-1-one.

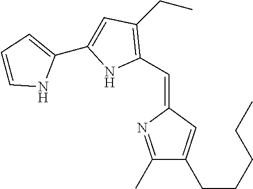 [SMILES: CCCCCC1=C/C(=C/c2[nH]c(-c3ccc[nH]3)cc2CC)N=C1C]

Prodigiosin (non-salt form) or 4-Ethyl-5-[(Z)-(5-methyl-4-pentyl-2H-pyrrol-2-ylidene)methyl]-1H,1′H-2,2′-bipyrrolyl, or 4-ethyl-5-[(1Z)-(5-methyl-4-pentyl-2H-pyrrol-2-ylidene)methyl]-1H,1′H-2,2′-bipyrrole.

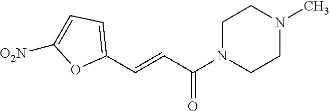 [SMILES: CN1CCN(C(=O)/C=C/c2ccc([N+](=O)[O-])o2)CC1]

NSC59984 (non-salt form), CAS 803647-40-7, a/k/a p53-MDM2 inhibitor, or (E)-1-(4-Methyl-1-piperazinyl)-3-(5-nitro-2-furyl)-2-propen-1-one, or (2E)-1-(4-methylpiperazin-1-yl)-3-(5-nitrofuran-2-yl)prop-2-en-1-one.

However, there are no FDA approved p73-targeting compounds in clinical trials, and the therapeutic efficacy of p73 activators needs to be improved. p73 has more than an anti-tumor effect as it has also been found to regulate development among other functions. p73 plays a

critical role in maintaining neuron cell survival [13], and sustains cell stemness of cancer stem-

like cells through redox metabolic reprograming [14]. A global gene expression analysis has shown that p73 activation included, but was not limited to, proapoptotic and survival signal

patterns [15]. There is an unmet need for understanding how p73 determines the cell fate between cell survival and cellular apoptosis in response to cellular stresses. Answers to this question will be helpful to improve the anti-tumor efficacy of targeting p73.

Apoptosis is one of the major types of cell death and occurs through intrinsic and extrinsic pathways. The intrinsic pathway is initiated by intracellular stress through mitochondria,

and the extrinsic pathway is triggered by external stimuli through cell death receptors [16]. The key factors involved in activating the two pathways, such as DR5 in the extrinsic pathway and PUMA in the intrinsic pathway, are transcriptional targets of p53 and p73 [8, 17]. Both pathways are considered as a predominant mechanism by which p53 and p73 induce cell apoptosis. Cellular FADD-like IL-1 beta-converting enzyme (FLCE)-inhibitory protein (c-FLIP) is an anti-apoptotic factor playing a key role in controlling the extrinsic apoptotic pathway by blocking the activation of caspase 8. The c-FLIP family comprises three isoforms at the protein level, c-FLIP-L (long isoform), c-FLIP-S (short isoform) and c-FLIP-R (Raji isoform), and all of them lack caspase activity therefore inhibit caspase 8 autoactivation when they are bound together [18, 19]. Recently, p53 and its other family member p63 both have been found to upregulate c-FLIP-L expression in cancer cells [20-22].

The p53-upregulation of c-FLIP-L partially leads to cancer cell resistance to Nutlin-mediated induction of p53-dependent apoptosis, but the resistance mechanism remains unclear [22]. A recent study reported a correlation between the increase of c-FLIP at the protein level and the overexpression of the TAp73a in hepatocellular carcinoma cells Hep3B and HepG2 [23]. There is a need to understand the role of the upregulation of c-FLIP in the cell fate decision within the p53 family in order to increase the antitumor efficacy of targeting the p53 family accordingly.

In this technology, we explored and described the role of p73 (both α and β isoforms) as a transcription factor in upregulating c-FLIP-L/S expression in cancer cells. The p73-dependent upregulation of c-FLIP, in turn, results in poor priming of extrinsic apoptosis in cancer cells when p73 is activated. p73 appears pivotal in a cell fate decision by balancing its outcomes between c-FLIP-L/S expression and pro-apoptotic signaling in cancer cells. This insight was further exploited to identify a small molecule CB-7587351 as a “switcher compound” to adjust the outcome of p73 signaling through degradation of c-FLIP and activation of the p73 pathway in cancer cells. Our study develops a new strategy for harnessing c-FLIP-L/S depletion to increase the antitumor efficacy of targeting p73 in TP53-deficient cancer cells for cancer therapy.

Materials and Methods; High-Throughput Screening: Functional cell-based screening for small molecules that increase p53-transcriptional activity was performed as described in the previous study using noninvasive bioluminescence imaging in human colorectal cancer cells which stably express a p53 reporter, PG13-luc [10]. Briefly, cells were treated with 10 μM of compounds from a Chembridge library (50K small molecules), and p53 transcriptional activity was evaluated by bioluminescence from a p53-reporter in cells using an IVIS imaging system (Xenogen, Alameda, CA) at 2 h, 24 h and 72 h after compound treatment. DMSO treatment was used as a negative control in each screened plate. Compounds that increased p53-responsive bioluminescence were selected for secondary screening.

CellTiter-Glo luminescent Cell viability assay: Cells were seeded on 96-well plates with a density of 3000 cells/well and treated with small molecule compounds as desired for 72 hours. Cell viability was measured by CellTiter-Glo bioluminescence (Promega, catalog no. G7572), and analyzed using an IVIS imager.

Annexin V Apoptosis Assay: Cells were seeded on 96-well black plate, and treated with the reagents as indicated in the figures (see FIGs.). Annexin V levels were measured using RealTime-Glo™ Annexin V assay kit (Promega). The bioluminescent Annexin V was analyzed using an IVIS imager.

Chromatin Immunoprecipitation (ChIP) PCR assay: ChIP was performed according to the protocol from Upstate Biotechnology with slight modifications as previously described [24]. Briefly, 2×10⁶ cells were fixed with 1% formaldehyde, followed by sonication which sheared the cross-linked DNA fragments to 200-1000 base pairs in length. The sonicated chromatin was incubated with 3 μg of anti-p73 antibody (A300-126A, Bethyl Laboratories) at 4° C. overnight, and 50 μl of packed salmon sperm DNA/protein A-Sepharose beads at 4° C. for 3 hr. The precipitated beads were washed with different washing buffers [24]. CHIP-eluted DNA was reversed and extracted with phenol/chloroform. The eluted DNA was analyzed by quantitative Real-Time PCR with the following primers for ChIP [25]:

Primers for the c-FLIP promoter: c-FLIP promoter region #1: forward primer 5′-TTAAATGCCTGCCCCTACTG-3′ (SEQ ID NO: 1), reverse primer 5-TAGCACCTCCATCACCACCT-3′ (SEQ ID NO: 2); c-FLIP promoter region #2: forward primer 5′-AAGAGAATCGCTTGAACTAGGAAGG-3′ (SEQ ID NO: 3), reverse primer 5′-CTATGGCTTGTGTGACTGAGTATGC-3′ (SEQ ID NO: 4); c-FLIP promoter region #3: forward primer 5′-TTACCTTCAGCATCAGGTAGCTAGG-3′ (SEQ ID NO: 5), reverse primer 5′-CTCTTGGATCAGAATGTGAGAGTCA-3′ (SEQ ID NO: 6).

Western Blot analysis: An equal amount of protein in the cell lysate was loaded in each well and electrophoresed through 4-12% SDS-PAGE then transferred to a PVDF membrane. The primary antibodies indicated in the FIGs. were incubated with the PVDF membrane after transfer in blocking buffer at 4° C. overnight. Antibody binding was detected on PVDF with appropriate IR Dye-secondary antibodies (LI-COR Biosciences, USA) by the ODYSSEY infrared imaging system or with ECL Reagent (Thermo Fisher Scientific, catalog no. 32106) chemiluminescence reaction with appropriate horseradish peroxidase (HRP)-conjugated secondary antibodies (Thermo Fisher Scientific, catalog no. no. 31460 for Goad anti-rabbit IgG and catalog no. 31430 for Goat anti-mouse IgG) by the Syngene imaging system.

c-FLIP promoter-driven luciferase reporter assay: c-FLIP-promoter-luciferase plasmids were generated previously in the lab [26]. The FLIP-promoter-luciferase plasmids were transiently transfected with lipofection 2000 (Life Technologies, catalog no. 11668-027) into cancer cells as stated in the FIGs. for 24 hours, followed with Adenovirus-p73 infection for 24 hours. Luciferase reporter activity in cells was measured based on the bioluminescence using IVIS after addition of luciferin.

Knockdown of gene expression by siRNA: Cells were transfected with siRNA using lipofectamine RNAiMAX (Life Technologies, catalog no. 13778075) as described in the protocol from the manufacturer. At 48 hr after transfection, cells were further treated as indicated in the FIGs.

Flow Cytometry Assay: Briefly, cells were fixed with 70% ethanol, and stained with Propidium Iodide (PI), then subjected for CytoFLEX to measure DNA content of the PI-stained cells. Cell cycle distribution was analyzed with Flowjo.

Cytokine profiling assay: Cell culture supernatant levels of the cytokine indicated in the supplementary table 1 were measured with a Human Premixed Multi-Analyte Kit (R&D Systems, Inc., Minneapolis, MN) using a Luminex 200 Instrument (LX200-XPON-RUO, Luminex Corporation, Austin, TX) according to the manufacturer's instructions. Analyte values were reported in picograms per milliliter (pg/mL). Any values that say “<X” mean the sample value for that analyte was below the lower limit of detection, and any value that says “>X” means the sample value for that analyte was above the upper limit of detection. Any values that say “<X” was recoded as zero in this study.

Statistical analysis: Statistical analyses were performed using the student's t-test or Anova with GraphPad Prism software. Statistical significance was determined by p<0.05. Combination indices were calculated using the Chou-Talalay method with CompuSyn software.

Results; The c-FLIP gene is transcriptionally regulated by the tumor suppressor p73 protein: To determine whether p73 regulates c-FLIP-L/S expression in cancer cells, we transiently overexpressed p73-αand p73-β, the two p73 active isoforms, in cancer cells using lipofectamine transfection and adenovirus infection, respectively. Both the overexpression of p73-α and p73-β dramatically increased c-FLIP-L/S at the protein level in various cancer cell lines carrying either wild-type p53 or mutant p53 (FIG. 1A and FIG. 1B, FIG. 9A and FIG. 9B).

To further examine the c-FLIP-L/S expression in TP53-deficient cells as compared to wild-type p53 expressing cells with same genetic background, we generated p53-knockout (KO) U2OS using CRISPR and established stable expression of mutant p53-R175H in the U2OS cell line. The knockdown of p73 expression in these three cell lines reduced c-FLIP-L/S expression at the protein level as compared to siRNA control (FIG. 1C). The same effect of the knockdown of p73 on c-FLIP expression was also observed in other p53-mutant cancer cells (FIG. 9A and FIG. 9B). These results, taken together, suggest p73-dependent c-FLIP-L/S expression in cancer cells regardless of p53 status.

To examine if p73 transcriptionally upregulates c-FLIP expression, we performed a ChIP-PCR analysis using the primers flanking three different regions of the c-FLIP promoter and further did c-FLIP promoter-driven luciferase reporter assays accordingly (FIG. 1D). The ChIP-PCR assay detected the c-FLIP promoter regions bound with endogenous p73 in p53-mutant cancer cells (FIG. 1E), and further revealed an increase in c-FLIP promoter bound with the recombinant p73-β in a p73-level dependent manner in the cancer cells (FIG. 1F). These results, taken together, suggest the binding of p73 to the c-FLIP promoter in cancer cells. Further mechanistic studies using c-FLIP promoter-driven luciferase reporter assay showed that both p73-β and p73α increased the c-FLIP-promoter (PR4 and PR5)-Luciferase activity with the highest PR5-luc upon the p73 overexpression in U2OS, P53-KO-U2OS or mutant-p53 R175H expressing cancer cells (FIG. 1G and FIG. 1H). PR 1500-luc reporter assay showed a significant increase in luciferase activity upon p73α overexpression as compared to the overexpression of p73-β in the cells (FIG. 1G and FIG. 1H). These results, taken together, suggest that p73 transcriptionally upregulates c-FLIP expression in cancer cells.

Reduction of c-FLIP-L/S expression sensitizes cancer cells to p73-induced apoptosis in cancer cells: To assess the p73-dependent cell fate decision between cell survival and death, we performed a cell cycle assay and found that 5-10% of cells were detected in the subG1 phase (FIG. 2A), consistent with the observation of no increase in cleaved-PARP, a cell death marker, in the cells following p73-β overexpression for 2 days (FIG. 1A). Moreover, cell cycle analysis showed ˜60% of the p73-overexpressing cells in the G1 phase as compared to the 40% of the control cells (FIG. 2B). Consistent with the cell cycle profiling, the level of phospho-Rb was reduced, and cyclin D1 was increased, both of which were correlated to increased p21 protein in cells with p73-β overexpression (FIG. 1A). These results suggest cell survival along with cell cycle arrest in cells upon the p73 overexpression under the experimental conditions. To further examine the effect of p73 on cell death phenotypes, we extended the duration of p73 overexpression from 48 hours to 72 hours in tumor cells and found an increase of cell number in the sub-G1 phase of the cell cycle in cancer cells with the extended duration of p73 overexpression to 72 hours (FIG. 2C).

To examine if upregulation of c-FLIP inhibits p73-induced cell apoptosis, we knocked down c-FLIP-L/S expression in cancer cells. In the knocked-down c-FLIP-L/S cells, the overexpression of p73 rapidly increased cell number in the sub-G1 phase at 30 hours (FIG. 2D), consistent with the enhanced cell death markers cleaved-PARP, cleaved caspase-8 and cleaved caspase-3 in the cells with overexpression of either p73 α or p73 β (FIG. 2E and FIG. 2F). To examine which one of the c-FLIPL/S proteins plays a significant role in attenuating p73-mediated cell apoptosis, we further selectively knocked down c-FLIP-L or c-FLIP-s using specific siRNA in the cancer cells. The knockdown of c-FLIP-L or c-FLIP-s increased PARP cleavage, the cell death marker in cancer cells (FIG. 2F). Bioluminescent Annexin V analysis showed an increase in bioluminescence in the Adp73-infected cells transfected with siRNAs either targeting c-FLIP-L or c-FLIP-s, or both, indicating cell death (not shown). These results suggest that c-FLIPL/S is a major factor involved in p73-mediated cell fate determination, i.e., c-FLIP-L/S loss promotes p73-induced cell apoptosis.

p73 induces extrinsic apoptosis in c-FLIP-deficient cancer cells: To examine the apoptotic pathway by which p73 induces cell death, we knocked down caspase-8 (a major mediator in the extrinsic pathway) and caspase-9 (a major mediator in the intrinsic pathway) in cancer cells. The knockdown of caspase-8 or caspase-9 partially rescued the cell death in the cells with siRNA control in response to the p73 overexpression (FIG. 3A), suggesting that the longer exposure to p73 activation causes cell death partly via the intrinsic and/or extrinsic apoptotic pathways. By contrast, the effect of the p73 on cell death was blocked mainly by the knockdown of caspase-8 in the c-FLIP knockdown cancer cells based on the sub-G1 flow cytometric assay (FIG. 3A). We further found that knockdown of caspase-8 blocked PARP cleavage in the c-FLIP-knockdown cancer cells in response to either p73-β or p73-α overexpression, but the knockdown of caspase-9 could not do so in cells with treatment for 30 hours (FIG. 3B, FIGS. 10A-10C). These results suggest that p73 induces cell death mainly via extrinsic apoptosis in c-FLIP-deficient cells.

TRAIL Death Receptor DR5 is an upstream cellular factor in the extrinsic cell death pathway. To further examine if the extrinsic pathway is required in this process with a short expose of p73 overexpression in the c-FLIP-knockdown cells, we knocked down DR5 and FADD, the components of the Death-Inducing-Signaling-Complex (DISC). The knockdown of either DR5 or FADD partially blocked PARP-cleavage in the c-FLIP-knockdown cancer cells upon p73-β overexpression at 30 hr (FIG. 3C). By contrast, knockdown of PUMA, a factor involved in the intrinsic pathway, could not block p73-induced cleavage of PARP in the c-FLIP KD cells (FIG. 3D). Activation of caspase-8 may result in cleavage of Bid, a Bcl-2 family protein with a BH3 domain only, which, in turn, translocates to mitochondria to release cytochrome c thereby initiating a mitochondrial amplification loop [27]. The pro-apoptotic Bid connects the activation of the extrinsic death receptor pathway to activation of the mitochondrial-disruption processes associated with the intrinsic pathway. The knockdown of Bid could not attenuate the cleavage of PARP or caspase-9 in c-FLIP-knockdown cells (FIG. 3D).

To identify if cell death ligands engage in the effect of p73 activation of apoptosis, we further examined cytokine secretion from cancer cells upon p73 overexpression. We transiently transfected p73-α into cells using lipofectamine. The cytokine profiling showed an increase of cell death ligands such as TNF and TRAIL in the cell culture media upon the p73-α overexpression in the cancer cells (data not shown).

Small-molecule CB-7587351 “switcher compound” alters p73 outcomes by upregulation of proapoptotic signaling and reduction of c-FLIP in p53-mutant cancer cells

We further applied the above insights to search for small-molecule compounds altering outcomes of p73 activation between proapoptotic signaling and c-FLIP levels for cancer therapy. We screened a Chembridge library of small-molecule compounds that restored p73 signaling via a high-throughput screen and examined c-FLIP protein expression in cancer cells treated with hits from the library. We identified small molecule CB-7587351 (e.g., 2-[(8-ethoxy-4-methyl-2-quinazolinyl)amino]-5,6,7,8-tetrahydro-4 (1H)-quinazolinone) that reduced c-FLIP protein expression in p53-mutant and p53 wild-type cancer cells (FIG. 4A and FIG. 4B). The Real-time PCR assay showed no significant changes of c-FLIP expression at the mRNA level in the cancer cells treated with CB-7587351 (FIG. 4C), but the western blot assay showed a decrease in c-FLIP-L/S at the protein level in the cancer cells (FIG. 4B and FIG. 4D). Furthermore, CB-7587351 treatment reduced p73-mediated upregulation of c-FLIP protein in cancer cells with overexpression of p73 (FIG. 4D). To examine if treatment with CB-7587351 reduces c-FLIP protein stability, cancer cells were treated with MG132, a proteasome inhibitor, the treatment with MG132 rescued c-FLIP from the CB-7587351-induced protein degradation in the cancer cells (FIG. 4E).

These results suggest that CB-7587351 induces c-FLIP protein degradation and prompted us to define it as a “switcher compound” as the consequence is an impact on cell fate from cell survival to cell death. We further knocked down C-terminus of Hsc70-Interacting Protein (CHIP) and ITCH, two E3 ligases that have been reported to be involved in c-FLIP degradation in cancer cells [28, 29]. The knockdown of either CHIP or ITCH could not rescue c-FLIP from the CB-7587351-induced protein degradation in the cancer cells (FIG. 4F and FIG. 4G). We also treated cells with caspase pan inhibitors or siRNA to silence caspase 8 in cells, and the cells with the inhibition of caspases showed the degradation of c-FLIP in response to the CB7587351 treatment (FIG. 4F, FIGS. 11A-11D), suggesting that the c-FLIP reduction is not due to cell death at the early time points.

In addition to the above observations, small molecule CB-7587351 restores p53 pathway signaling in cancer cells as a bone fide p53 pathway restoring compound (FIGS. 5A-5F). CB-7587351 strongly induced p53-responsive bioluminescence in different cell lines SW480 (mutant p53 R271H and P309S), DLD1 (mutant p53 S241F) and HCT116 p53-/-colorectal cancer cells as well as in wild type p53-expressing cancer cells (HCT116) in a dose dependent manner regardless of p53 (FIG. 5A). Endogenous p53 targets were increased at the protein level by CB-7587351 treatment in p53-mutant and wild colorectal cancer cell lines (FIG. 4D, FIG. 5B).

A real-time RT-PCR assay showed a slight increase of the selected p53 targets at the mRNA level in the cells treated with CB-7587351 (FIG. 5C). These results suggest that CB-7587351 restores p53 pathway signaling in cancer cells carrying wild-type p53 or mutant p53. To examine if p73 is involved in the CB-7587351-reactived p53 pathway in mutant p53-expressing cancer cells, we overexpressed recombinant p73-β with adenovirus infection and observed the effect of CB-7587351 on the PG13-luc reporter to be further enhanced by the overexpression of p73-β as well as wild-type p53 (FIG. 5D and FIG. 5E). The knockdown of p73 blocked the activation effect of CB-7587351 on p21 and puma expression in the DLD-1 cells (FIG. 5F). The transient transfection of siRNA-targeting p73 attenuated the CB7587351-induced PG-13-lucifease reporter in p53-KO cancer cells (FIG. 5G). These results suggest that CB-7587351 restores p53 pathway signaling via activation of p73 in p53-mutant or p53-null cancer cells.

We examined whether DNA damage engages in the mechanism of the action of CB-7587351 on p53 signaling restoration (FIGS. 12A-12C). The foci and levels of the phosphorylation of γ-H2AX, a sensitive indicator for DNA damage due to DNA double-strand breaks, were measured in the cancer cells. No phosphorylation of y-H2AX was found in SW480 cells in response to an increased dose of CB-7587351 during early time points following CB-7587351 treatment (FIGS. 12A-12C). Moreover, no DNA-intercalation was detected (not shown). These results, taken together, suggest little or no genotoxicity of CB-7587351.

### CB-7587351 Induces Extrinsic Apoptosis in Mutant-p53 Expressing Cancer Cells

To evaluate the therapeutic index of CB-7587351, we examined cell viability of a panel of cancer cell lines with CB-7587351 treatment. The IC50s of CB-7587351 were much lower in cancer cells than those in normal fibroblast MRC-5 cells (FIG. 6A), indicating a favorable therapeutic index for CB-7587351. A flow cytometric assay showed an increase in sub-G1 content in cancer cells, but not in normal fibroblast cells MRC-5 after CB-7587351 treatment (FIG. 6B). In addition, CB-7587351 treatment dramatically reduced colony formation in colorectal cancer cells (FIG. 6C). Knockdown of p73 abrogated the effect of CB-7587351 on sub-G1 content and PARP cleavage in DLD-1 cells (FIG. 6D and FIG. 6E), and the overexpression of p73 enhanced the anti-tumor effect of CB-7587351 based on increased sub-G1 content and Annexin V levels (FIG. 6F and FIG. 6G).

An increase of cleaved caspase-3 and caspase-3/7 activity was detected in cancer cells treated with CB-7587351 at early time points (FIG. 7A and FIG. 7B). Treatment with z-VAD-FMK, a pan-caspase inhibitor, blocked the effect of CB-7587351 on apoptosis in SW480, HT29, HCT116 p53-/- and HCT116 colorectal cancer cells according to the sub-G1 assay (FIG. 7C and FIG. 7D). We further examined which one, the extrinsic or intrinsic apoptotic pathway, is involved in the induction of the apoptosis. Knockdown of caspase-8 prevented apoptosis induced by CB-7587351 at 10 μM based on the lack of cleavage of PARP and Annexing-V levels, but knockdown of caspase-9 could not (FIG. 7E and FIG. 7F). These results suggest that CB-7587351 induces extrinsic apoptosis in p53-mutant cancer cells. Knockdown of caspase 9 showed a lesser extent of cleaved caspase 3 as compared to the siRNA control in the cells treated with CB7587351 (FIG. 7E). These results suggest that other caspases might also be involved in the process.

As TRAIL is a major death ligand driving cell death via extrinsic apoptosis and the innate immune system, we observed that CB-7587351 treatment synergized with TRAIL to reduce cell viability in cancer cells, but not in normal cells at the tested doses (FIG. 8A and FIG. 8B). CB7587351 treatment enhanced TRAIL-induced PARP cleavage in cancer cells (FIG. 8C, FIGS. 13A-13C). The combinational effect on PARP cleavage was blocked by knockdown of caspase-8(FIG. 8D). These results suggest that CB-7587351 sensitizes cancer cells to TRAIL-induced cell apoptosis via extrinsic apoptosis.

On the basis of these findings, we propose that CB-7587351 “switch compound” alters the p73 outcome cell fate balance via degradation of c-FLIP, promoting cells to undergo p73-primed extrinsic apoptosis (FIG. 8E).

Discussion: Current approaches for targeting p73 are mainly focused on activating p73 proapoptotic signaling, which can compensate for wild-type p53 deficiency in p53-mutant cancer cells, to induce cell death for cancer therapy [9-12]. However, outcomes of p73 activation are complex, and this can influence the anti-tumor efficacy of p73-targeting therapeutic approaches. There is a clear need to understand mechanisms by which p73 determines cell fate between growth arrest (cell cycle arrest or senescence), pro-survival phenotypes, or cell death including apoptosis. We demonstrate here that p73 transcriptionally activates the c-FLIP-L/S gene that encodes an an anti-apoptotic factor. The upregulation of c-FLIP-L/S, in turn, results in a low level of p73-priming in extrinsic apoptosis that promotes cell survival rather than cell death. Targeting c-FLIP-L/S protein degradation with the “switcher compound” approach we describe leads cancer cells towards a p73-induced extrinsic apoptotic cell fate.

p73-mediated transcriptional upregulation of c-FLIP-L/S promotes cellular tolerance to p73-activated apoptotic signaling, given that the knockdown of c-FLIP-L/S sensitizes cells to p73-induced apoptosis (FIGS. 2A-2F and FIGS. 3A-3E). Similar to p73, p53 activation has been

reported to upregulate c-FLIP-L, which prevents p53-induced apoptosis [22]. Paradoxically, while p73 upregulates c-FLIP protein expression, p73 also increases DR5 expression and TRAIL cell death ligand secretion, key factors activating the extrinsic apoptotic (FIGS. 3A-3E). Therefore, we hypothesize that p73 primes the extrinsic apoptotic pathway via a cell death ligand-receptor axis, and the priming level is titrated by the upregulation of c-FLIP-L/S (FIG. 8E).

The extrinsic apoptotic pathway involves engagement of multiple death receptors through formation of a DISC which contains core cell death signaling components such as FADD, DR5 and caspase-8. The DISC initiates a cascade activating caspase-8 and downstream caspase-3 when the receptors are bound with different death ligands such as tumor necrosis factor (TNF) and Fas [30]. Both DR5 and FADD are required for p73 to induce cell death in our experiments with c-FLIP-knockdown cells generated during our study (FIGS. 3A-3E), indicating that p73 primes extrinsic apoptosis but attenuates it through c-FLIP. Moreover, the cell death ligands, TNF and TNF-related apoptosis-inducing ligand (TRAIL) are secreted by cells following overexpression of p73 (FIG. 3E), in accordance with the previous reports that have shown that p73 activation increases expression of TNF family members in cells [31]. Like p73, p53 has been reported to increase Fas expression and enhance levels of Fas at the cell surface [32]. In addition, p21 (WAF1), the target of p73 and p53, can also increase expression of cytokines at mRNA levels including Fas [33]. These discoveries suggest a potential autocrine regulation of ligands through CD95 (APO-1/Fas) or TRAIL receptor (DR5) involved in p73-priming of extrinsic apoptosis. Death ligands such as TNF or TRAIL promote activation of the initiator caspase-8 through DISC formation that can propagate the apoptotic signal [34]. As c-FLIP-L/S has sequence homology to caspase-8 and caspase-10 but lacks catalytic activity, c-FLIP-S recruitment to the DISC blocks caspase-8 or caspase-10 caspase activation [35]. c-FLIP-L is a pseudo-caspase and regulates caspase-8 activity based on their ratio in forming heterodimers at the DISC [36]. High levels of c-FLIP-L lead to predominant heterodimer formation with caspase-8 and inhibits caspase-8 activation and apoptosis [19, 36]. Therefore, p73-transcriptional upregulation of c-FLIP-L/S may impede p73-primed extrinsic apoptosis via inhibition of caspase-8 activation (FIG. 8E). Indeed, knockdown of c-FLIP increases caspase 8 cleavage in response to p73 overexpression (FIGS. 2A-2F and FIGS. 3A-3E). The upregulation of c-FLIP-L and c-FLIP-S attenuates p73 priming of extrinsic apoptosis, (FIG. 2F). The poor p73-priming of extrinsic apoptosis (attenuated by c-FLIP) along with cell cycle arrest (regulated by p21 (WAF1) that is induced by p73) can confer cells to endure harsh conditions of stress and survive in response to p73 activation. Indeed, the upregulation of c-FLIP is correlates with p21 expression in response to p73 activation (FIGS. 2A-2F), and depletion of c-FLIP-L/S increases extrinsic apoptosis correlated with reduced expression of p21 in cells with p73 overexpression (FIGS. 2A-2F). These results suggest that the high priming level of extrinsic apoptosis converts to cell death rather than survival (cell cycle arrest) upon p73 overexpression (FIG. 8E) in a c-FLIP-L/S-dependent manner.

Intrinsic apoptosis appears to be another predominant mechanism by which p73 induces cell death. The signaling involved in intrinsic apoptosis is enhanced by p73 overexpression in tumor cells (FIG. 3D), but no or less cell death was detected at early time points of 30-48 hr (FIGS. 2A-2F). Intrinsic apoptosis occurs through mitochondrial, and mitochondrial apoptosis priming is another key determinant of cell fate [37]. Extended duration (72 hr) of p73 overexpression increased cell death partially via the intrinsic apoptotic pathway in cancer cells with the poor levels of p73-primed extrinsic apoptosis (as compared to the shorter exposures of 30-40 hr) (FIG. 3A and FIGS. 10A-10C). By contrast, p73 rapidly induces cell death via extrinsic apoptosis in cancer cells when the priming level of extrinsic apoptosis is increased by knockdown of c-FLIP (FIG. 2D-2G, FIGS. 3A-3E). We did not measure mitochondrial apoptosis priming in this study. We hypothesize that the priming levels of both intrinsic and extrinsic pathways may have thresholds, and that the cellular apoptotic pathway used is determined by which threshold is breached through first. Depletion of c-FLIP may lower the threshold, resulting in highly primed cells readily undergoing p73-induced extrinsic apoptosis. The poor priming of extrinsic apoptosis by the upregulation of c-FLIP can protect cells from p73-primed and autocrine cell death ligand-driven extrinsic apoptosis. p73 signaling and cell fate can be manipulated with enhanced intensity and extended duration to overcome the c-FLIP-mediated cell survival threshold.

We demonstrate that p73 binds to the promoter of c-FLIP and transcriptionally upregulates c-FLIP expression in cancer cells (FIGS. 1A-1I), thus raising the interesting and important question of how p73 transcriptionally activates c-FLIP. There are multiple transcription factors involved in regulating c-FLIP expression, performing as either activators (such as NFκB and p53) or suppressors (such as c-Myc) [22]. The promoter regions tested in this study have been reported by us previously to be bound by c-Myc [26]. Interestingly, p73 has been found to directly interact with c-Myc to regulate gene expression [38, 39]. Whether p73 interrupts c-Myc's suppressive effect on c-FLIP expression should be examined in the future. Other potential transcriptional cofactors might also impact the function of p73 in regulating c-FLIP transcription in these regions, because the p73-β increases PR4-and PR5-luc reporter activities much more than what was observed with FLIP-1500-luc reporter (FIGS. 1A-1I). The detailed regulatory mechanisms by which p73 transcriptionally upregulates c-FLIP expression will be need to be more thoroughly addressed in the future, and will be helpful for pharmacological intervention targeting c-FLIP related transcription factors accordingly to increase p73-primed extrinsic apoptosis.

Our study indicates that c-FLIP is a critical factor for cell fate decisions in p73-primed extrinsic apoptosis. The depletion of c-FLIP using siRNA and the small-molecular “switch compound” CB-7587351 results in a high level of p73-primed extrinsic apoptosis, which sensitizes cancer cells to either p73 activation or TRAIL treatment (FIGS. 2A-2F and FIGS. 8A-8E). Because of the homology between caspase-8 and c-FLIP, inhibiting c-FLIP activity is a big challenge. Targeting c-FLIP degradation via E3 ligase-mediated proteolytic ubiquitination appears to be a promising approach for cancer therapy. CB-7587351 identified in this study provides an example of a small molecule with a dual function by depleting c-FLIP and activating p73 signaling to increase anti-tumor efficacy in p53-mutant cancer cells, though the specific immediate molecular targets of CB-758735 remain unclear. Given the effects of CB-7587351 on both p53/p73 activation and c-FLIP degradation, we speculate that the mechanism of action of CB-7587351 on the activation of p73 may, in part, share some signaling pathways involved in degradation of c-FLIP. The detailed molecular mechanism should be investigated more in the future.

c-FLIP is well-known to have high expression in various types of cancer, which is correlated with poor clinical outcomes. In our study, we report dynamic changes of c-FLIP expression as a transcription target gene of p73, which results in cell survival following p73-targeting therapy. The role of c-FLIP in p73-mediated determination of cell fate can be exploited more for therapeutic targeting of p73 in cancer therapy. Our study provides an attractive novel approach for targeting c-FLIP-L degradation by “switch compound” CB-7587351 to achieve anti-tumor efficacy through p73-targeting cancer therapy and exploitation of cell fate decisions downstream of p73 signaling leading to apoptosis.

### Example 2. Forming of Small Molecule Hydrates, Salts and Solvates

The small molecules disclosed herein can be provided as hydrates, solvates and/or as salts. For example, to form a salt any of the small molecules can be dissolved and brought to a charging pH along with a co-anion or a co-cation.

According to some aspects, a pharmaceutically acceptable salt described above or below includes an anion comprising bromide, carbide, chloride, fluoride, hydride, iodide, nitride, phosphide, oxide, sulfide, selenide, azide, peroxide, triodide, carbonate, chlorate, chromate, dichromate, dihydrogen phosphate, hydrogen carbonate, hydrogen sulfate, hydrogen sulfite, hydroxide, hypochlorite, monohydrogen phosphate, nitrate, nitrite, perchlorate, permanganate, peroxide, phosphate, sulfate, sulfite, superoxide, thiosulfate, silicate, metasilicate, aluminium silicate, acetate, formate, or oxalate.

For example, the chemical structure shown below can represent any of the small molecules disclosed herein with the Cl− being an anion disclosed herein:

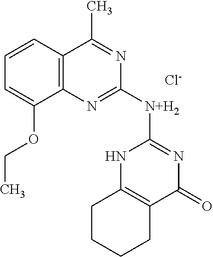

### Example of a Small Molecule Salt

In the example above, the anion shown as Cl− can be any pharmaceutically acceptable anion and can be associated at any charge point on the chemical structure (at various pH values), and there can be multiple anions in some bis-or tri-salt forms. The invention contemplates that any of the above-described compounds, methods, devices or combinations can be derivatized or can be structurally altered to further save lives, for example, by addition or substitution of one or more atoms using a radioisotope or using a different element (e.g., B or boron in place of C or carbon), by removal of an ester or by addition of a salt form, an amino acid, a sugar, or a peptide. Hydrates and/or solvates can be formed by 1) dissolving the molecule in water and/or solvent and slowly drying, whereby water and/or solvent remain hydrogen bonded with OH groups in the molecule or associated with the molecule. A formation of a hydrate or solvate can typically be confirmed by an attenuated total reflection (ATR) infrared spectrum acquired from the solid-state sample. The ATR spectrum of a hydrate or solvate will typically show increased broad bands (indicating hydrogen bonding) above about 3200 cm⁻¹, as compared to the non-hydrate or non-solvate solid sample. In some embodiments, the above-described compounds are attached to or associated with a targeting moiety. In some embodiments, the targeting moiety is a particle or an antibody with affinity for a specific type of cell. While various crystal structures are contemplated, these investigations will also be initiated.

### REFERENCES

1. Melino, G., V. De Laurenzi, and K. H. Vousden, p73: Friend or foe in tumorigenesis. Nat Rev Cancer, 2002. 2 (8): p. 605-15.
2. Dotsch, V., et al., p63 and p73, the ancestors of p53. Cold Spring Harb Perspect Biol, 2010. 2 (9): p. a004887.
3. Tomasini, R., et al., TAp73 knockout shows genomic instability with infertility and tumor suppressor functions. Genes Dev, 2008. 22 (19): p. 2677-91.
4. Conforti, F., et al., Regulation of p73 activity by post-translational modifications. Cell Death Dis, 2012. 3: p. e285.
5. Gong, J. G., et al., The tyrosine kinase c-Abl regulates p73 in apoptotic response to cisplatin-induced DNA damage. Nature, 1999. 399 (6738): p. 806-9.
6. Tsai, K. K. and Z. M. Yuan, c-Abl stabilizes p73 by a phosphorylation-augmented interaction.
Cancer Res, 2003. 63 (12): p. 3418-24.
7. Wade, M., Y. C. Li, and G. M. Wahl, MDM2, MDMX and p53 in oncogenesis and cancer therapy. Nat Rev Cancer, 2013. 13 (2): p. 83-96.
8. Zhang, S., et al., Advanced Strategies for Therapeutic Targeting of Wild-Type and Mutant p53 in Cancer. Biomolecules, 2022. 12 (4).
9. Wang, W., S. H. Kim, and W. S. El-Deiry, Small-molecule modulators of p53 family signaling and antitumor effects in p53-deficient human colon tumor xenograft.Proc Natl Acad Sci U S A, 2006. 103 (29): p. 11003-8.
10. Zhang, S., et al., Small-Molecule NSC59984 Restores p53 Pathway Signaling and Antitumor Effects against Colorectal Cancer via p73 Activation and Degradation of Mutant p53. Cancer Res, 2015. 75 (18): p. 3842-52.
11. Kravchenko, J. E., et al., Small-molecule RETRA suppresses mutant p53-bearing cancer cells through a p73-dependent salvage pathway. Proc Natl Acad Sci U S A, 2008. 105 (17): p. 6302-7.
12. Hong, B., et al., Prodigiosin rescues deficient p53 signaling and antitumor effects via upregulating p73 and disrupting its interaction with mutant p53. Cancer Res, 2014. 74 (4): p. 1153-65.
13. Nemajerova, A. and U. M. Moll, Tissue-specific roles of p73 in development and homeostasis. J Cell Sci, 2019. 132 (19).
14. Sharif, T., et al., TAp73 Modifies Metabolism and Positively Regulates Growth of Cancer Stem-Like Cells in a Redox-Sensitive Manner. Clin Cancer Res, 2019. 25 (6): p. 2001-2017.
15. Wang, C., C. R. Teo, and K. Sabapathy, p53-Related Transcription Targets of TAp73 in Cancer Cells-Bona Fide or Distorted Reality? Int J Mol Sci, 2020. 21 (4).
16. Carneiro, B. A. and W. S. El-Deiry, Targeting apoptosis in cancer therapy. Nat Rev Clin Oncol, 2020. 17 (7): p. 395-417.
17. Hernandez Borrero, L. J. and W. S. El-Deiry, Tumor suppressor p53: Biology, signaling pathways, and therapeutic targeting. Biochim Biophys Acta Rev Cancer, 2021. 1876 (1): p. 188556.
18. Hughes, M. A., et al., Co-operative and Hierarchical Binding of c-FLIP and Caspase-8: A Unified Model Defines How c-FLIP Isoforms Differentially Control Cell Fate. Mol Cell, 2016. 61 (6): p. 834-49.
19. Smyth, P., et al., FLIP(L): the pseudo-caspase. FEBS J, 2020. 287 (19): p. 4246-4260.
20. Borrelli, S., et al., p63 regulates the caspase-8-FLIP apoptotic pathway in epidermis. Cell Death Differ, 2009. 16 (2): p. 253-63.
21. Bartke, T., et al., p53 upregulates cFLIP, inhibits transcription of NF-kappaB-regulated genes and induces caspase-8-independent cell death in DLD-1 cells. Oncogene, 2001. 20 (5): p. 571-80.
22. Lees, A., et al., The pseudo-caspase FLIP(L) regulates cell fate following p53 activation. Proc Natl Acad Sci U S A, 2020. 117 (30): p. 17808-17819.
23. Gonzalez, R., et al., Role of p63 and p73 isoforms on the cell death in patients with hepatocellular carcinoma submitted to orthotopic liver transplantation. PLOS One, 2017. 12 (3): p. e0174326.
24. Zhang, S., L. Zhou, and W. S. El-Deiry, Small-Molecule NSC59984 Induces Mutant p53 Degradation through a ROS-ERK2-MDM2 Axis in Cancer Cells. Mol Cancer Res, 2022. 20 (4): p. 622-636.
25. Jackson, J. G. and O. M. Pereira-Smith, p53 is preferentially recruited to the promoters of growth arrest genes p21 and GADD45 during replicative senescence of normal human fibroblasts. Cancer Res, 2006. 66 (17): p. 8356-60.
26. Ricci, M. S., et al.,Direct repression of FLIP expression by c-myc is a major determinant of TRAIL sensitivity. Mol Cell Biol, 2004. 24 (19): p. 8541-55.
27. Cory, S. and J. M. Adams, The Bcl2 family: regulators of the cellular life-or-death switch. Nat Rev Cancer, 2002. 2 (9): p. 647-56.
28. Chang, L., et al., The E3 ubiquitin ligase itch couples JNK activation to TNFalpha-induced cell death by inducing c-FLIP(L) turnover. Cell, 2006. 124 (3): p. 601-13.
29. Wang, Q., et al., Down-regulation of cellular FLICE-inhibitory protein (Long Form) contributes to apoptosis induced by Hsp90 inhibition in human lung cancer cells. Cancer Cell Int, 2012. 12 (1): p. 54.
30. Dickens, L. S., et al., A death effector domain chain DISC model reveals a crucial role for caspase-8 chain assembly in mediating apoptotic cell death. Mol Cell, 2012. 47 (2): p. 291-305.
31. Rozenberg, J. M., et al., Dual Role of p73 in Cancer Microenvironment and DNA Damage Response. Cells, 2021. 10 (12).
32. Bennett, M., et al., Cell surface trafficking of Fas: a rapid mechanism of p53-mediated apoptosis. Science, 1998. 282 (5387): p. 290-3.
33. Sturmlechner, I., et al., p21 produces a bioactive secretome that places stressed cells under immunosurveillance. Science, 2021. 374 (6567): p. eabb3420.
34. Walczak, H. and P. H. Krammer, The CD95 (APO-1/Fas) and the TRAIL (APO-2L) apoptosis systems. Exp Cell Res, 2000. 256 (1): p. 58-66.
35. Krueger, A., et al., Cellular FLICE-inhibitory protein splice variants inhibit different steps of caspase-8 activation at the CD95 death-inducing signaling complex. J Biol Chem, 2001. 276 (23): p. 20633-40.
36. Humphreys, L. M., et al., A revised model of TRAIL-R2 DISC assembly explains how FLIP(L) can inhibit or promote apoptosis. EMBO Rep, 2020. 21 (3): p. e49254.
37. Sanchez-Rivera, F. J., et al., Mitochondrial apoptotic priming is a key determinant of cell fate upon p53 restoration. Proc Natl Acad Sci U S A, 2021. 118 (23).
38. Uramoto, H., et al., p73 Interacts with c-Myc to regulate Y-box-binding protein-1 expression. J Biol Chem, 2002. 277 (35): p. 31694-702.
39. Watanabe, K., et al., Physical interaction of p73 with c-Myc and MM1, a c-Myc-binding protein, and modulation of the p73 function. J Biol Chem, 2002. 277 (17): p. 15113-23.

We claim:

1. A method for treating cancer in a subject in need thereof, the method comprising the steps of:
(1) obtaining a subject with cancer, or a subject suspected of having cancer, or a subject who is susceptible to cancer;
(2) administering a therapeutically effective amount of a p73 protein activator to the subject, wherein the p73 protein activator is a switcher compound;
(3) wherein the administering of the switcher compound is configured to provide a reduction of a c-FLIP-L/S gene expression which is operative to sensitize cancer cells to a p73-induced apoptosis in the cancer cells;
thereby treating and/or preventing the cancer in the subject.

2. The method of claim 1, wherein the p73 activating switcher compound is administered in an amount effective to provide a reduction of c-FLIP-L/S gene expression operative to sensitize the cancer cells to p73-induced apoptosis.

3. The method of claim 1, wherein a related p53 tumor suppressor protein is inactive in the cancer cells.

4. The method of claim 1, where the administration of the switcher compound is configured to compensate for a loss of a protein p53's function by inducing cell cycle arrest, apoptosis or programmed cell death, and/or differentiation, thereby preventing an uncontrolled cancer cell growth and tumor development.

5. The method of claim 1, wherein the switcher compound activates p73 which transcriptionally regulates the c-FLIP gene and/or an expression of the c-FLIP gene.

6. The method of claim 1, wherein the switcher compound activates p73 as an orthosteric activator and/or an allosteric activator.

7. The method of claim 1, wherein the switcher compound is effective to restore wild-type function to a mutant p53 in one or more cancer cells in the subject.

8. The method of claim 1, further comprising the switcher compound is a modulator of the p73 either in addition to being an activator and/or instead of being an activator.

9. The method of claim 1, wherein the switcher compound comprises small molecule CB-7587351, small molecule RETRA, small molecule Prodigiosin, small molecule NSC59984, or a combination thereof; and/or a salt, hydrate and/or a solvate thereof.

10. The method of claim 1, wherein the switcher compound comprises one or more of: small molecule PRIMA-1 or PRIMA-1MET; small molecule MIRA-1; small molecule STIMA-1; small molecule 3-Benzoylacrylic acid; small molecule Prodigiosin; small molecule Nutlin-3; small molecule RITA; small molecule Stictic acid; small molecule CP-31398; small molecule RETRA; small molecule NSC59984; small molecule CB-7587351; and/or a salt, hydrate and/or a solvate thereof.

11. A composition comprising a p73 protein activator, wherein the p73 protein activator is a switcher compound selected from the group consisting of:
2-[(E)-2-(3,4-dihydroxyphenyl)ethenyl]-1-benzofuran-6-ol (CB-7587351);
2,5-bis(5-hydroxymethyl-2-thienyl)furan (RETRA);
4-methoxy-5-[(Z)-2-pyridin-3-ylvinyl]-1H-pyrrole-2-carbaldehyde (Prodigiosin); and/or
2-[(E)-2-(4-nitrophenyl)ethenyl]-1-benzofuran-5-ol (NSC59984);
and/or a salt, hydrate and/or a solvate thereof;
wherein the composition is an effective switcher compound in a method comprising the steps of:
(1) obtaining a subject with cancer, or a subject suspected of having cancer, or a subject who is susceptible to cancer; (2) administering a therapeutically effective amount of a p73 protein activator to the subject, wherein the p73 protein activator is a switcher compound; wherein the administering of the switcher compound is configured to provide a reduction of a c-FLIP-L/S gene expression which is operative to sensitize cancer cells to a p73-induced apoptosis in the cancer cells;
thereby treating and/or preventing the cancer in the subject.

12. The composition of claim 11, wherein the switcher compound is a modulator of the p73 either in addition to being an activator and/or instead of being an activator.

13. The composition of claim 11, wherein the switcher compound is effective to restore wild-type function to a mutant p53 in one or more cancer cells.

14. The composition of claim 11, wherein the switcher compound activates p73 as an orthosteric activator and/or an allosteric activator.

15. The composition of claim 11, wherein the switcher compound activates p73 which transcriptionally regulates the c-FLIP gene and/or an expression of the c-FLIP gene.

16. A method of identifying a population of humans wherein an activation and/or a modulation of a p73 protein by an administering of a small-molecule switcher compound or a less than 1000 molecular weight switcher compound will provide a reduction of a c-FLIP-L/S gene expression which is operative to sensitize any cancer cells in the human population to a p73-induced apoptosis in the cancer cells, the method comprising the steps of:
(1) obtaining a biological sample from each human in the population and/or obtaining data that has been previously derived from a biological sample from each human in the population;
(2) analyzing the biological samples and/or data to determine a level of p73 protein expression and/or activity in the cancer cells;
(3) identifying a subpopulation of humans having a low level of p73 protein expression and/or activity in the cancer cells compared to a control;
wherein the identified subpopulation of humans is the population wherein an activation and/or a modulation of the p73 protein by the administering of the switcher compound will provide the reduction of the c-FLIP-L/S gene expression which is operative to sensitize the cancer cells to the p73-induced apoptosis.

17. The method of claim 16, wherein the switcher compound comprises small molecule CB-7587351, small molecule RETRA, small molecule Prodigiosin, small molecule NSC59984, or a combination thereof; and/or a salt, hydrate and/or a solvate thereof.

18. The method of claim 16, wherein the switcher compound is a modulator of the p73 either in addition to being an activator and/or instead of being an activator.

19. The method of claim 16, wherein the switcher compound activates p73 as an orthosteric activator and/or an allosteric activator.

20. The method of claim 16, wherein the switcher compound activates p73 which transcriptionally regulates the c-FLIP gene and/or an expression of the c-FLIP gene.
